(19) United States
(12) Patent Application Publication
(10) Pub. No.: US 2014/0230091 A1
(43) Pub. Date: Aug. 14, 2014

(54) MODIFIED PLANTS WITH INCREASED OIL CONTENT

(71) Applicants: John Shanklin, Shoreham, NY (US); Carl Andre, Raleigh, NC (US)

(72) Inventors: John Shanklin, Shoreham, NY (US); Carl Andre, Raleigh, NC (US)

(73) Assignee: BROOKHAVEN SCIENCE ASSOCIATES, LLC, Upton, NY (US)

(21) Appl. No.: 14/130,039

(22) PCT Filed: Jun. 28, 2012
(86) PCT No.: PCT/US12/44676

§ 371 Date: Apr. 21, 2014

Related U.S. Application Data
(60) Provisional application No. 61/502,163, filed on Jun. 28, 2011.

(51) Int. Cl. C12N 15/82

(52) U.S. Cl. CPC C12N 15/8247
USPC 800/281; 800/298; 435/419; 536/23.2; 530/370; 585/240; 426/615; 426/635

(57) ABSTRACT

Methods and means are provided to increase the oil content of plants, particularly oleaginous plants by preventing feedback inhibition by 18:1-Coenzyme A or 18:1-Acyl Carrier Protein of the acetyl CoA-carboxylase enzyme in cells of these plants in various manners.

### CROSS REFERENCE TO RELATED APPLICATIONS AND INCORPORATION OF SEQUENCE LISTING

This application claims the benefit of priority of U.S. Provisional Patent Application 61/502,163 filed Jun. 28, 2011, which is incorporated by reference in its entirety herein. The sequence listing that is contained in the file named “58764000510PCT” which is 276 kb (measured in operating system MS-Windows) and was created on Jun. 15, 2012, is filed herewith and incorporated herein by reference.

This invention was made with Government support under contract number DE-AC02-98CH10886, awarded by the U.S. Department of Energy. The Government has certain rights in the invention.

### FIELD OF THE INVENTION

The invention relates to the field of agronomy. More particularly, the invention provides methods and means to increase the oil content of plants, particularly oleaginous plants by preventing feedback inhibition by 18:1-Coenzyme A or 18:1-Acyl Carrier Protein of the acetyl CoA-carboxylase enzyme in cells of these plants in various manners, including by providing feedback insensitive or less sensitive acetyl CoA-carboxylase enzymes, by overexpression of FATA genes or AcetylCoA binding proteins.

### BACKGROUND ART

Vegetable oils are increasingly important economically because they are widely used in human and animal diets and in many industrial applications, including as a renewable source to produce biofuel or biodiesel. The most widely used vegetable oils are derived from palm (world consumption 41.31 million tons in 2008) or soybean (41.28 million tons), followed by rapeseed oil (18.24), sunflower oil (9.91), peanut oil (4.82) cottonseed oil (4.99) palm kernel oil (4.85) coconut oil (3.48) and olive oil (2.84). Other significant triglyceride oils include corn oil, grape seed oil, hazelnut oil, linseed oil, rice bran oil, safflower oil and sesame oil.

Increasing the oil yield per plant seems to be a promising approach to provide more oil to be used for these different purposes, and avoiding land competition between oil for food and feed use on the one hand and for industrial use on the other hand. Oil synthesis in plants appears to be limited by the production of fatty acids, and the first committed step in fatty acid biosynthesis, i.e. the carboxylation of acetyl-CoA to produce malonyl-CoA by acetyl-CoA carboxylase, has been suggested to be rate-limiting.

Roesler et al. 1997 (Plant Physiol. 113, 75-81) described that expression of Arabidopsis homomeric acetyl-CoA carboxylase and targeting the protein to plastids of rapeseed resulted in a 5% increase in seed oil.

Madoka et al. 2002 (Plant Cell Physiol. 43, 1518-1525) reported that overexpression of the plastid-endoded Acetyl CoA carboxylase carboxytransferase beta-subunit (accD) by plastid transformation, induced an increase of 5-10% of leaf oil content in tobacco plants.

U.S. Pat. No. 5,962,767 describes the isolation of an Arabidopsis acetyl coA carboxylase gene encoding the 251 kD cytosolic acetyl CoA carboxylase.

WO94/17188 discloses another DNA sequence with codes for a plant acetyl-CoA carboxylase as well as alleles and derivatives of said DNA sequence.

WO95/13390 relates to plant thioesterases, specifically plant acyl-ACP thioesterases having substantial activity on palmitoyl-ACP substrates. DNA constructs useful for the expression of a plant palmitoyl-ACP thioesterase in a plant seed cell are described. Such constructs will contain a DNA sequence encoding the plant palmitoyl-ACP thioesterase of interest under the control of regulatory elements capable of preferentially directing the expression of the plant palmitoyl-ACP thioesterase in seed tissue, as compared with other plant tissues, when such a construct is expressed in a transgenic plant. The document also describes methods of using a DNA sequence encoding a plant palmitoyl-ACP thioesterase for the modification of the proportion of free fatty acids produced in a plant seed cell. Plant palmitoyl-ACP thioesterase sequences exemplified herein include Cuphea, leek, mango and elm. Transgenic plants having increased levels of C16:0 fatty acids in their seeds as the result of expression of these palmitoyl-ACP thioesterase sequences are also provided.

WO00/09721 relates to a method for increasing stearate as a component of total triglycerides found in soybean seed. The method generally comprises growing a soybean plant having integrated into its genome a DNA construct comprising, in the 5′ to 3′ direction of transcription, a promoter functional in a soybean plant seed cell, a DNA sequence encoding an acyl-ACP thioesterase protein having substantial activity on C18:0 acyl-ACP substrates, and a transcription termination region functional in a plant cell. The document also provides a soybean seed with about 33 weight percent or greater stearate as a component of total fatty acids found in seed triglycerides.

US2010/033329 describes methods using acyl-CoA binding proteins to enhance low-temperature tolerance in genetically modified plants.

US2009/0291479 describes manipulation of acyl-CoA binding proteins for altered lipid production in microbial hosts.

U.S. Pat. No. 7,880,053 describes method of using transformed plants expressing plant-derived acyl-coenzyme A-binding proteins in phytoremediation.

US2008/0229451 describes expression of microbial proteins in plants for production of plants with improved properties.

Feedback regulation of biosynthetic pathways optimizes cellular economy by communicating the demand for metabolites to the enzymes which supply them. Typically, feedback occurs when a downstream metabolite accumulates and causes inhibition of a rate limiting enzyme for its own production, thereby restricting flux through an entire pathway. Unfortunately such mechanisms, when unknown or poorly understood, can act as barriers to successful metabolic engineering. Plant fatty acid biosynthesis is one such pathway targeted for manipulation that displays feedback inhibition (Ramli et al. 2002, Biochem J, 364, 385-391, Shintani and Ohlrogge 1995, Plant J, 7, 577-587, Terzaghi 1986 Plant Physiol, 82, 780-786.). However, the mechanism and target(s) of feedback have not been determined. A more thorough understanding of this basic process will aid in the design and analysis of future engineering attempts at increasing fatty acid production in plants.

Animals, fungi, and bacteria have known mechanisms for feedback regulation of fatty acid biosynthesis. A common feature among these is inhibition of the enzyme acetyl-CoA carboxylase (ACCase, EC 6.4.1.2), which alone produces malonyl-CoA for fatty acid synthesis and is considered the rate limiting step of fatty acid synthesis (Cronan and Waldrop 2002 Prog Lipid Res, 41, 407-435, Ohlrogge and Jaworski 1997 Rev Plant Physiol Plant Mol Biol., 48, 109-136., Wakil et al. 1983 Annu Rev Biochem, 52, 537-579). In rats and yeast, palmitoyl-CoA, an end product of fatty acid synthesis, binds to and inhibits ACCase (Ogiwara et al. 1978 Eur J Biochem, 89, 33-41.). In addition, yeast ACCase and fatty acid synthase (FAS) gene expression are lowered by overnight exposure to long chain fatty acids in an acyl-CoA dependent manner (Feddersen et al. 2007 Biochem J, 407, 219-230). Bacteria have similar responses. The E. coli ACCase and beta-keto acyl-acyl carrier protein synthase (KAS) are both inhibited by long chain (C16-C18) acyl-acyl carrier protein (acyl-ACP), an intermediate of fatty acid synthesis (Davis and Cronan 2001 J Bacteriol, 183, 1499-1503, Heath and Rock 1995 J Bioi Chem, 270, 15531-15538). Growth in the presence of exogenous fatty acids also results in repression of bacterial fatty acid biosynthetic genes (including ACCase) by interaction of long chain acyl-ACP or acyl-CoA with transcription factors (Zhang and Rock 2009 J. Lipid Res, 50 Suppl, SI15-119). Based on these studies a picture has emerged in which lower demand for de novo fatty acids is signaled by the accumulation of acyl-ACP and/or acyl-CoA. These metabolites allosterically inhibit ACCase and can therefore rapidly restrict the production of malonyl-Co A for use in fatty acid synthesis. In conditions where the levels of acyl-ACP and acyl-CoA are not reduced following inhibition of ACCase, the expression of genes for the entire fatty acid biosynthetic pathway is repressed. When combined, these responses prevent the unnecessary production of fatty acids during periods of acute and chronic reduction in cellular demand.

A mechanism for feedback regulation of fatty acid synthesis in plants has not been determined. Tween-fatty acid esters are effective for feeding fatty acids (Terzaghi 1986 Plant Physiol, 82, 771-779.), and have been shown to cause feedback inhibition in tobacco (Shintani and Ohlrogge 1995, Plant J. 7, 577-587) and soybean (Terzaghi 1986 Plant Physiol, 82, 780-786) cell cultures and in oil palm and olive calli (Ramli et al. 2002 Biochem J, 364, 385-391). Based on the rate of synthesis of acyl-ACPs and ACCase protein levels in tobacco, Shintani and Ohlrogge hypothesized that feedback occurs through biochemical or post-translational modification of ACCase and possibly FAS. Purified maize and diatom ACCases were inhibited by palmitolyl-CoA (Nikolau and Hawke 1984 Arch Biochem Biophys, 228, 86-96, Roessler 1990 Planta, 198, 517-525), but long chain acyl-ACP failed to inhibit partially purified ACCases from castor and pea (Roesler et al. 1996 Planta, 198, 517-525). Medium chain acyl-ACPs did, however, inhibit KAS activity in crude extracts of canola and spinach (Bruck et al. 1996 Planta, 198, 271-278). The relevance of these results to feedback inhibition is unclear as changes in the steady state pools of acyl-CoA or acyl-ACP during feedback have not been measured. The situation is further complicated in plants due to the presence of structurally distinct ACCase and FAS systems in the plastid and in the cytosol, responsible for fatty acid synthesis and elongation, respectively. Whether or not the cytosolic elongation pathway is responsive to feedback is unknown. Previous studies used vegetative tissues or germinated seedlings to establish cell cultures, and so it is also not known if feedback occurs in tissues where high rates of fatty acid synthesis are required, such as oil seeds.

Thus, the prior art is deficient in teaching which isoform of Acetyl-CoA carboxylase is subject to feedback inhibition in plants, as well as which molecules are responsible for feedback inhibition. As described hereinafter, this problem has been solved, allowing to prevent or circumvent feedback inhibition of acetyl-CoA carboxylase in plant cells, plant parts, plant tissues, seeds and plants with the aim to increase fatty acid synthesis and oil synthesis, as will become apparent from the different embodiments and the claims.

### SUMMARY OF THE INVENTION

In one embodiment, the invention relates to a method to increase oil content in cells of a plant, comprising the step of preventing feedback inhibition by 18:1-Coenzyme A or 18:1-Acyl Carrier Protein of the plastidic acetyl CoA-carboxylase enzyme in the cells of the plant. This prevention of feedback inhibition can be achieved by providing the plant cell (including providing the plastids of the plant cell) with an acetyl CoA-carboxylase variant enzyme or subunit thereof which is less sensitive to the feedback inhibition than a wild-type acetyl CoA-carboxylase of the plant. The less sensitive acetyl CoA-carboxylase variant enzyme or subunit thereof may be encoded by a variant allele in the plant cell or may be encoded by transgene introduced into the plant cell.

In another embodiment of the invention, a method is provided to increase oil content in cells of a plant, comprising the step of preventing feedback inhibition by 18:1-Coenzyme A or 18:1-Acyl Carrier Protein of the plastidic acetyl CoA-carboxylase enzyme in the cells of the plant, wherein the plant cell is provided with an acetyl CoA-carboxylase enzyme or one or more subunits thereof, from an organism that uses the 3-hydroxypropionate cycle for carbon fixation, such as an acetyl CoA-carboxylase or subunit thereof from an organism selected from the group of Sulfolobales, Cenarchaeles, Archeaoglobales, Desulfurococcales, Thermoproteales, Thermococcales or Halobacterales. The acetyl CoA-carboxylase or subunit thereof may be derived from an organism selected from the group of Metallosphaera sedula, Acidianus brierleyi, Sulfolobus solfataricus, Sulfolobus tokodaii, Sulfolobus acidocaldaricus, Cenarcheum symbiosum, Archaeoglobus fulgidus, Hyperthennus butylicus, Staphylotthermus marinus, Thermofilum pendens, Ingicoccus hospitalis, Pyrobaculum aerophilum, Pyrobaculum islandicum, Pyrobaculum calidifontis, Pyrobaculum furisous, Pyrobaculum abyssi, Pyrobaculum hoiykoshii, Haloarcula marismortui, Halobacterium sp. NRC-1, Haloquatratum walsbyi, Halorubrum lacusprofundi or Natromonas pharaonis. The plant cell may also be provided with an acetyl CoA-carboxylase enzyme or subunit thereof from Chloroflexus auranticus.

In yet another embodiment of the invention, a method is provided to increase oil content in cells of a plant, comprising the step of providing the plant cell with a DNA molecule comprising the following operably linked DNA fragments:
a) a plant expressible promoter;
b) one or more coding regions encoding one or more acetyl CoA-carboxylase subunits having an amino acid sequence selected from the amino acid sequences of SEQ ID Nos. 2, 3, 5, 7, 9, 11, 13, 15, 17 or 19, preferably a heterologous coding region; or a coding region having 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with an amino acid sequence selected from the amino acid sequence of SEQ ID Nos. 2, 3, 5, 7, 9, 11, 13, 15, 17 or 19, and having acetylCoA carboxylase enzymatic activity; and optionally
c) a transcription termination and/or polyadenylation region functional in plant cells.
   The DNA molecule may further comprises a DNA region encoding a chloroplast targeting peptide. The coding region may be selected from the nucleotide sequence of SEQ ID No. 1 from the nucleotide at position 331 to the nucleotide at position 1860, SEQ ID No. 1 from the nucleotide at position 1860 to the nucleotide at position2360, SEQ ID No. 4, SEQ ID No 6, SEQ ID No 8, SEQ ID No 10, SEQ ID No 12, SEQ ID No 14, SEQ ID No 16 or SEQ ID No 18 or a coding region having 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% nucleotide sequence identity therewith. In a particular embodiment, the plant expressible promoter is a promoter which is expressed in plastids and wherein the termination and/or polyadenylation region is a transcription termination region functional in plastids. The DNA molecule may be integrated in the nuclear genome of the plant cell or alternatively, the DNA molecule may be integrated in the genome of plastids of the plant cell. The plant cell may also contain more than one DNA molecule each expressing one subunit of an acetyl-CoA carboxylase enzyme.

The invention also provides a method, a method is provided to increase oil content in cells of a plant, comprising the step of preventing feedback inhibition by 18:1-Coenzyme A or 18:1-Acyl Carrier Protein of the plastidic acetyl CoA-carboxylase enzyme in the cells of the plant wherein the prevention of feedback inhibition is achieved by reducing the level of 18:1-Coenzyme A or 18:1-Acyl Carrier Protein in the plastids of the plant cell. One alternative embodiment to reduce the level of 18:1-Coenzyme A or 18:1-Acyl Carrier Protein in the plastids is by increasing the level of FATA enzyme in the plastids of the cell. To this end, a DNA molecule comprising a plant expressible promoter, operably linked to a DNA region encoding a FATA enzyme, such as the FATA enzyme having an amino acid sequence selected from the amino acid sequence of SEQ ID 21 or an amino acid sequence having 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity therewith; and optionally a transcription termination and/or polyadenylation region functional in plant cells may be introduced into the plant cell. Again, the DNA molecule may further comprise a DNA region encoding a chloroplast targeting peptide, or the plant expressible promoter is a promoter which is expressed in plastids and wherein the termination and/or polyadenylation region is a transcription termination region

In yet another embodiment of the invention, a method for increasing oil content in cells of a plant is provided, comprising the step of preventing feedback inhibition by 18:1-Coenzyme A or 18:1-Acyl Carrier Protein of the plastidic acetyl CoA-carboxylase enzyme in the cells of the plant, wherein the reduction of the level of 18:1-Coenzyme A or 18:1-Acyl Carrier Protein in the plastids is achieved by increasing the level of Acyl-CoA binding proteins in the plant cell. To this end, a DNA molecule may be introduced into the plant cell, wherein the DNA molecule comprises a plant expressible promoter operably linked to a DNA region encoding an Acyl-CoA binding protein; and optionally a transcription termination and/or polyadenylation region functional in plant cells. The Acyl-CoA binding protein may comprise an amino acid sequence selected from the amino acid sequence of any of SEQ ID No 23 or SEQ ID No 25 or an amino acid sequence having 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity therewith.

The plant cells in any of the mentioned methods may be regenerated into a plant. Accordingly, the invention also provides methods as described hereinbefore, wherein the plant cell is in a plant; and wherein the oil content is increased in oil storage parts, such as seeds, of the plant.

The methods may be applied to any plant, but are particularly useful in oleipherous plant such as Brassica oilseeds, including Brassica napus, Brassica campestris (rapa), Brassica juncea or Brassica carinata, sunflower, safflower, soybean, palm, Jatropha, flax, crambe, camelina, corn, sesame, castor beans.

The invention further provides a plant comprising one or more plastidic ACCase variant enzymes or subunits thereof which are less sensititve to feedback inhibition by 18:1-Coenzyme A or 18:1-Acyl Carrier Protein than a wild-type acetyl CoA-carboxylase of the plant such as a CoA-carboxylase enzyme or subunit thereof from an organism that uses the 3-hydroxypropionate cycle for carbon fixation, particularly wherein the acetyl CoA-carboxylase or subunit thereof is from an organism selected from the group of Sulfolobales, Cenarchaeles, Archeaoglobales, Desulfurococcales, Thermoproteales, Thermococcales or Halobacterales such as Metallosphaera sedula, Acidianus brierleyi, Sulfolobus solfataricus, Sulfolobus tokodaii, Sulfolobus acidocaldaricus, Cenarcheum symbiosum, Archaeoglobus fulgidus, Hyperthermus butylicus, Staphylotthermus marinus, Thermofilum pendens, Ingicoccus hospitalis, Pyrobaculum aerophilum, Pyrobaculum islandicum, Pyrobaculum calidifontis, Pyrobaculum furisous, Pyrobaculum abyssi, Pyrobaculum horykoshii, Haloarcula marismortui, Halobacterium sp. NRC-1, Haloquatratum walsbyi, Halorubrum lacusprofundi or Natromonas pharaonis. The acetyl CoA-carboxylase enzyme or subunit thereof may also be from Chloroflexus auranticus.

In yet an alternative embodiment, the invention provides a plant comprising a DNA molecule comprising the following operably linked DNA fragments:
a. a plant expressible promoter;
b. one or more coding regions encoding one or more acetyl CoA-carboxylase subunits having an amino acid sequence selected from the amino acid sequence of SEQ ID Nos. 2, 3, 5, 7, 9, 11, 13, 15, 17 or 19, preferably a heterologous coding region; or a coding region having 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with an amino acid sequence selected from the amino acid sequence of SEQ ID Nos. 2, 3, 5, 7, 9, 11, 13, 15, 17 or 19., and having acetylCoA carboxylase enzymatic activity, such as a coding region comprising the nucleotide sequence of SEQ ID No. 1 from the nucleotide at position 331 to the nucleotide at position 1860, SEQ ID No. 1 from the nucleotide at position 1860 to the nucleotide at position 2360, SEQ ID No. 4, SEQ ID No 6, SEQ ID No 8, SEQ ID No 10, SEQ ID No 12, SEQ ID No 14, SEQ ID No 16 or SEQ ID No 18 or a coding region having 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% nucleotide sequence identity therewith; and optionally
c. a transcription termination and/or polyadenylation region functional in plant cells.
   The DNA molecule may further comprise a DNA region encoding a chloroplast targeting peptide or the plant expressible promoter may a promoter which is expressed in plastids and the termination and/or polyadenylation region may be a transcription termination region functional in plastids. The plant cell may also contain more than one DNA molecule each expressing one subunit of an acetyl-CoA carboxylase enzyme.

The invention further provides cells, tissues, oil storage tissue or seeds of a plant as herein described, as well as oil produced from such a plant.

It is yet another object of the invention to provide a chimeric DNA comprising the following operably linked DNA fragments
a. a plant expressible promoter;
b. one or more coding regions encoding one or more acetyl CoA-carboxylase subunits having an amino acid sequence selected from the amino acid sequence of SEQ ID Nos. 2, 3, 5, 7, 9, 11, 13, 15, 17 or 19, preferably a heterologous coding region; or a coding region having 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with an amino acid sequence selected from the amino acid sequence of SEQ ID SEQ ID Nos. 2, 3, 5, 7, 9, 11, 13, 15, 17 or 19 and having acetylCoA carboxylase enzymatic activity; and optionally
c. a transcription termination and/or polyadenylation region functional in plant cells.

The invention thus relates to the use of an acetyl CoA-carboxylase variant enzyme or subunit thereof which is less sensitive to feedback inhibition by 18:1-Coenzyme A or 18:1-Acyl Carrier Protein than a wild-type acetyl CoA-carboxylase in the plastids of a cell of a plant to increase the oil content in cells of a plant.

In yet another embodiment of the invention, a method is provided to isolate a variant of a plastidic acetyl CoA-carboxylase enzyme or subunit thereof which is less sensitive to feedback inhibition by 18:1-Coenzyme A or 18:1-Acyl Carrier Protein than a wild-type acetyl CoA-carboxylase of a plant comprising the step of
a. generating a multitude of variant acetyl CoA-carboxylase enzymes or subunits thereof derived from a feedback inhibition sensitive CoA-carboxylase preferably from a plant, particularly from the plastids of a plant;
b. identifying the enzymatic activity of each of the variant acetyl CoA-carboxylase enzymes or subunit thereof in the presence of 18:1-Coenzyme A or 18:1-Acyl Carrier Protein or 18:1 Tween;
c. isolating those enzyme variants or subunits thereof which have a greater enzymatic activity in the presence of 18:1-Coenzyme A or 18:1-Acyl Carrier Protein or 18:1 Tween than the enzymatic activity of the feedback inhibition sensitive CoA-carboxylase.

The invention also provides a method to increase oil content in cells of a plant comprising the steps of isolating a variant of acetyl CoA-carboxylase enzyme or subunit thereof which is less sensitive to feedback inhibition by 18:1-Coenzyme A or 18:1-Acyl Carrier Protein than a wild-type acetyl CoA-carboxylase of a plant; and introducing the variant of acetyl CoA-carboxylase enzyme or subunit thereof in a cell of plant, preferably by transcription from a DNA construct encoding the acetyl CoA-carboxylase or subunit thereof.

Still another object of the invention is a method to isolate a plant cell or plant comprising a variant allele encoding an acetyl CoA-carboxylase variant enzyme, such as a plastidic acetyl CoA-carboxylase variant enzyme, or subunit thereof which is less sensitive to feedback inhibition by 18:1-Coenzyme A or 18:1-Acyl Carrier Protein than a wild-type acetyl CoA-carboxylase of a plant comprising the steps of:
a. providing a population of plant cells or plants comprising a multitude of variant acetyl CoA-carboxylase enzymes or subunits thereof;
b. identifying the enzymatic activity of each of the variant acetyl CoA-carboxylase enzymes or subunits thereof in the presence of 18:1-Coenzyme A or 18:1-Acyl Carrier Protein or 18:1 Tween;
c. isolating those plant cells or plants comprising enzyme variants or subunits thereof which have a greater enzymatic activity in the presence of 18:1-Coenzyme A or 18:1-Acyl Carrier Protein than the enzymatic activity of the feedback inhibition sensitive CoA-carboxylase,

as well as plant cells or plant obtained by this method.

The invention further relates to a method of producing food, feed, or an industrial product comprising the steps of obtaining a plant as herein described and preparing the food, feed or industrial product from the plant or part thereof. The food or feed may be oil, meal, grain, starch, flour or protein; or the industrial product may be biofuel, fiber, industrial chemicals, a pharmaceutical or a nutraceutical.

### BRIEF DESCRIPTION OF THE DRAWINGS

FIG. 1—(a) Growth, (b) protein composition, and (c) lipid profile of cells grown of

various concentrations of Tween-80.

FIG. 2—Fatty acid content in B. napus cells after 8 clays of growth with various concentrations of Tween-80. (a) Fatty acids in polar lipids. (b) Fatty acids in triacylglycerol (TAG). (c) Total fatty acid content. All data are the mean±SD (n=3). FW, fresh weight.

FIG. 3—Quantification of fatty acid uptake from ¹³C-oleoyl-Tween by B. napus cells. Time course of appearance of 13C-fatty acids into (a) polar lipids and (b) triacylglycerol (TAG) when cells were fed 10 mM 13C-oleoyl-Tween. Dark areas are unlabeled endogenous fatty acids and light areas are 13C-fatty acids from Tween. Individual fatty acid species are listed in the top left corner of each graph. All data are the mean±SD (n=3). FW, fresh weight.

FIG. 4—Inhibition of 14C-acetate labeling of lipids by B. napus cells in the presence of Tween-80. (a) Time course showing incorporation of 14C-acetate into total lipids in the presence of 10 mM Tween-80. (b) ¹⁴C-actetate labeling of total lipids after 3 hours of exposure to various concentrations of Tween-80. (c) Time course of ¹⁴C-actetate labeling of total lipids after removal of Tween-80 following a 3 hour exposure to 10 mM Tween-0.80. All data are the mean±SD (n=3).

FIG. 5—¹⁴C-acetate incorporation into (a) sterols and (b) free fatty acids in the presence or absence of Tween-80.

FIG. 6—Specific inhibition of plastidic ACCase in B. napus cells after 3 hours of Tween-80 feeding. (a) Relative ¹⁴C-acetate incorporation into individual fatty acids after 3 hours of 10 mM Tween-80 feeding. (b) ¹⁴C-acetate incorporation into total lipids by cells fed various concentrations of haloxyfop after 3 hours of 10 mM Tween-80 feeding. (c) Incorporation of label from ¹⁴C-malonate and, ¹⁴C-acetate into 16 and 18 carbon fatty acids after 3 hours of 10 mM Tween-80 feeding. All data are the mean±SD (n=3).

FIG. 7—Effect of (a) haloxyfop, (b) pliosphatase treatment and 2-oxoglutarate, and (c) Tween-80 on ACCase enzyme activity from crude cell extracts.

FIG. 8—Effect of malonate on fatty acid content in (a) polar lipids and (b) TAG and on (c) ¹⁴C-acetate labeling of lipids.

FIG. 9—Quantification of lipid intermediates in B. napus cells after 3 hours of Tween-80 feeding. (a) Free fatty acid (FFA), (b) acyl-ACP, and (c) acyl-CoA content in cell after 3 hours of 10 mM Tween-80 feeding. Where present, numbers represent the percent of 18:1 that was ¹³C-labeled after 3 hours of 10 mM ¹³C-oleoyl-Tween feeding. All data are the mean±SD (n=3).

FIG. 10—Effects of lipid intermediates on ACCase activity in crude B. napus cell extracts. (a) Effect of 10 μM free fatty acid on ACCase activity in crude extracts. (b) Effect of B. napus 16:0- and 18:1-ACP on ACCase activity in crude extracts. (c) Effect of various long chain acyl-CoA on ACCase activity in crude extracts. All data are the mean SD (n=3).

FIG. 11—Model for proposed mechanism of feedback inhibition of fatty acid synthesis. Plastidic ACCase is inhibited by 18:1-ACP and 18:1-CoA. These metabolites are products of de novo fatty acid synthesis inside the plastid, or are synthesized from exogenous fatty acids provided by Tween-18:1. Reactions that can produce or consume, and therefore participate in the regulation of, 18:1-ACP or 18:1-CoA are indicated with arrows.

FIG. 12—Seed Oil content of Arabidopsis thaliana lines overexpressing the ACCase subunits of Cenarchaeum symbiosum (ACCase Line) compared to the seed oil content of Arabidopsis thaliana lines which have been transformed with the backbone T-DNA vector without the ACCase subunits (EVL: empty vector line). Fatty acid methyl ester (FAME) concentration was determined based on the analysis of 3 seed samples per line. The seeds analyzed were T2-seeds.

### DESCRIPTION OF DIFFERENT EMBODIMENTS OF THE INVENTION

The current invention is based on the identification of the target and the molecules effecting feedback inhibition of the initial step in the fatty acid biosynthesis. As demonstrated below, particularly in the examples, the inventors have identified that, in plants, it is specifically the plastidic, heteromeric form of acetyl-CoA carboxylase which is subject to feedback inhibition, and that the effector molecules are specifically oleolyl-ACP and oleolyl-CoA.

Accordingly, the invention provides a method for increasing the oil content in cells of a plant comprising the step of preventing feedback inhibition by 18:1-Coenzyme A or 18:1-Acyl Carrier Protein of the plastidic acetyl CoA-carboxylase enzyme in said cells of said plant.

In a first embodiment of the invention, the feedback inhibition is prevented by providing the plant cell, particularly the plastids of the plant cells with acetyl CoA carboxylase variant enzymes, or subunits thereof, which is less sensitive to said feedback inhibition than the corresponding wild-type acetyl CoA-carboxylase of the plant.

As used herein “Acetyl-CoA carboxylase” (ACC), E.C. number 6.4.1.2 is a biotin-dependent enzyme that catalyzes the first committed enzymatic step in fatty acid biosynthesis i.e. the irreversible carboxylation of acetyl-CoA to produce malonyl-CoA through its two catalytic activities, biotin carboxylase (BC) and carboxyltransferase (CT). The initial partial reaction is catalyzed by biotin carboxylase and uses bicarbonate and ATP to carboxylase via a carboxyphosphate intermediate the biotin prosthetic group attached to biotin carboxyl carrier protein (BCCP) via a lysine residue.

HCO₃⁻+ATP+BCCP=>ADP+Pi+BCCP-COO⁻

The carboxygroup is then transferred to acceptor acetyl-Coenzyme A to produce malonyl-Coenyme A, a reaction catalyzed by the carboxyltransferase.

BCCP-COO⁻+Acetyl-CoA=>Malonyl-CoA+BCCP

ACCs have been found in most living organisms, including archea, bacteria, yeast, fungi, plants, animals and humans. In most eukaryotes, ACC is a multi-domain enzyme (a homomeric form) whereby the BC, BCCP and CT activities are located on a large polypeptide (>200 kDa). Prokaryotes have multi-subunit ACCs composed of several polypeptides encoded by distinct genes. Biotin carboxylase (BC) activity, biotin carboxyl carrier protein (BCCP) is each contained on a different subunit, with the encoding genes usually referred to as accC and accB respectively. The carboxyl transferase (CT) activity is split over two peptides, α-carboxyl transferase (encoded by accA) and β-carboxyl transferase (encoded by accD). In Archea, the alpha and beta subunit are encoded by one gene. Most plants, except Graminea, contain both the heteromeric, “prokaryotic”, form and the homomeric “eukaryotic” form. The heteromeric form is located in the plastids and is used for the de novo synthesis of fatty acids. Three of the encoding genes (for biotin carboxylase, biotin carboxyl carrier protein, and the α-carboxyl transferase subunit) are nuclear encoded, while the gene coding for the β-carboxyl transferase is located on the plastid genome. The homomeric form is located outside of the plastids, in the cytosol, Graminea do not contain the “prokaryotic” form of ACC, but contain the homomeric form both in plastids and cytosol.

Assays for measuring ACC activity are well known in the art and include e.g. the assay utilizing measurement of phosphate to estimate enzymatic activity as described by Howard and Ridley, 1990 (FEES Letters 261, 2, 261-264 February 1990) or the spectrophotometric assay described by Kroeger et al., 2011 (Analytical BioChemistry 411, 100-105).

Numerous genes encoding ACC multidomain proteins or ACC subunits from plants have been isolated and protein sequences for ACC multidomain proteins or ACC subunits can be found in databases. The amino acid sequence of Arabidopsis thaliana homomeric ACC proteins can be found e.g. under Accession numbers NP_{—}174850 (acetyl-CoA carboxylase2) or NP_{—}174849 (acetyl-CoA carboxylase2). NP_{—}197143 (biotin carboxyl carrier protein of ACC 1), NP_{—}001031968 (biotin carboxylase) NP_{—}850291 (carboxyl transferase subunit alpha) and ACCD_ARATH (carboxyl transferase subunit beta) represent the amino acid sequences of the different subunits of the Arabidopsis thaliana heteromeric ACC.

The Accession numbers for the amino acid sequence of homomeric ACC proteins or of the different subunits of heteromeric proteins for Brassica napus, Brassica oleracea, Brassica rapa and Brassica juncea can be found in the following tables 1 to 5. All amino acid sequences are hereby incorporated by reference.

TABLE 1
Homomeric Acetyl CoA carboxylases from Brassica spp.

| Brassica | length | Brassica | length | Brassica | length | Brassica | length |
|---|---|---|---|---|---|---|---|
| napus | (AA) | oleracea | (AA) | rapa | (AA) | juncea | (AA) |
| CBU86998 | 2321 | | | ABA1005 | 1065 | | |
| CBV09123 | 2304 | | | ABA1006 | 1063 | | |
| CBV09287 | 2321 | | | ABA1007 | 1065 | | |
| CAA54683 | 2304 | | | | | | |
| CAC19875 | 2321 | | | | | | |
| CAA02452 | 2273 | | | | | | |
| CBU86834 | 2304 | | | | | | |
| CBU93391 | 2321 | | | | | | |
| CBU93227 | 2304 | | | | | | |
| CBU89179 | 2321 | | | | | | |
| CBU89015 | 2304 | | | | | | |
| CBU86094 | 2321 | | | | | | |
| CBU85930 | 2304 | | | | | | |
| CBU85485 | 2321 | | | | | | |
| CBU85321 | 2304 | | | | | | |
| CBF95767 | 2321 | | | | | | |
| CBF95603 | 2304 | | | | | | |
| CBF68517 | 2321 | | | | | | |
| CBF68353 | 2304 | | | | | | |
| CBF62122 | 2321 | | | | | | |
| CBF61958 | 2304 | | | | | | |
| CBF61066 | 2321 | | | | | | |
| CBF60793 | 2304 | | | | | | |
| CBF60241 | 2321 | | | | | | |
| CBF60077 | 2304 | | | | | | |
| CAC19876 | 1789 | | | | | | |
| ABA01004 | 1064 | | | | | | |
| CAC16410 | 796 | | | | | | |

TABLE 2
Heteromeric Acetyl CoA carboxylases from
Brassica spp- Biotin carboxylase subunit

| Brassica | length | Brassica | length | Brassica | length | Brassica | length |
|---|---|---|---|---|---|---|---|
| napus | (AA) | oleracea | (AA) | rapa | (AA) | juncea | (AA) |
| AAK60339 | 535 | ADI79330 | 536 | ADI79336 | 536 | ADI79330 | 536 |
| CAA71346 | 640 | ADI79331 | 535 | ADI79337 | 535 | ADI79331 | 535 |
| ADI79335 | 536 | | | | | | |
| ADI79334 | 536 | | | | | | |
| ADI79333 | 535 | | | | | | |
| ADI79332 | 535 | | | | | | |
| CBV09275 | 535 | | | | | | |
| CBU86986 | 535 | | | | | | |
| CBU93379 | 535 | | | | | | |
| CBU89167 | 535 | | | | | | |
| CBU86082 | 535 | | | | | | |
| CBU85473 | 535 | | | | | | |
| CBF95755 | 535 | | | | | | |
| CBF68505 | 535 | | | | | | |
| CBF62110 | 535 | | | | | | |
| CBF61054 | 535 | | | | | | |
| CBF60229 | 535 | | | | | | |
| CBF64291 | 535 | | | | | | |
| CAA71347 | 371 | | | | | | |

TABLE 3
Heteromeric Acetyl CoA carboxylases from Brassica
spp- Biotin carboxylase carrier protein

| Brassica | length | Brassica | length | Brassica | length | Brassica | length |
|---|---|---|---|---|---|---|---|
| napus | (AA) | oleracea | (AA) | rapa | (AA) | juncea | (AA) |
| CAA62265 | 251 | | | | | | |
| CAA62264 | 256 | | | | | | |
| AAS46758 | 260 | | | | | | |
| 2210244E | 251 | | | | | | |
| 2210244D | 256 | | | | | | |
| 2210244A | 192 | | | | | | |
| CAA62261 | 192 | | | | | | |
| CAA62263 | 162 | | | | | | |

TABLE 4
Heteromeric Acetyl CoA carboxylases from Brassica
spp- carboxyltransferase alpha subunit

| Brassica | length | Brassica | length | Brassica | length | Brassica | length |
|---|---|---|---|---|---|---|---|
| napus | (AA) | oleracea | (AA) | rapa | (AA) | juncea | (AA) |
| ACN65504 | 769 | ACN65502 | 770 | ACN65499 | 770 | | |
| ACN65503 | 764 | ACN65501 | 771 | ACN65500 | 768 | | |
| ACT83681 | 769 | | | | | | |
| ACT83680 | 765 | | | | | | |
| AAS46759 | 764 | | | | | | |

TABLE 5
Heteromeric Acetyl CoA carboxylases from Brassica spp- carboxyltransferase beta subunit

| Brassica | length | Brassica | length | Brassica | length | Brassica | length |
|---|---|---|---|---|---|---|---|
| napus | (AA) | oleracea | (AA) | rapa | (AA) | juncea | (AA) |
| ACY66222 | 489 | ABZ10596 | 489 | ABZ10594 | 487 | ABZ1095 | 487 |
| ACCD_BRANA | 489 | | | | | | |
| 2210244G | 489 | | | | | | |

One way to obtain acety-coA carboxylase variant enzyme or variant subunits thereof which are less sensitive to feedback inhibition by 18:1-ACP or 18:1-CoA is to isolate such variants starting from the amino acid sequences encoding biotin carboxylase, biotin carboxylase carrier protein and/or carboxyl transferase subunits, such as those mentioned or incorporated by reference herein, or their encoding nucleotide sequences, from plants.

To this end, a multitude of variant acetyl CoA-carboxylase enzymes or subunits thereof derived from a feedback inhibition sensitive CoA-carboxylase enzymes or subunits thereof, preferably from a plant, can be generated using methods conventional in the art of protein engineering. E.g. nucleotide sequences encoding ACCase or the subunits thereof may be subjected to PCR under error-prone conditions to create variants thereof. The variation may then even be enhanced using PCR to reassemble and shuffle these similar but not identical DNA sequences. Variant ACCase or their subunits may be expressed in host cells, such as E. coli or Saccharomyces cerevisae, Pichia pastoris, plant cells etc. Next, the enzymatic activity of these variant acetyl CoA-carboxylase enzymes or their subunits is identified, in the absence and presence of 18:1-Coenzyme A or 18:1-Acyl Carrier Protein or 18:1 Tween, as described herein, and those enzyme variants (or their subunits) which have a greater enzymatic activity in the presence of 18:1-Coenzyme A or 18:1-Acyl Carrier Protein than the enzymatic activity of said feedback inhibition sensitive CoA-carboxylase are isolated an optionally used to be introduced into the plastids of a plant cell.

Variant plastidic acetyl CoA-carboxylase enzymes or subunits thereof may also be generated in plant cells, by variant alleles. To this end, a population of plant cells or plants comprising a multitude of variant acetyl CoA-carboxylase enzymes or subunits thereof can be generated, e.g. through the use of mutagenesis. Again, the enzymatic activity of each of variant acetyl CoA-carboxylase enzymes or subunits thereof in the presence of 18:1-Coenzyme A or 18:1-Acyl Carrier Protein or 18:1 Tween is determined as herein described and those plant cells or plants comprising enzyme variants which have a greater enzymatic activity in the presence of 18:1-Coenzyme A or 18:1-Acyl Carrier Protein than the enzymatic activity of the feedback inhibition sensitive CoA-carboxylase are identified. Plant cells may be used to regenerate plants comprising the variant alleles. These plants may be used in further crosses to combine the required variant alleles in the plant varieties of choice.

“Mutagenesis”, as used herein, refers to the process in which plant cells (e.g., a plurality of plants seeds or other parts, such as pollen, etc.) are subjected to a technique which induces mutations in the DNA of the cells, such as contact with a mutagenic agent, such as a chemical substance (such as ethylmethylsulfonate (EMS), ethylnitrosourea (ENU), etc.) or ionizing radiation (neutrons (such as in fast neutron mutagenesis, etc.), alpha rays, gamma rays (such as that supplied by a Cobalt 60 source), X-rays, UV-radiation, etc.), or a combination of two or more of these. Thus, the desired mutagenesis of one or more ACCase encoding alleles may be accomplished by use of chemical means such as by contact of one or more plant tissues with ethylmethylsulfonate (EMS), ethylnitrosourea, etc., by the use of physical means such as x-ray, etc, or by gamma radiation, such as that supplied by a Cobalt 60 source. While mutations created by irradiation are often large deletions or other gross lesions such as translocations or complex rearrangements, mutations created by chemical mutagens are often more discrete lesions such as point mutations. For example, EMS alkylates guanine bases, which results in base mispairing: an alkylated guanine will pair with a thymine base, resulting primarily in G/C to A/T transitions. Following mutagenesis, plants can be regenerated from the treated cells using known techniques. For instance, the resulting seeds may be planted in accordance with conventional growing procedures and following self-pollination seed is formed on the plants. Alternatively, doubled haploid plantlets may be extracted to immediately form homozygous plants, for example as described by Coventry et al. (1988, Manual for Microspore Culture Technique for Brassica napus. Dep. Crop Sci. Techn. Bull. OAC Publication 0489. Univ. of Guelph, Guelph, Ontario, Canada). Additional seed that is formed as a result of such self-pollination in the present or a subsequent generation may be harvested and screened for the presence of mutant alleles. Several techniques are known to screen for specific mutant alleles, e.g., Deleteagene™ (Delete-a-gene; Li et al., 2001, Plant J 27: 235-242) uses polymerase chain reaction (PCR) assays to screen for deletion mutants generated by fast neutron mutagenesis, TILLING (targeted induced local lesions in genomes; McCallum et al., 2000, Nat Biotechnol 18:455-457) identifies EMS-induced point mutations, etc.

Another way to reduce feedback inhibition by 18:1-ACP or 18:1-CoA is to use feedback insensitive ACCases or subunits thereof isolated from other organisms, such as bacteria or archea which possess multisubunit ACCases that are involved in carbon fixation, but not in fatty acid synthesis. Included are organisms that use the so-called 3-hydroxypropionate cycle for carbon fixation (Hugler et al. 2003, Eur. J. Biochem. 270, 736-734). Characterization of one of these ACCases indicated that indeed it is not inhibited by acyl-CoAs (Chuakrut et al. 2003, J. Bacterial. 1.85(3):938-947).

Thus, a method is provided to increase oil content in cells of a plant by providing the plastids of cells of the plant with an acetyl CoA-carboxylase or subunit thereof from Sulfolobales, Cenarchaeles, Archeaoglobales, Desulfurococcales, Thermoproteales, Thermococcales or Halobacterales such as Metallosphaera sedula, Acidianus brierleyi, Sulfolobus solfataricus, Sulfolobus tokodaii, Sulfolobus acidocaldaricus, Cenarcheum symbiosum, Archaeoglobus fulgidus, Hyperthermus butylicus, Staphylotthermus marinus, Thermofilum pendens, Ingicoccus hospitalis, Pyrobaculum aerophilum, Pyrobaculum islandicum, Pyrobaculum calidifontis, Pyrobaculum furisous, Pyrobaculum abyssi, Pyrobaculum horykoshii, Haloarcula marismortui, Halobacterium sp. NRC-1, Haloquatratum walsbyi, Halorubrum lacusprofundi or Natromonas pharaonis.

Suitable ACCase subunits include the proteins with the amino acid sequences of SEQ ID Nos. 2, 3, 5, 7, 9 or 11 which may be encoded by the nucleotide sequences of SEQ ID No. 1 from the nucleotide at position 331 to the nucleotide at position 1860, SEQ ID No. 1 from the nucleotide at position 1860 to the nucleotide at position2360, SEQ ID No. 4, SEQ ID No 6, SEQ ID No 8, SEQ ID No 10.

Other suitable subunits of acetyl CoA carboxylase are biotin carboxylase (accC) from Chloroflexus aurantiacus, biotin carboxylase carrier protein (accB) from Chloroflexus aurantiacus, carboxytransferase-α (accA) from Chloroflexus aurantiacus and carboxytransferase-β (accD) from Chloroflexus aurantiacus such as the proteins with the amino acid sequences of SEQ ID Nos. 13, 15, 17 and 19, which may be encoded by the nucleotide sequence of SEQ ID Nos. 12, 14, 16 and 18.

Also suitable are nucleotide sequence encoding the BCCP homologue from Sulfolobus metallicus (SEQ ID No. 46), from Acidianus brierly (SEQ ID No. 47), from Sulfolobus tokodaii str. 7 (SEQ ID No. 48), from Acidianus hospitalis W1 (SEQ ID No. 49), from Metallospheara sedula DSM5348 (SEQ ID No. 50), from Metalospheara cuprina Ar-4 (SEQ ID No. 51) from Sulfolobus acidocaldarius DSM639 (SEQ ID No. 52), from Sulfolobus solfataricus P2 (SEQ ID No. 53), from Sulfolobus solfataricus 98/2 (SEQ ID No. 54), from Sulfolobus islandicus L.S.2.15 (SEQ ID No. 55), from Sulfolobus islandicus M.14.25 (SEQ ID No. 56), from Sulfolobus islandicus Y.N.15.51 (SEQ ID No. 57), from Sulfolobus islandicus REY15A (SEQ ID No. 58), from Aciduliprofundum boonei T469 (SEQ ID No. 59), from Chloroflexus aggregans DSM9485 (SEQ ID No. 60), from Oscillochloris trichoides DG6 (SEQ ID No. 61), from Roseiflexus castenholzii DSM 13941 (SEQ ID No. 62), from Roseiflexus sp. RS-1 (SEQ ID No. 63), from Herpetosiphon aurantiacus ATCC 23779 (SEQ ID No. 64) from Nitrosarchaeum limnia SFB1 (SEQ ID No. 65), from Nitrosopumilis maritimus SCM1 (SEQ ID No. 66), from Group I crenarchea HF4000APKG6D3 (SEQ ID No. 67), from Group I crenarchea HF4000ANIW97P9 (SEQ ID No. 68), from Hippea maritima DSM10411 (SEQ ID No. 69) or from Croceibacter atlanticus HTCC2559 (SEQ ID No. 70); nucleotide sequence encoding the BC homologue from Acidianus hospitalis W1 (SEQ ID No. 71), from Sulfolobus tokodaii str. 7 (SEQ ID No. 72), from Acidianus brierly (SEQ ID No. 73), from Metallospheara sedula DSM5348 (SEQ ID No. 74), from Metalospheara cuprina Ar-4 (SEQ ID No. 75), from Sulfolobus acidocaldarius DSM639 (SEQ ID No. 76), from Sulfolobus islandicus M.16.4 (SEQ ID No. 77), from Sulfolobus islandicus Y.G.57.14 (SEQ ID No. 78), from Sulfolobus solfataricus 98/2 (SEQ ID No. 79), from Sulfolobus islandicus.L.D.8.5 (SEQ ID No. 80), from Sulfolobus islandicus M.14.25 (SEQ ID No. 81), from Sulfolobus islandicus HVE10/4 (SEQ ID No. 82) from Sulfolobus islandicus Y.N.15.51 (SEQ ID No. 83) from Sulfolobus solfataricus P2 (SEQ ID No. 84) from Sulfolobus islandicus.L.S.2.15 (SEQ ID No. 85), from Sulfolobus islandicus REY15A (SEQ ID No. 86), from Chloroflexus aggregans DSM9485 (SEQ ID No. 87), from Oscillochloris trichoides DG6 (SEQ ID No. 88), from Roseiflexus sp. RS-1 (SEQ ID No. 89), from Roseiflexus castenholzii DSM 13941 (SEQ ID No. 90), from Herpetosiphon aurantiacus ATCC 23779 (SEQ ID No. 91), from Nitrosopumilis maritimus SCM1 (SEQ ID No. 92), from Nitrosarchaeum limnia SFB 1 (SEQ ID No. 93), from Group I crenarchea HF4000APKG6D3 (SEQ ID No. 94) or from Group I crenarchea HF4000ANIW97P9 (SEQ ID No. 95); nucleotide sequence encoding the CT homologue from Acidianus hospitalis W1 (SEQ ID No. 96), from Metallospheara sedula DSM5348 (SEQ ID No. 97), from Acidianus brierly (SEQ ID No. 98), from Metalospheara cuprina Ar-4 (SEQ ID No. 99), from Sulfolobus solfataricus 98/2 (SEQ ID No. 100), from Sulfolobus tokodaii str. 7 (SEQ ID No. 101), from Sulfolobus islandicus M.14.25 (SEQ ID No. 102), from Sulfolobus islandicus.L.D.8.5 (SEQ ID No. 103), from Sulfolobus islandicus Y.N.15.51 (SEQ ID No. 104), from Sulfolobus solfataricus P2 (SEQ ID No. 105), from Sulfolobus acidocaldarius DSM639 (SEQ ID No. 106) or from Aciduliprofundum boonei T469 (SEQ ID No. 107); nucleotide sequence encoding the CT homologue from Chloroflexus aggregans DSM9485 (SEQ ID No. 108), from Oscillochloris trichoides DG6 (SEQ ID No. 109), from Roseiflexus sp. RS-1 (SEQ ID No. 110), from Roseiflexus castenholzii DSM 13941 (SEQ ID No. 111), from Amonifex degensii KC4 (SEQ ID No. 112), from Sphaerobacter thermophilus DSM 20475 (SEQ ID No. 113), from Roseiflexus sp. RS-1 (SEQ ID No. 114), from Herpetosiphon aurantiacus ATCC 23779 (SEQ ID No. 115), or from Roseiflexus castenholzii DSM 13941 (SEQ ID No. 116); nucleotide sequence encoding the CT β homologue from Chloroflexus aggregans DSM9485 (SEQ ID No. 117), from Oscillochloris trichoides DG6 (SEQ ID No. 118), from Roseiflexus castenholzii DSM 13941 (SEQ ID No. 119), from Roseiflexus sp. RS-1 (SEQ ID No. 120), from Roseiflexus sp. RS-1 (SEQ ID No.121), from Roseiflexus castenholzii DSM 13941 (SEQ ID No.122), from Herpetosiphon aurantiacus ATCC 23779 (SEQ ID No.123), or from Sphaerobacter thermophilus DSM 20475 (SEQ ID No. 124) or nucleotide sequence encoding the CT homologue from Nitrosopumilis maritimus SCM1 (SEQ ID No. 125), from Nitrosarchaeum limnia SFE31 (SEQ ID No. 126), from Group I crenarchea HF4000APKG6D3 (SEQ ID No. 127) or from Group I crenarchea HF4000ANIW97P9 (SEQ ID No. 128).

Also suitable for the invention are variants of the AcetylCoA-carboxylases or subunits thereof mentioned herein.

The term “variant” is intended to mean substantially similar sequences. Naturally occurring allelic variants can be identified with the use of well-known molecular biology techniques, as, for example, with polymerase chain reaction (PCR) and hybridization techniques as herein outlined. Variant (nucleotide) sequences also include synthetically derived (nucleotide) sequences, such as those generated, for example, by using site-directed mutagenesis. Generally, amino acid sequence variants of ACCase or subunits described herein will have at least 40%, 50%, 60%, to 70%, e.g., preferably 71%, 72%, 73%, 74%, 75%, 76%, 77%, 78%, to 79%, generally at least 80%, e.g., 81% to 84%, at least 85%, e.g., 86%, 87%, 88%, 89%, 90%, 91%, 92%, 93%, 94%, 95%, 96%, 97%, to 98% and 99% sequence identity to the amino acid sequences of the ACCases or subunits described herein, and will retain acetylcoA carboxylase activity (either alone or in combination with other subunits). Generally, nucleotide sequence variants have at least 40%, 50%, 60%, to 70%, e.g., preferably 71%, 72%, 73%, 74%, 75%, 76%, 77%, 78%, to 79%, generally at least 80%, e.g., 81% to 84%, at least 85%, e.g., 86%, 87%, 88%, 89%, 90%, 91%, 92%, 93%, 94%, 95%, 96%, 97%, to 98% and 99% sequence identity to the nucleotide sequences encoding the ACCases or subunits described herein, and the encoded products retain acetylCoA carboxylase activity (either alone or in combination with other subunits).

Variants include, but are not limited to, deletions, additions, substitutions, insertions.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other, is regarded as a position with non-identical residues. The “optimal alignment” of two sequences is found by aligning the two sequences over the entire length according to the Needleman and Wunsch global alignment algorithm (Needleman and Wunsch, 1970, J Mol Biol 48(3):443-53) in The European Molecular Biology Open Software Suite (EMBOSS, Rice et al., 2000, Trends in Genetics 16(6): 276-277; see e.g. http://www.ebi.ac.uk/emboss/align/index.html) using default settings (gap opening penalty=10 (for nucleotides)/10 (for proteins) and gap extension penalty=0.5 (for nucleotides)/0.5 (for proteins)). For nucleotides the default scoring matrix used is EDNAFULL and for proteins the default scoring matrix is EBLOSUM62.

“Stringent hybridization conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tₘ) for the specific sequences at a defined ionic strength and pH. The Tₘ is the temperature (under defined ionic strength and pH) at which 50% of the target sequence hybridizes to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridizations (Northern blots using a probe of e.g. 100 nt) are for example those which include at least one wash in 0.2×SSC at 63° C. for 20 min, or equivalent conditions.

“High stringency conditions” can be provided, for example, by hybridization at 65° C. in an aqueous solution containing 6×SSC (20×SSC contains 3.0 M NaCl, 0.3 M Na-citrate, pH 7.0), 5×Denhardt's (100×Denhardt's contains 2% Ficoll, 2% Polyvinyl pyrollidone, 2% Bovine Serum Albumin), 0.5% sodium dodecyl sulphate (SDS), and 20 μg/ml denaturated carrier DNA (single-stranded fish sperm DNA, with an average length of 120-3000 nucleotides) as non-specific competitor. Following hybridization, high stringency washing may be done in several steps, with a final wash (about 30 min) at the hybridization temperature in 0.2-0.1×SSC, 0.1% SDS.

“Moderate stringency conditions” refers to conditions equivalent to hybridization in the above described solution but at about 60-62° C. Moderate stringency washing may be done at the hybridization temperature in 1×SSC, 0.1% SDS.

“Low stringency” refers to conditions equivalent to hybridization in the above described solution at about 50-52° C. Low stringency washing may be done at the hybridization temperature in 2×SSC, 0.1% SDS. See also Sambrook et al. (1989) and Sambrook and Russell (2001).

Providing suitable ACCases or the subunits thereof to the plastids of the cells may be conveniently achieved by providing the plants with one or more DNA molecules expressing one or more DNA regions coding for subunits of the ACCases operably linked to a plant expressible promoter and optionally, a transcription termination region and/or a polyadenylation region functional in plants. The one or more DNA molecules may either be provided to the nucleus in which case the coding regions should be operably linked to a plastid targeting signal. Alternatively, the one or more DNA molecules may be integrated into the genome of the plastids, whereby the plant expressible promoter is a promoter which is expressible in the plastids of a plant, and the optional termination region is a termination region for plastid transcription. DNA molecules for expression in plastids may comprise one or more coding region, the latter arranged in an operon.

In another embodiment of the invention, the feedback inhibition is prevented by reducing the level of 18:1-CoA and/or 18:1-ACP in the plastids of the plant.

Reduction of the level of 18:1-Coenzyme A or 18:1-Acyl Carrier Protein in the plastids can be achieved by increasing the level of FATA enzyme in plastids of said cell e.g. through overexpression from a chimeric DNA construct.

Example of suitable FAT A encoding DNA regions are a nucleotide sequence encoding the amino acid sequence of SEQ ID No 20, such as a nucleotide sequence of SEQ ID No. 21 or a nucleotide sequence having 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% nucleotide sequence identity therewith

Reduction of the level of 18:1-Coenzyme A or 18:1-Acyl Carrier Protein in the plastids may also be achieved by increasing the level of Acyl-CoA binding proteins in said plant cell. e.g. through overexpression from a chimeric DNA construct.

Example of suitable ACB proteins are the nucleotide sequence encoding the amino acid sequence of SEQ ID No 23 or SEQ ID No. 25, such as a nucleotide sequence of SEQ ID No. 22 from nucleotides 103 to 2109 or the nucleotide sequence of SEQ ID No. 24 from nucleotide 106 to 384 or a nucleotide sequence having 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% nucleotide sequence identity therewith.

As used herein, the term “plant-expressible promoter” means a DNA sequence which is capable of controlling (initiating) transcription in a plant cell. This includes any promoter of plant origin, but also any promoter of non-plant origin which is capable of directing transcription in a plant cell, i.e., certain promoters of viral or bacterial origin such as the CaMV35S (Harpster et al., 1988 Mol. Gen. Genet. 212, 182-190), the subterranean clover virus promoter No 4 or No 7 (WO9606932), or T-DNA gene promoters but also tissue-specific or organ-specific promoters including but not limited to seed-specific promoters (e.g., WO89/03887), organ-primordia specific promoters (An et al., 1996, The Plant Cell 8, 15-30), stem-specific promoters (Keller et al., 1988, EMBO J. 7, 3625-3633), leaf specific promoters (Hudspeth et al., 1989, Plant Mol Biol 12, 579-589), mesophyl-specific promoters (such as the light-inducible Rubisco promoters), root-specific promoters (Keller et al., 1989, Genes Devel. 3, 1639-1646), tuber-specific promoters (Keil et al., 1989, EMBO J. 8, 1323-1330), vascular tissue specific promoters (Peleman et al., 1989, Gene 84, 359-369), stamen-selective promoters (WO89/10396, WO 92/13956), dehiscence zone specific promoters (WO 97/13865) and the like.

Seed specific promoters are well known in the art, including the USP promoter from Vicia faba described in DE10211617; the promoter sequences described in WO2009/073738; promoters from Brassica napus for seed specific gene expression as described in WO2009/077478; the plant seed specific promoters described in US2007/0022502; the plant seed specific promoters described in WO03/014347; the seed specific promoter described in WO2009/125826; the promoters of the omega_{—}3 fatty acid desaturase family described in WO2006/005807 and the like.

The plant-expressible promoter should preferably be a heterologous promoter, i.e. a promoter is not normally associated in its natural context with the coding DNA region operably linked to it in the DNA molecules according to the invention.

A signal peptide is a short (3-60 amino acids long) peptide chain that directs the transport of a protein. Signal peptides may also be called targeting signals, signal sequences, transit peptides, or localization signals. A ‘transit peptide’ used in this system refers to the part of the pre-sequence that targets the protein to other organelles, such as mitochondria, chloroplasts and apoplasts. A plastid transit peptide refers to a transit peptide that targets the protein to plastids. Plastid transit peptide are well known in the art (see e.g. a review by Patron and Waller, 2007 Bioessays, 29(10) 1048-1058.

Suitable chloroplast targeting peptides include the transit peptide of the Arabidopsis thaliana atS1A ribulose 1,5 biphosphate carboxylase small subunit gene (De Almeida et al. (1989). Molecular and General Genetics 218: 78-86; SEQ ID Nos: 38-39) a synthetic chloroplast targeting presequence based on the consensus sequence of dicotyledonous ribulose-1,5-biphosphate carboxylase/oxygenase small subunit chloroplast targeting sequence (Marillonnet et al. (2004) Proceedings National Academy Science 101: 6852-6857; SEQ ID Nos: 40-41) a Brassica codon usage adapted coding sequence of the transit peptide from Solanum tuberosum ribulose-1,5-bisphosphate carboxylase/oxygenase small subunit. (Fritz et al. 1993 Gene, 137(2):271-4; SEQ ID Nos: 42-43), the coding sequence of the optimized transit peptide, containing sequence of the RuBisCO small subunit genes of Zea mays (corn) and Helianthus annuus (sunflower) (Lebrun et al., 1996 U.S. Pat. No. 5,510,471; SEQ ID Nos: 44-45) or the transit peptide from the Ricinus Communis cDNA encoding Δ9-18:0-ACP desaturase (Shanklin et al. 1991 Proc Natl Acad Sci USA. March 15; 88(6):2510-4; SEQ ID Nos: 36-37).

Methods for plastid transformation are known in the art. Maliga, 2004 (Annu Rev Plant Biol. 2004; 55:289-313) provides a review of such methods. Plastid transformation in Brassica's has been described in U.S. Pat. No. 6,891,086, by Nugent et al., 2006 (Plant Science 170(1) 135-142) or by Cheng et al. 2010 (Plant Cell Rep. 29(4) 371-381. Methods for soybean plastid transformation have been described by Dufourmantel et al. 2004, Plant Mol. Biol. 55(4) 479-489.

Plastid expressible promoters are also well known in the art and include the plastid ribosomal RNA operon promoter (Suzuki et al. 2003, Plant Cell, 15, 195-205). Kung and Lin compiled 60 chloroplast promoter sequences from higher plants (1985, Nucl. Acids Res. 11:7543-7549).

Methods to obtain transgenic plants are not deemed critical for the current invention and any transformation method and regeneration suitable for a particular plant species can be used. Such methods are well known in the art and include Agrobacterium-mediated transformation, particle gun delivery, microinjection, electroporation of intact cells, polyethyleneglycol-mediated protoplast transformation, electroporation of protoplasts, liposome-mediated transformation, silicon-whiskers mediated transformation etc. The transformed cells obtained in this way may then be regenerated into mature fertile plants.

The obtained transformed plant can be used in a conventional breeding scheme to produce more transformed plants with the same characteristics or to introduce the chimeric gene according to the invention in other varieties of the same or related plant species, or in hybrid plants. Seeds obtained from the transformed plants contain the chimeric genes of the invention as a stable genomic insert and are also encompassed by the invention.

The methods and means described herein are believed to be suitable for all plant cells and plants, both dicotyledonous and monocotyledonous plant cells and plants including but not limited to cotton, Brassica vegetables, oilseed rape, wheat, corn or maize, barley, sunflowers, rice, oats, sugarcane, soybean, vegetables (including chicory, lettuce, tomato), tobacco, potato, sugarbcet, papaya, pineapple, mango, Arabidopsis thaliana, but also plants used in horticulture, floriculture or forestry. Especially suited are oil producing plants such as rapeseed (Brassica spp.), flax (Linum usitatissimum), safflower (Carthamus tinctorius), sunflower (Helianthus annuus), maize or corn (Zea mays), soybean (Glycine max), mustard (Brassica spp. and Sinapis alba), crambe (Crambe abyssinica), eruca (Eruca sava), oil palm (Elaeis guineeis), cottonseed (Gossypium spp.), groundnut (Arachis hypogaea), coconut (Cocus nucifera), castor bean (Ricinus communis), coriander (Coriandrum sativum), squash (Cucurbita maxima), Brazil nut (Bertholletia excelsa) or jojoba (Simmondsia chinensis) gold-of-pleasure (Camelina sativa), purging nut (Jatropha curcas), Echium spp., calendula (Calendula officinalis), olive (Olea europaea), wheat (Triticum spp.), oat (Avena spp.), rye (Secale cereale), rice (Oryza sativa), Lesquerella spp., Cuphea spp., meadow foam (Limnanthes alba), avocado (Persea Americana), hazelnut (Corylus), sesame (Sesamum indicum), safflower (Carthamus tinctorius), tung tree (Aleurites fordii), poppy (Papaver somniferum) tobacco (Nicotiana spp.).

The methods and means described herein can also be used in algae such as Scenedesmus dimorphus, Euglena gracilis, Phaeodactylum tricornutum, Pleurochrysis carterae, Prymnesium parvum, Tetraselmis chui, Tetraselmis suecica, Isochrysis galbana, Nannochloropsis salina, Botryococcus braunii, Dunaliella tertiolecta, Nannochloris spp. or Spirulina spp.

As used herein, a “Brassica plant” is a plant which belongs to one of the species Brassica napus, Brassica rapa (or campestris), or Brassica juncea. Alternatively, the plant can belong to a species originating from intercrossing of these Brassica species, such as B. napocampestris, or of an artificial crossing of one of these Brassica species with another species of the Cruciferacea. As used herein “oilseed plant” refers to any one of the species Brassica napus, Brassica rapa (or campestris), Brassica carinata, Brassica nigra or Brassica juncea.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region which is functionally or structurally defined, may comprise additional DNA regions etc.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

Throughout the description and Examples, reference is made to the following sequences:
SEQ ID No. 1: nucleotide sequence of the biotin carboxylase (accC) and BCCP (accB) subunits of Metallosphaera sedula.
SEQ ID No. 2: amino acid sequence of accC subunit of Metallosphaera sedula.
SEQ ID No. 3: amino acid sequence of the accB subunit of Metallosphaera sedula.
SEQ ID No. 4: nucleotide sequence of carboxyltransferase (pccB) of Metallosphaera sedula
SEQ ID No. 5: amino acid sequence of carboxyltransferase (pccB) of Metallosphaera sedula
SEQ ID No. 6: nucleotide sequence of BCCP from Cenarchaeum symbiosum.
SEQ ID No. 7: amino acid sequence of BCCP from Cenarchaeum symbiosum
SEQ ID No. 8: nucleotide sequence of biotin carboxylase from Cenarchaeum symbiosum
SEQ ID No. 9: amino acid sequence of biotin carboxylase from Cenarchaeum symbiosum
SEQ ID No. 10: nucleotide sequence of carboxytransferase from Cenarchaeum symbiosum
SEQ ID No. 11: amino acid sequence of carboxytransferase from Cenarchaeum symbiosum
SEQ ID No. 12: nucleotide sequence of biotin carboxylase (accC) from Chloroflexus aurantiacus
SEQ ID No. 13: amino acid sequence of biotin carboxylase (accC) from Chloroflexus aurantiacus
SEQ ID No. 14: nucleotide sequence of BCCP (accB) from Chloroflexus aurantiacus
SEQ ID No. 15: amino acid sequence of BCCP (accB) from Chloroflexus aurantiacus
SEQ ID No. 16: nucleotide sequence of carboxytransferase-α (accA) from Chloroflexus aurantiacus
SEQ ID No. 17: amino acid sequence of carboxytransferase-α (accA) from Chloroflexus aurantiacus
SEQ ID No. 18: nucleotide sequence of carboxytransferase-β (accD) from Chloroflexus aurantiacus
SEQ ID No. 19: amino acid sequence of carboxytransferase-β (accD) from Chloroflexus aurantiacus
SEQ ID No. 20: nucleotide sequence encoding FATA from Ricinus communis
SEQ ID No. 21: amino acid sequence of FATA from Ricinus communis
SEQ ID No. 22: nucleotide sequence of AcetylCoA binding protein ACBP4
SEQ ID No. 23: amino acid sequence of AcetylCoA binding protein ACBP4
SEQ ID No. 24: nucleotide sequence of AcetylCoA binding protein ACBP6
SEQ ID No. 25: amino acid sequence of AcetylCoA binding protein ACBP6
SEQ ID No. 26: forward primer for cloning of B. napus ACP
SEQ ID No. 27: reverse primer for cloning of B. napus ACP
SEQ ID No. 28: forward primer for cloning of B. napus BC
SEQ ID No. 29: reverse primer for cloning of B. napus BC
SEQ ID No. 30: forward primer for cloning of B. napus BCCP
SEQ ID No. 31: reverse primer for cloning of B. napus BCCP
SEQ ID No. 32: forward primer for cloning of B. napus CT-α
SEQ ID No. 33: reverse primer for cloning of B. napus CT-α
SEQ ID No. 34: forward primer for cloning of B. napus CT-β
SEQ ID No. 35: reverse primer for cloning of B. napus CT-β
SEQ ID No. 36: nucleotide sequence of the transit peptide from the Ricinus communis cDNA encoding delta9-18:0-ACP desaturase
SEQ ID No. 37: amino acid sequence of the transit peptide from the Ricinus communis cDNA encoding delta9-18:0-ACP desaturase
SEQ ID No. 38: nucleotide sequence of the transit peptide from the Arabidopsis thaliana atS1A ribulose 1,5 biphosphate carboxylase small subunit
SEQ ID No. 39: amino acid sequence of the transit peptide from the Arabidopsis thaliana atS1A ribulose 1,5 biphosphate carboxylase small subunit
SEQ ID No. 40: nucleotide sequence of a synthetic chloroplast targeting presequence based on ribulose-1,5-biphosphate carboxylase
SEQ ID No. 41: amino acid sequence of a synthetic chloroplast targeting presequence based on ribulose-1,5-biphosphate carboxylase
SEQ ID No. 42: nucleotide sequence of a Brassica codon usage adapted coding sequence of the transit peptide from Solanum tuberosum ribulose-1,5-bisphosphate carboxylase/oxygenase small subunit
SEQ ID No. 43: amino acid sequence of a Brassica codon usage adapted coding sequence of the transit peptide from Solanum tuberosum ribulose-1,5-bisphosphate carboxylase/oxygenase small subunit
SEQ ID No. 44: nucleotide sequence of optimized transit peptide, containing sequence of the RuBisCO small subunit genes of Zea mays (corn) and Helianthus annuus (sunflower)
SEQ ID No. 45: amino acid sequence of optimized transit peptide, containing sequence of the RuBisCO small subunit genes of Zea mays (corn) and Helianthus annuus (sunflower)
SEQ ID No. 46: BCCP homologue from Sulfolobus metallicus
SEQ ID No. 47: BCCP homologue from Acidianus brierly
SEQ ID No. 48: BCCP homologue from Sulfolobus tokodaii str. 7
SEQ ID No. 49: BCCP homologue from Acidianus hospitalis W1
SEQ ID No. 50: BCCP homologue from Metallospheara sedula DSM5348
SEQ ID No. 51: BCCP homologue from Metalospheara cuprina Ar-4
SEQ ID No. 52: BCCP homologue from Sulfolobus acidocaldarius DSM639
SEQ ID No. 53: BCCP homologue from Sulfolobus solfataricus P2
SEQ ID No. 54: BCCP homologue from Sulfolobus solfataricus 98/2
SEQ ID No. 55: BCCP homologue from Sulfolobus islandicus L.S.2.15
SEQ ID No. 56: BCCP homologue from Sulfolobus islandicus M.14.25
SEQ ID No. 57: BCCP homologue from Sulfolobus islandicus Y.N.15.51
SEQ ID No. 58: BCCP homologue from Sulfolobus islandicus REY15A
SEQ ID No. 59: BCCP homologue from Aciduliprofundum boonei T469
SEQ ID No. 60: BCCP homologue from Chloroflexus aggregans DSM9485
SEQ ID No. 61: BCCP homologue from Oscillochloris trichoides DG6
SEQ ID No. 62: BCCP homologue from Roseiflexus castenholzii DSM 13941
SEQ ID No. 63: BCCP homologue from Roseiflexus sp. RS-1
SEQ ID No. 64: BCCP homologue from Herpetosiphon aurantiacus ATCC 23779
SEQ ID No. 65: BCCP homologue from Nitrosarchaeum limnia SFB1
SEQ ID No. 66: BCCP homologue from Nitrosopumilis maritimus SCM1
SEQ ID No. 67: BCCP homologue from Group I crenarchea HF4000APKG6D3
SEQ ID No. 68: BCCP homologue from Group I crenarchea HF4000ANIW97P9
SEQ ID No. 69: BCCP homologue from Hippea maritima DSM10411
SEQ ID No. 70: BCCP homologue from Croceibacter atlanticus HTCC2559
SEQ ID No. 71: BC homologue from Acidianus hospitalis W1
SEQ ID No. 72: BC homologue from Sulfolobus tokodaii str. 7
SEQ ID No. 73: BC homologue from Acidianus brierly
SEQ ID No. 74: BC homologue from Metallospheara sedula DSM5348
SEQ ID No. 75: BC homologue from Metalospheara cuprina Ar-4
SEQ ID No. 76: BC homologue from Sulfolobus acidocaldarius DSM639
SEQ ID No. 77: BC homologue from Sulfolobus islandicus M.16.4
SEQ ID No. 78: BC homologue from Sulfolobus islandicus Y.G.57.14
SEQ ID No. 79: BC homologue from Sulfolobus solfataricus 98/2
SEQ ID No. 80: BC homologue from Sulfolobus islandicus.L.D.8.5
SEQ ID No. 81: BC homologue from Sulfolobus islandicus HVE10/4
SEQ ID No. 82: BC homologue from Sulfolobus islandicus M.16.4
SEQ ID No. 83: BC homologue from Sulfolobus islandicus Y.N.15.51
SEQ ID No. 84: BC homologue from Sulfolobus solfataricus P2
SEQ ID No. 85: BC homologue from Sulfolobus islandicus.L.S.2.15
SEQ ID No. 86: BC homologue from Sulfolobus islandicus REY15A
SEQ ID No. 87: BC homologue from Chloroflexus aggregans DSM9485
SEQ ID No. 88: BC homologue from Oscillochloris trichoides DG6
SEQ ID No. 89: BC homologue from Roseiflexus sp. RS-1
SEQ ID No. 90: BC homologue from Roseiflexus castenholzii DSM 13941
SEQ ID No. 91: BC homologue from Herpetosiphon aurantiacus ATCC 23779
SEQ ID No. 92: BC homologue from Nitrosopumilis maritimus SCM1
SEQ ID No. 93: BC homologue from Nitrosarchaeum limnia SFB 1
SEQ ID No. 94: BC homologue from Group I crenarchea HF4000APKG6D3
SEQ ID No. 95: BC homologue from Group I crenarchea HF4000ANIW97P9
SEQ ID No. 96: CT homologue from Acidianus hospitalis W1
SEQ ID No. 97: CT homologue from Metallospheara sedula DSM5348
SEQ ID No. 98: CT homologue from Acidianus brierly
SEQ ID No. 99: CT homologue from Metalospheara cuprina Ar-4
SEQ ID No. 100: CT homologue from Sulfolobus solfataricus 98/2
SEQ ID No. 101: CT homologue from Sulfolobus tokodaii str. 7
SEQ ID No. 102: CT homologue from Sulfolobus islandicus M.14.25
SEQ ID No. 103: CT homologue from Sulfolobus islandicus.L.D.8.5
SEQ ID No. 104: CT homologue from Sulfolobus islandicus Y.N.15.51
SEQ ID No. 105: CT homologue from Sulfolobus solfataricus P2
SEQ ID No. 106: CT homologue from Sulfolobus acidocaldarius DSM639
SEQ ID No. 107: CT homologue from Aciduliprofundum boonei T469
SEQ ID No. 108: CT α homologue from Chloroflexus aggregans DSM9485
SEQ ID No. 109: CT α homologue from Oscillochloris trichoides DG6
SEQ ID No. 110: CT α homologue from Roseiflexus sp. RS-1
SEQ ID No. 111: CT α homologue from Roseiflexus castenholzii DSM 13941
SEQ ID No. 112: CT α homologue from Amonifex degensii KC4
SEQ ID No. 113: CT α homologue from Sphaerobacter thermophilus DSM 20475
SEQ ID No. 114: CT α homologue from Roseiflexus sp. RS-1
SEQ ID No. 115: CT α homologue from Herpetosiphon aurantiacus ATCC 23779
SEQ ID No. 116: CT α homologue from Roseiflexus castenholzii DSM 13941
SEQ ID No. 117: CT β homologue from Chloroflexus aggregans DSM9485
SEQ ID No. 118: CT β homologue from Oscillochloris trichoides DG6
SEQ ID No. 119: CT β homologue from Roseiflexus castenholzii DSM 13941
SEQ ID No. 120: CT β homologue from Roseiflexus sp. RS-1
SEQ ID No.121: CT β homologue from Roseiflexus sp. RS-1
SEQ ID No.122: CT β homologue from Roseiflexus castenholzii DSM 13941
SEQ ID No.123: CT β homologue from Herpetosiphon aurantiacus ATCC 23779
SEQ ID No. 124: CT β homologue from Sphaerobacter thermophilus DSM 20475
SEQ ID No. 125: CT homologue from Nitrosopumilis maritimus SCM1
SEQ ID No. 126: CT homologue from Nitrosarchaeum limnia SFB 1
SEQ ID No. 127: CT homologue from Group I crenarchea HF4000APKG6D3
SEQ ID No. 128: CT homologue from Group I crenarchea HF4000ANIW97P9
SEQ ID No. 129: amino acid sequence of the Biotin Carboxyl Carrier Protein from Cenarchaeum symbiosum with N-terminal linked chloroplast protein from Ricinus communis stearoyl-ACP desaturase.
SEQ ID No. 130: nucleotide sequence of the Biotin Carboxyl Carrier Protein from Cenarchaeum symbiosum with N-terminal linked chloroplast protein from Ricinus communis stearoyl-ACP desaturase (codon optimized for expression in Arabidopsis thaliana).
SEQ ID No. 131: amino acid sequence of the Biotin Carboxylase from Cenarchaeum symbiosum with N-terminal linked chloroplast protein from Ricinus communis stearoyl-ACP desaturase.
SEQ ID No. 132: nucleotide sequence of the Biotin Carboxylase from Cenarchaeum symbiosum with N-terminal linked chloroplast protein from Ricinus communis stearoyl-ACP desaturase (codon optimized for expression in Arabidopsis thaliana).
SEQ ID No. 133: amino acid sequence of the Carboxyltransferase from Cenarchaeum symbiosum with N-terminal linked chloroplast protein from Ricinus communis stearoyl-ACP desaturase.
SEQ ID No. 134: nucleotide sequence of the Carboxyltransferase from Cenarchaeum symbiosum with N-terminal linked chloroplast protein from Ricinus communis stearoyl-ACP desaturase (codon optimized for expression in Arabidopsis thaliana).

### EXAMPLES

### Example 1

### Experimental Procedures

### Cell Culture Growth and Analysis

Brassica napus cv Jet Neuf suspension cell cultures were grown in NLN medium (Lichter 1982, Z. Pflanzenphysiol, 105, 427-434) with modifications (Shi et al. 2008, Plant Cell Tiss Org, 92 131-139). Cells were grown shaking at 160 rpm at 25° C. in either 50 mL or 100 mL volumes (in 125 mL or 250 mL flasks, respectively) under constant fluorescent light at 50 μmol m⁻¹ s⁻¹. Medium was refreshed every 48 h for experiments lasting longer than two days. Tween-esters were obtained from Sigma (St. Louis, Mo. USA). A 150 mM stock solution was made by dissolving 9.8 g in 50 mL of water and it was filter sterilized before addition to cultures. Subculturing was done every eight days and new cultures were inoculated with about 200 mg of cells. Cells were harvested by filtering with a Buchner funnel, were rinsed three times with distilled water, and were frozen immediately in liquid N2 in preweighed aluminum foil pouches. Dry weight to fresh weight ratio was determined by lyophilizing a known fresh weight of cells. For SDS-PAGE, proteins were extracted in 3 volumes (w/v) of 50 mM Tris-Cl, pH 7.5, 10 mM KCl, 5 mM MgCl₂, 1 mM EDTA, 1 mM DTT, 0.1% Triton X-100, and were quantified by Bradford assay.

### Lipid Extraction and Quantification

Lipids were extracted from up to 100 mg fresh weight of frozen cells by homogenizing twice in 500 μL of methanol:chloroform:formic acid (20:10:1 v/v) using glass beads. The combined organic solvent was extracted with 500 μL of 1 M KCl, 0.2 M H₃PO₄ and the organic phase was recovered, dried under N₂, and resuspended in hexane. Lipid classes were separated by TLC using silica gel G TLC Uniplates (Analtech, Newark, Del., USA) with hexane:diethylether:acetic acid (80:20:1, v/v) for neutral lipids or with acetone:toluene:water (91:30:7 v/v) with 0.15 M ammonium sulfate impregnated plates for polar lipids. Loading was equivalent to 10 or 20 mg fresh weight. Lipids were visualized with iodine vapor.

Fatty acid quantification was done by analysis of fatty acid methyl esters (FAMEs). FAMEs were prepared by incubation of 17:0 internal standard and lipid extracts or silica powder scraped from TLC plates in 1 mL of 12% (w/w) BCl₃ in methanol for 1 h at 85° C., extracting them with 1 mL of water and 2 mL hexane and then drying under N₂. FAMEs resuspended in hexane were analyzed with an HP6890 gas chromatograph-flame ionization detector (Agilent Technologies) or an HP5890 gas chromatograph-mass spectrometer (Hewlett-Packard) fitted with 60 m×250 μm SP-2340 capillary columns (Supelco). Helium flow rate was 1.1 mL min⁻¹ and oven temperature started at 100° C., increased at 15° C. min⁻¹ to 240° C., and held at that temperature for 5 minutes. Mass spectrometry was performed with an HP5973 mass selective detector (Hewlett-Packard).

### 1-¹³C-Oleoyl-Tween Synthesis

1-¹³C-oleic acid was obtained from Cambridge Isotope Laboratories (Andover, Mass., USA). The custom Tween-ester was synthesized by reacting acyl chloride with Tween backbone. Tween backbone was synthesized (Terzaghi 1986, Plant Physiol, 82, 771-779) and purified (Wisnieski et al. 1973, Proc Natl. Acad Sci USA, 70, 3669-3673) as previously described. The acyl chloride was prepared by first suspending 350 mg of 1-¹³C-oleic acid in 10 mL CH₂Cl₂, This solution was chilled on ice, dried under argon, and reacted with 2.5 molar equivalents of oxalyl chloride. DMF was added dropwise (5-10 drops) over 30 min until CO and CO₂ no longer bubbled from the solution. Excess oxalyl chloride was removed under vacuum and the acyl chloride was suspended in 8 mL CH₂Cl₂. About 25 mg of 4-dimethylaminopyridine and 750 μL of N,N-diisopropylethylamine were dissolved in 2 mL of CH₂Cl₂ and were added to 1 g of Tween backbone dissolved in 8 mL CH₂Cl₂. Acyl chloride was added to dissolved Tween backbone and the reaction was stirred for 24 h at 25° C. Tween-esters were purified by chromatography and were verified by GC-MS and NMR. 1-¹³C-oleoyl-Tween was suspended in water, filter sterilized, and added to culture medium as was Tween-80. Lipid extraction and GC/MS was done as described above. ¹³C-fatty acids were detected and quantified and corrected for natural isotope abundance as previously described (Schwender et al. 2003, J Biol Chem, 278, 29442-29453).

### ¹⁴C Labeling

All radioisotopes were obtained from American Radiolabeled Chemicals (St. Louis, Mo. USA). Labeling was conducted on cells 5 days after subculturing with a culture density of ˜20 mg FW mL⁻¹ and that had medium refreshed 16 h prior. For labeling, three 1 mL aliquots of cells were carefully removed from each flask and were labeled with either 0.2 μCi of 1,2-¹⁴C-acetate (50-60 mCi mmol⁻¹) or 2-¹⁴C-malonate (40-60 mCi mmol⁻¹) for 15 minutes at 25° C. with occasional shaking. Haloxyfop (Sigma) was dissolved in DMSO and added 30 min prior to labeling. Lipids were extracted and separated by TLC as described above. Radioactivity was detected by phosphorimaging and was quantified using ImageQuant software (GE Healthcare, Piscataway, N.J., USA). Incorporation of label into individual fatty acids was determined by making FAMEs as described above, separating the methyl esters by TLC as described in (Koo, Fulda, Browse and Ohlrogge 2005, Plant J, 44, 620-632) and measuring radioactivity by phosphorimaging.

### Free Fatty Acid, Acyl-CoA, and Acyl-ACP Extraction

Free fatty acids were extracted from tissue by quenching ˜300 mg of frozen cells in 2 mL boiling isopropanol for 5 min. Once cooled, 2 mL of 0.9% NaCl was added and lipids were extracted twice with 4 mL of hexane. Neutral lipids were separated by TLC and free fatty acids were scraped, made into FAMEs, and analyzed by GC-FID or GC-MS as described above. Acyl-CoAs were extracted from ˜15 mg FW of cells and quantified as previously described (Larson and Graham 2001, Plant J, 25, 115-125). Acyl-ACPs were extracted and quantified as previously described (Kopka, Ohlrogge and Jaworski 1995, Anal Biochem, 224, 51-60) with the following modifications: 1) Internal standards used were 11:0-CoA (Sigma) and 17:0—ACP made from Spinach ACP as previously described (Broadwater and Fox 1999, Protein Expr Purif, 15, 314-326). 2) Fully dissolving TCA precipitated proteins required three extractions with MOPS buffer rather than just one.

### Enzyme Assays

All chemicals were obtained from Sigma and radioisotopes from American Radiolabeled Chemicals. ACCase activity was measured as the acetyl-CoA dependent incorporation of ¹⁴C—NaHC0₃ into acid and stable products. Crude cell extracts were prepared by grinding fresh cells in 3 volumes (w/v) of 50 mM Tris-Cl, pH 7.5, 100 mM KCl, 5 mM MgCl₂, 1 mM DTT, 0.1% TritonX-100, 10% glycerol, and plant protease inhibitor cocktail (Sigma). Homogenate was gently mixed on ice for 10 min and then centrifuged for 5 min at 3000 g. Desalting of extracts was done with PD-10 columns (GE Life Sciences). Assays conditions were as previously described (Thelen and Ohlrogge 2002, Arch Biochem Biophys, 400, 245-257). Reactions were initiated by the addition of 5 μlL cell extract and were stopped by the addition of 15 μL 12 N HCL. The contents were dried completely at 55° C. and the solids were resuspended in 30 μL water and counted by liquid scintillation spectroscopy. Minus acetyl-CoA controls were always included. Assays went for 30 min, except when measuring the effects of metabolites (10 min). Metabolites were added to the reaction immediately after cell extract to preserve labile thioester bonds. FFA stock solutions were made in ethanol and acyl-CoAs were suspended in water. Acyl-ACP used was made as described for spinach ACP (Broadwater and Fox 1999, Protein Expr Purif, 15, 314-326) except that BnACP (GenBank:X13127.1) was used instead. The ACP cDNA was cloned from cell cultures using the following primers: F-GCGGCCAAACCAGAGACG (SEQ ID No. 26) and R-TCAGTGGTGGTGGTGGTGGTGCTTCTTGGCTTGCACCAGCTCT (SEQ ID No. 27) incorporating a 6× his-tag. Acyl-ACP thioesterase assays were conducted on crude cell extracts as previously described (Eccleston and Ohlrogge 1998, Plant Cell, 10, 613-622), except that assay buffer was the same as for ACCase assays.

qPCR Analysis

RNA was isolated from cultured cells using Trizol Reagent (Invitrogen, Carlsbad, Calif., USA) and was treated with DNase. cDNA was synthesized from 1 μg of total RNA using the Bio-Rad (Hercules, Calif., USA) iScript cDNA synthesis kit. qPCR was performed with 1:50 of the cDNA product and SSO Fast EvaGreen supermix (Bio-Rad). Reference genes were ACT7 and UBC21 using primers as described previously (Chen et al. 2010 Anal Biochem, 405, 138-140). Primers for experimental genes were as follows: BC, biotin carboxylase (GenBank:A Y034410.1), F-TTGGTGAAGCTCCTAGCAACCAGT (SEQ ID No. 28) and R-TTCTTCATCGTCTCCCTGGCAGTT (SEQ ID No. 29); BCCP, biotin carboxyl carrier protein (GenBank: X90730.1), F-AGTGACTAACGGTGGGTGCTTGAA (SEQ ID No. 30), R-TGATAAACTGGAGCTGGTGGTGGT (SEQ ID No. 31); CT-α, carboxytransferase-α (GenBank GQ341624.1), F-TACGTGACAGCTCGCCTCAAGAAA (SEQ ID No. 32), R-CAAACCAGTTTCAGCCGCCATCTT (SEQ ID No. 33); CT-β, carboxytransferase-β (GenBank: Z50868.1), F-GGAGCACGAATGCAAGAAGGAAGT (SEQ ID No. 34), R-ACATACCCAAACTTGCTGTCACCC (SEQ ID No. 35). Relative expression level was calculated using REST software (Qiagen, Valencia, Calif., USA).

### Example 2

### Establishing Conditions for Fatty Acid Feeding to B. napus Suspension Cells

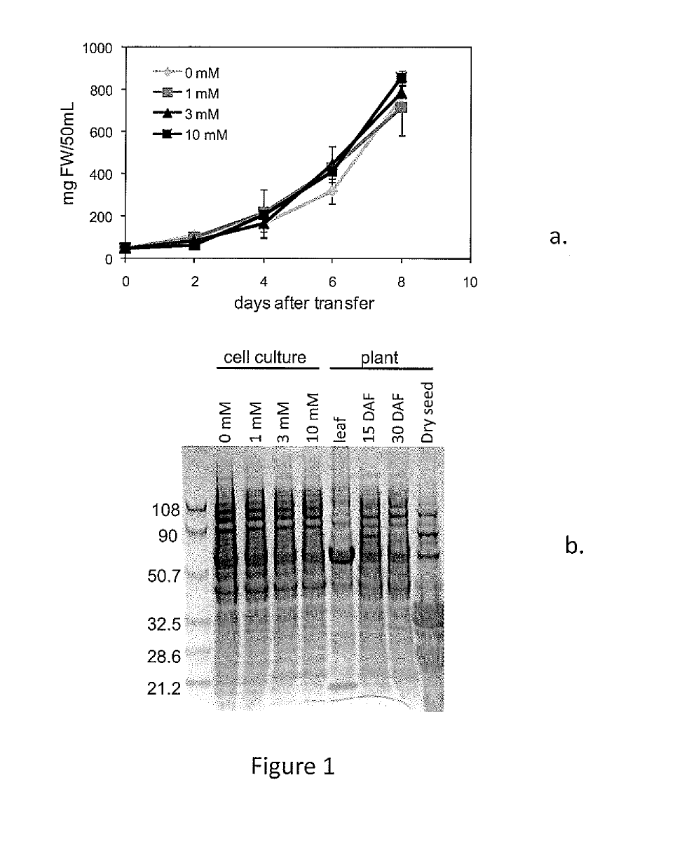

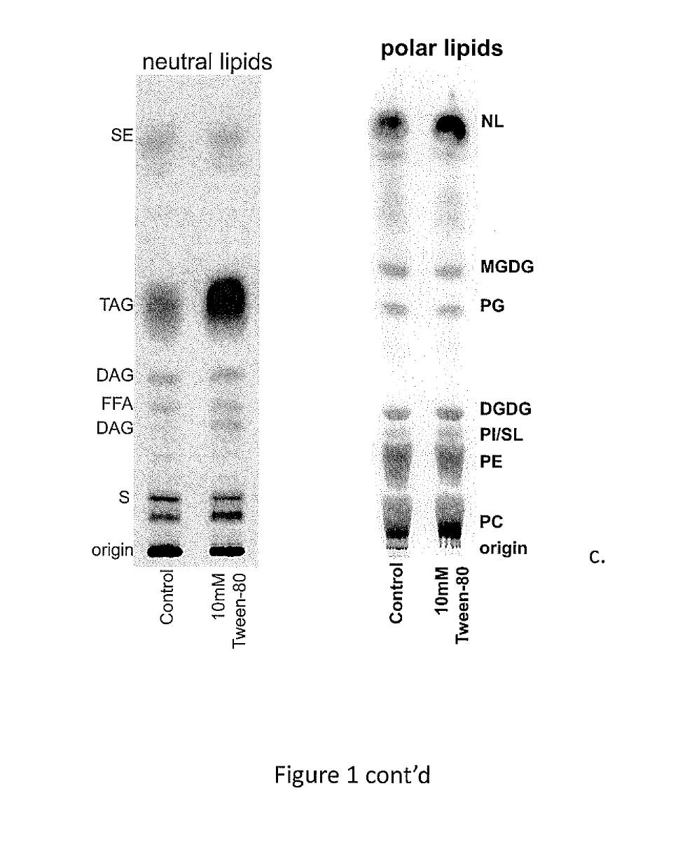

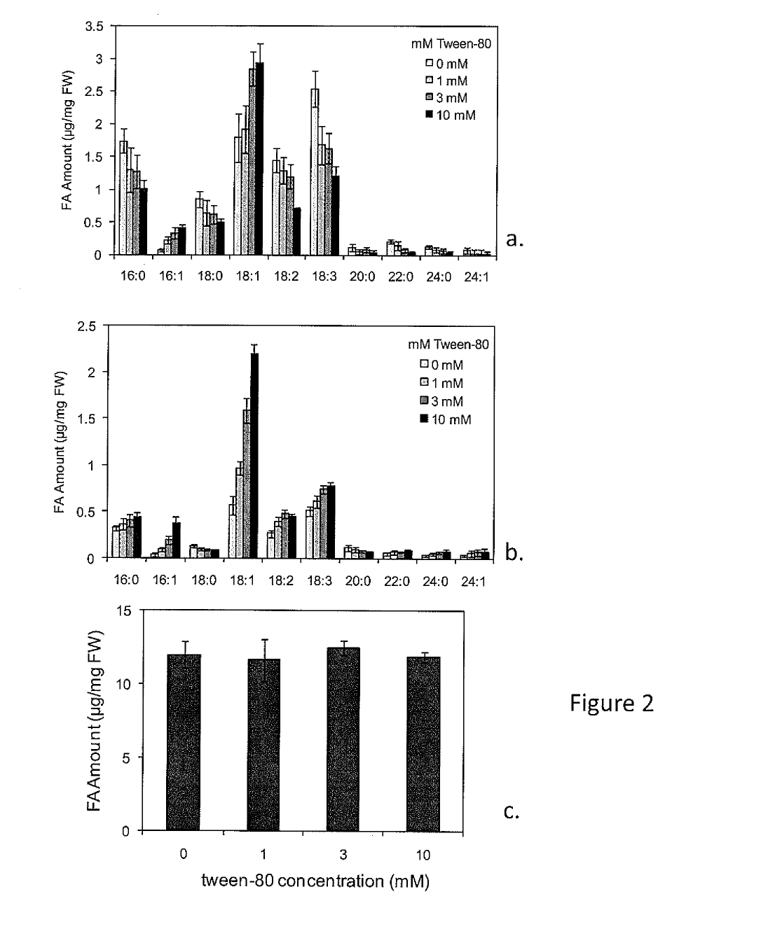

In order to study the feedback regulation of fatty acid synthesis it was necessary to first establish conditions where fatty acids could be fed while minimizing any negative pleiotropic effects. Commercial Tween-80, containing predominately oleic acid (18:1), had no effect on growth rate when added at concentrations up to 10 mM (FIG. 1a). Protein composition of the cells, which was very similar to that of a developing embryo, appeared unaffected after eight days of Tween-80 feeding (FIG. 1b). Water content of the cells was also unaffected; the fresh weight to dry weight ratio being 16.2±0.4 for control and 16.0±0.5 for cells fed 10 mM Tween:-80. As reported for soybean cell cultures (Terzaghi 1986, Plant Physiol, 82, 771-779), the fatty acid composition of B. napus cells was altered by Tween-80. Polar lipids and TAG from cells fed Tween-80 for eight days were quantified and the results are shown in FIG. 2. In both lipid classes, 18:1 accumulated to a level dependent on Tween-80 concentration. For polar lipids (i.e. membrane lipids) this was accompanied by decreases in the amounts of all other fatty acids, except palmitoleic (16:1) which is a minor component of commercial Tween-80 (FIG. 2a). On the other hand, all fatty acids remained constant or increased in TAG (FIG. 2b). Despite these changes, total fatty acid amount remained constant (FIG. 2c) and, with the exception of TAG, polar and neutral lipid profiles appeared unaltered (FIG. 1c).

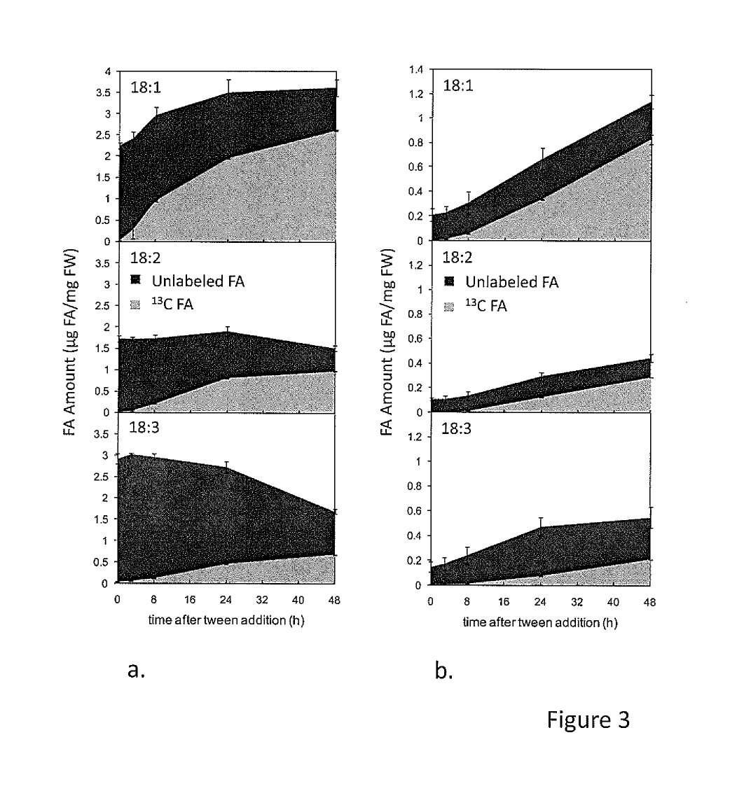

To quantify the contribution of fatty acids from Tween-80 to the changes in fatty acid composition we synthesized 1-¹³C-oleoyl-Tween and performed a time course experiment in which exogenous fatty acids could be distinguished by the presence of the ¹³C isotope (FIG. 3). After just three hours in the presence of 10 mM ¹³C-oleoyl-Tween, ¹³C-18:1 constituted about 10% of the total 18:1, indicating rapid uptake and incorporation. As time progressed, label appeared in desaturation products (18:2 and 18:3) and by 48 hours the proportion of ¹³C-label increased to about 70% of 18-carbon fatty acids. Label was not detected in any other fatty acids, including elongation products. In the case of TAG, increases in individual fatty acid amounts were mostly accounted for by uptake and metabolism of ¹³C-18:1 (FIG. 3b). For polar lipids, ¹³C-18:1 not only accounted for increased 18:1 amount, but actually replaced endogenous fatty acids. This is evident from the fact that unlabeled fatty acids decreased while ¹³C-label increased (FIG. 3a). Such an effect could be due to either increased turnover or reduced synthesis of de novo fatty acids. The effect seems specific to unlabeled (t. e. de novo) fatty acids, indicating reduced synthesis.

### Example 3

### Plastidic ACCase is Reversibly Inhibited in Response to Tween-80

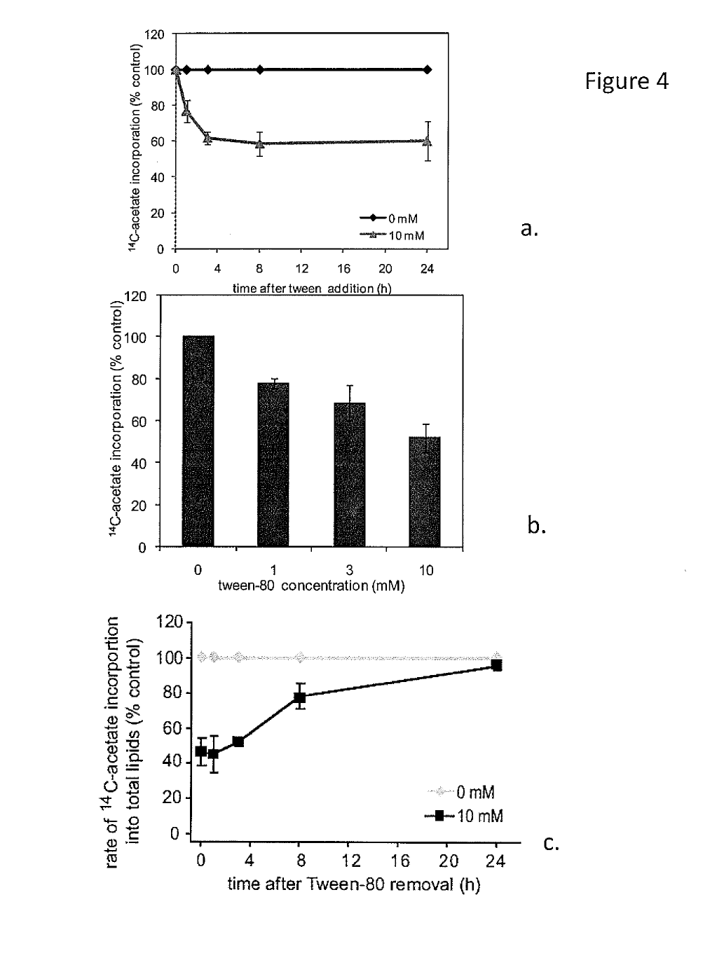

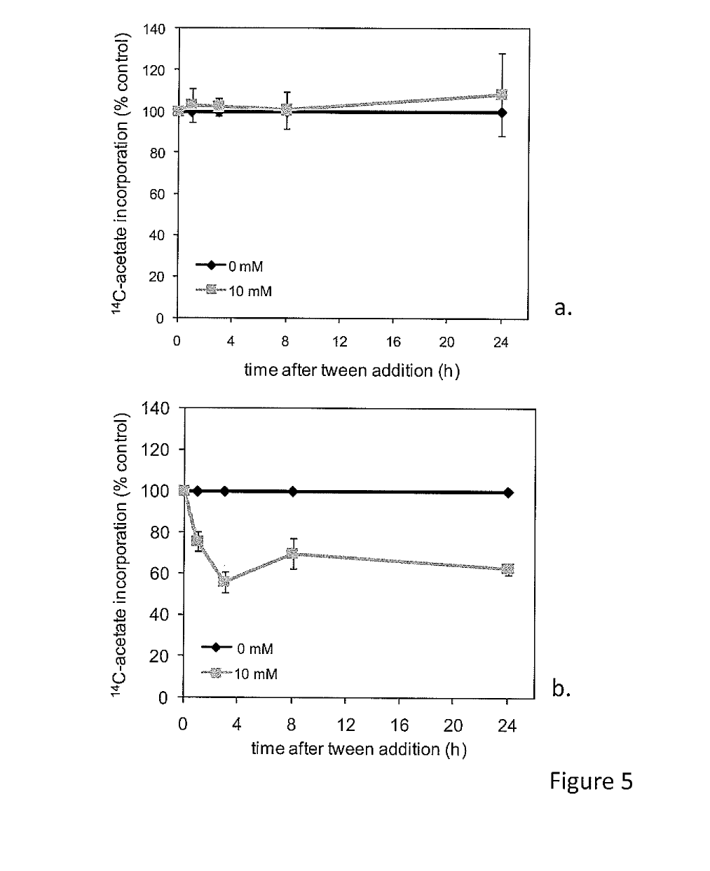

Feedback inhibition of fatty acid synthesis was measured by the addition of a ¹⁴C-acetate tracer. FIG. 4a shows that, indeed, the rate of ¹⁴C-acetate incorporation into lipids is reduced by 40% as soon as three hours after the addition of 10 mM Tween-80 to the medium. Sterol synthesis is dependent on acetyl-CoA and ATP, both of which are required for fatty acid synthesis. Therefore, if fatty acid synthesis is inhibited due to substrate limitation; so should be sterol biosynthesis. ¹⁴C-acetate incorporation into sterols was unaffected by Tween-80 feeding, while incorporation into free fatty acids mirrored that of total lipids (FIG. 5), showing that the effect is restricted to fatty acid synthesis. We found that the degree of feedback inhibition was dependent on the concentration of Tween-80 in the medium (FIG. 4b) and was completely reversible after its removal (FIG. 4c). These results are consistent with a biochemical mode of action for feedback inhibition. Three hour time points were used for the remainder of this study to separate the period of maximum feedback inhibition from any lipid homeostatic mechanisms arising from the gross fatty acid compositional changes caused by long term Tween-80 feeding.

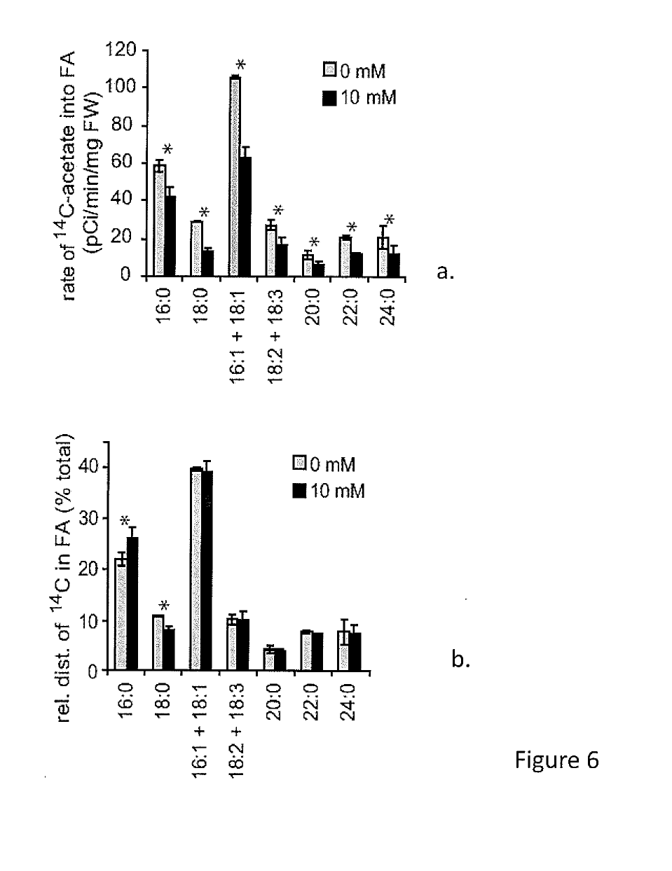

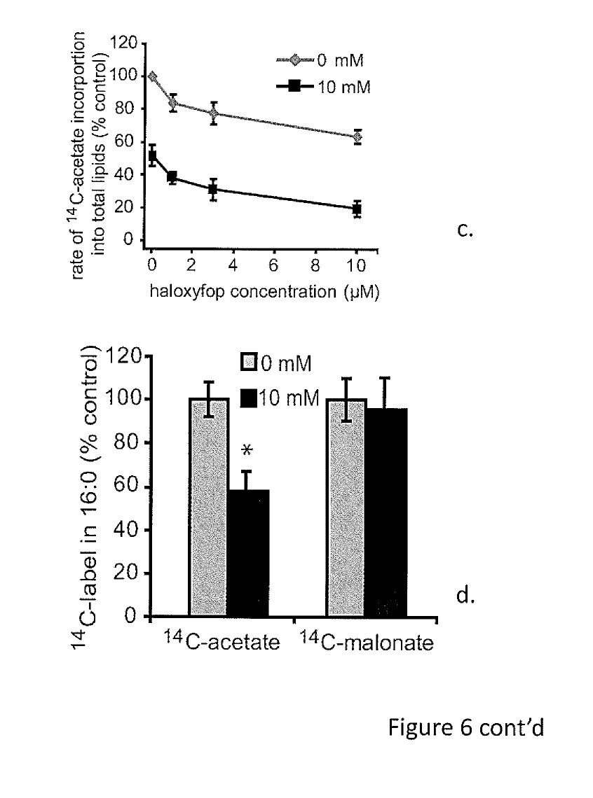

Plants contain plastidic and cytosolic ACCase and F AS enzyme systems, both of which are capable of incorporating ¹⁴C-acetate into fatty acids. Comparing the distribution of label in individual fatty acid species can therefore provide information on the relative contribution of these pathways. Tween-80 feeding, while reducing incorporation of ¹⁴C-acetate into fatty acids by 40% (FIG. 6a), did not affect the labeling pattern (FIG. 6b). This is consistent with reduced de nova fatty acid synthesis inside the plastid, rather than reduced cytosolic elongation which would have resulted in a higher proportion of label in long chain fatty acids. ¹⁴C-acetate labeling was also conducted in the presence of haloxyfop, a specific inhibitor of multifunctional (cytosolic) ACCase. The degree to which ¹⁴C-acetate incorporation is inhibited by haloxyfop is proportional to cytosolic elongation activity, whereas haloxyfop-resistant incorporation is from the de nova pathway inside the plastid. FIG. 6c shows that haloxyfop inhibited ¹⁴C-acetate incorporation by the same amount in cultures with or without Tween-80, as evidenced by the fact that the inhibition curves parallel one another. However, haloxyfopresistant incorporation (represented by the area below the curves) was reduced by about half in cultures with 10 mM Tween-80, showing that Tween-80 feeding specifically causes inhibition of de nova fatty acid synthesis in the plastid.

These data show that ACCase or some downstream component of FAS in the plastid is inhibited upon the addition of Tween-80. To separate these possibilities, we exploited the fact that exogenous malonate can be converted to malonyl-CoA and used by FAS, bypassing the ACCase reaction (Kannangara et al. 1973, Plant Physiol, 52, 156-161). If ACCase is the only point of inhibition then the rate of ¹⁴C-malonate labeling of de novo fatty acids should be the same in cultures with or without Tween-80. FIG. 6d shows that incorporation of ¹⁴C-malonate into 16 and 18 carbon fatty acids was not inhibited by Tween-80 as compared to ¹⁴C-acetate labeled controls. Therefore ACCase, and not F AS, is inhibited by Tween-80 feeding. When combined, the data from FIG. 6 unambiguously identify plastidic ACCase as the target of Tween-80 induced feedback inhibition.

### Example 4

### ACCase Activity and Message are not Reduced in Tween Fed Cells

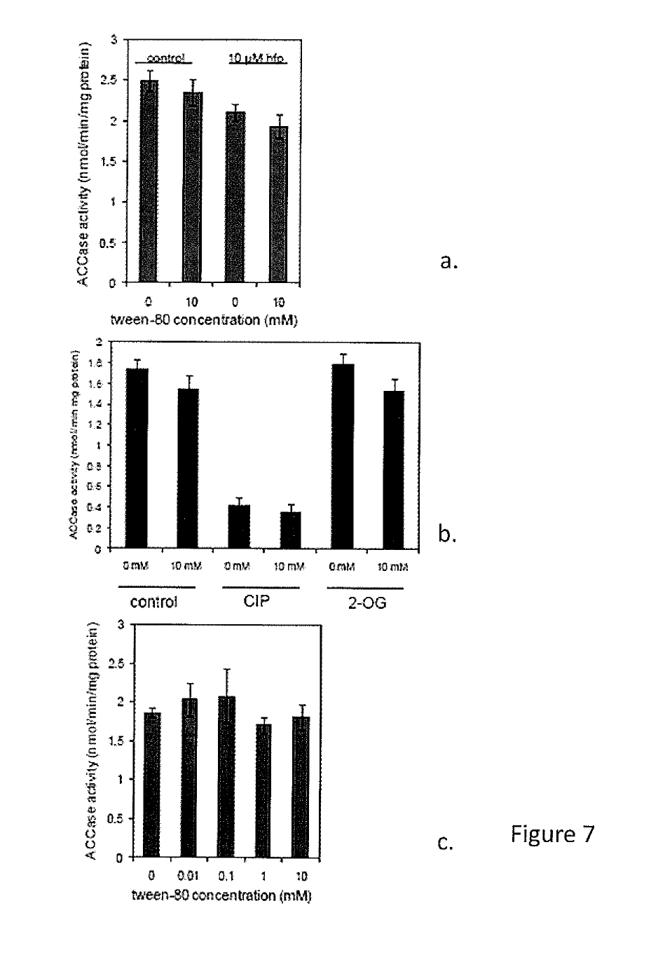

Gene expression analysis and enzyme assays were performed to better understand the apparent reduction in ACCase activity during Tween-80 feeding. Quantitative real-time PCR was used to measure the expression of genes encoding plastidic ACCase subunits. The specific genes selected for analysis were those originally identified as being embryo expressed in Brassica napus plants (Elborough et al. 1996, Biochemical Journal, 315, 103-112). Table 1 shows that expression of all four genes is unaffected by a three hour Tween-80 treatment. Additionally, measurement of ACCase activity from crude extracts of the same cells revealed that maximum ACCase activity was largely unaffected (Table 1). Desalted extracts gave the same result. Treatment with haloxyfop resulted in about 15% reduction in activity, indicating that plastidic ACCase is dominant in these assays (FIG. 7a). Inactivation by dephosphorylation (Savage and Ohlrogge 1999, Plant J, 18, 521-527) or inhibition by a 2-oxoglutarate dependent interaction with PH protein (Feria Bourrellier et al. 2010, Proc Natl Acad Sci USA, 107, 502-507) have been suggested as other means of regulating ACCase activity. If dephosphorylation is involved in feedback, then treatment with phosphatase would be expected to reduce ACCase activity from inhibited cells to a lesser extent than from the control. If PII interaction is involved, then it is possible that incubation in the presence of 2-oxoglutarate would inhibit ACCase from a Tween-fed extract more than the control. ACCase assays following phosphatase treatment of crude extracts or in the presence of 5 mM 2-oxoglutarate revealed no differences between control and Tween-80 fed cultures (FIG. 7b), which is consistent with these mechanisms having little or no role in the Tween-80 induced feedback. Together, these results indicate that reduction in plastidic ACCase transcript or protein amount and known post-translational mechanisms do not account for feedback inhibition observed under Tween-80 feeding.

TABLE 1
Gene expression and ACCase activity in response to Tween-80.

| | Relative gene expression | | | | ACCase activity |
|---|---|---|---|---|---|
| [Tween-80] | BC | BCCP | CT-α | CT-β | (nmol min⁻¹ mg⁻¹) |
| 0 mM | 1 | 1 | 1 | 1 | 1.88 ± 0.08 |
| 10 mM | 0.91 ± 0.17 | 1.32 ± 0.58 | 1.02 ± 0.33 | 1.18 ± 0.29 | 1.73 ± 0.10^{a} |

Values for gene expression are the mean of three repeats ± SE as calculated with REST software and all p > 0.1. For ACCase activity values are the mean of four repeats ± SD.
^{a}p = 0.96. BC, biotin carboxylase. BCCP, biotin carboxyl carrier protein. CT, carboxytransferase.

### Example 5

### ACCase is Inhibited by 18:1-Containing Tweens

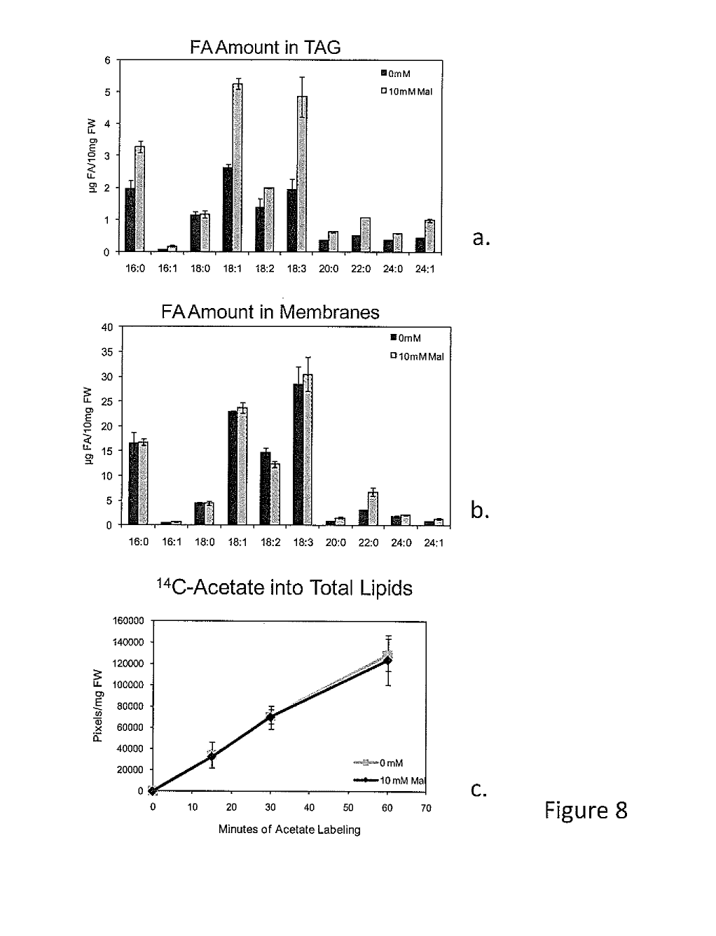

Commercial Tween-80 contains a mixture of fatty acids. To dissect the effects of the individual components, a variety of Tween-esters were tested for their effect on fatty acid synthesis. The compositions of individual Tweens are listed in Table 2 along with results from ¹⁴C-acetate labeling experiments. Tween-40 and Tween-60 containing only saturated fatty acids did not inhibit acetate labeling of lipids to the same extent as Tween-80 or -85 containing primarily 18:1. Custom synthesized Tween-18:1 also produced maximum inhibition. In addition, malonate feeding, which stimulates fatty acid production (most likely) by feeding into the malonyl-CoA pool, does not cause inhibition of ¹⁴C-acetate incorporation (FIG. 8), suggesting that ACCase is unlikely to be inhibited by malonyl-CoA in vivo. Taken together, these results point to 18:1 or a downstream metabolite as being the cause of feedback inhibition.

TABLE 2
Inhibition of ¹⁴C-acetate labeling of lipids by various Tweens.

| | | | | | | Relative |
|---|---|---|---|---|---|---|
| | Fatty acid composition of | | | | | ¹⁴C-acetate |
| | Tween-esters (mol %) | | | | | incorporation |
| | 14:0 | 16:0 | 16:1 | 18:0 | 18:1 | (% control) |
| Tween-40 | 2.3 | 94.0 | n.d | 3.7 | n.d | 84.4 ± 3.4 |
| Tween-60 | 2.8 | 45.4 | n.d | 51.8 | n.d | 85.9 ± 3.4 |
| Tween-80 | 1.9 | 7.3 | 2.4 | 2.1 | 86.4 | 50.9 ± 7.0 |
| Tween-85 | 2.7 | 5.4 | 6.7 | 2.2 | 83.0 | 48.8 ± 5.9 |
| Tween-18:1 | n.d. | n.d. | n.d | n.d | 100 | 51.5 ± 6.7 |

Values are the mean of three repeats ± SD where included. ¹⁴C acetate labeling is relative to a control sample without any Tweens.

### Example 6

### Mechanism of Tween-80 Inhibition

Tween-fatty acid esters enter cells and are hydrolyzed to yield free fatty acids (Terzaghi

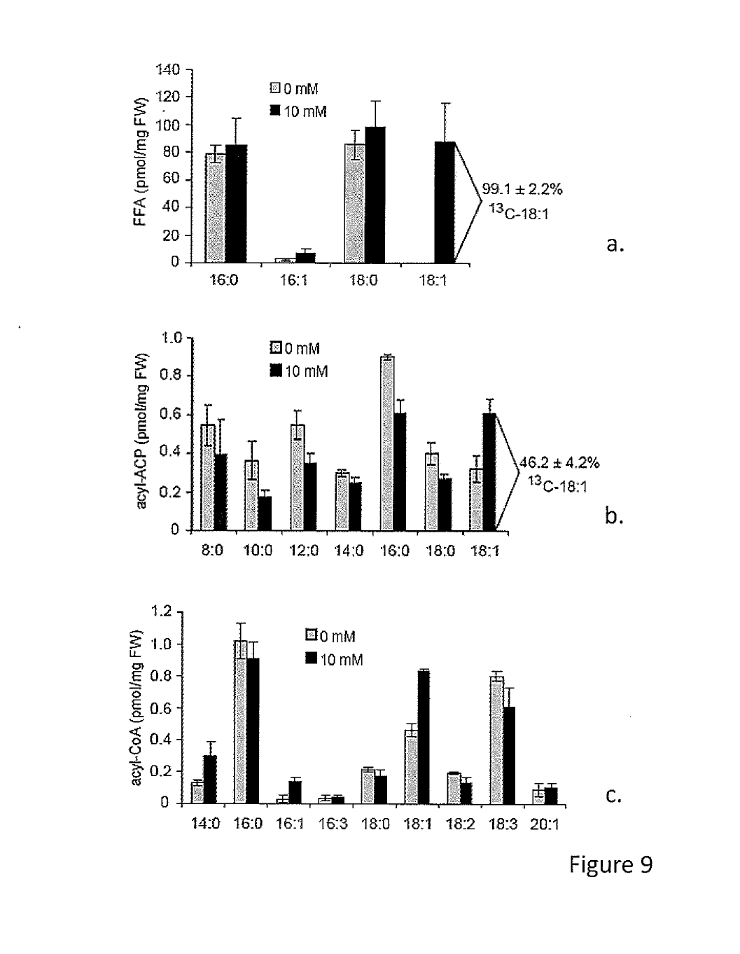

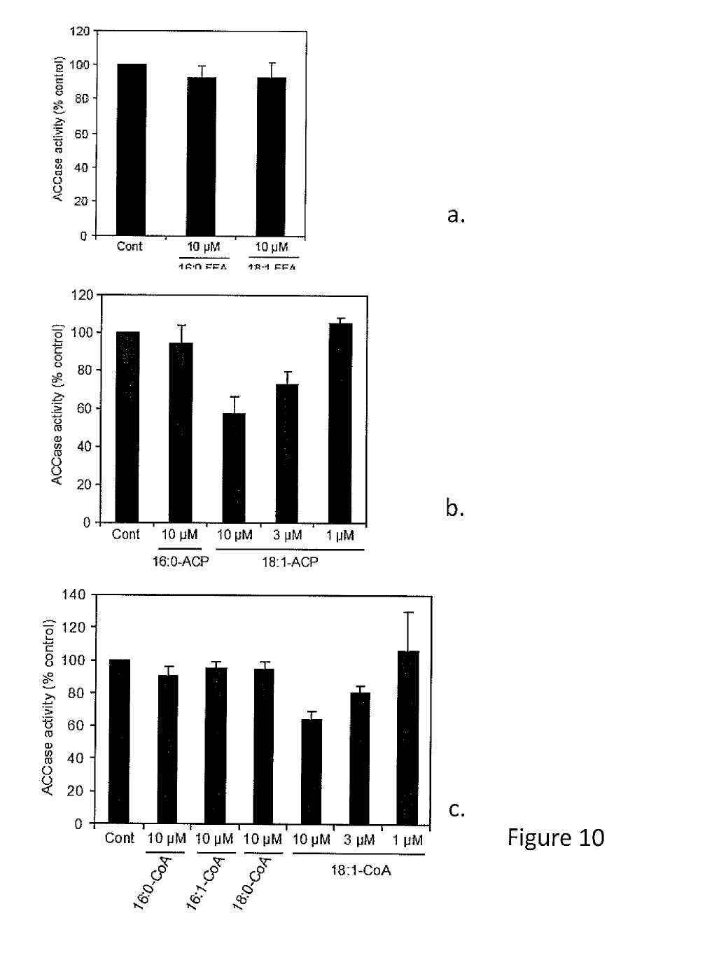

1986 Plant Physiol, 82, 771-779). Free fatty acids can be activated by esterification to CoA or ACP before being deposited in cellular lipids. Steady state pools acyl-ACP and acyl-CoA as well as free fatty acids (FFA) were measured in cells fed Tween-80. After three hours of feeding, 18:1 FFA appeared where there was none detected in untreated cells (FIG. 9a). Likewise, both 18:1-ACP and 18:1-CoA double in amount upon Tween-80 feeding while most other molecular species go down (FIG. 9b,c). In a separate experiment designed to distinguish the incorporation of fatty acids from Tween from secondary effects of feedback inhibition, FFA and acylACPs were analyzed in cells fed ¹³C-oleoyl-Tween. The results were that at 3 hours, 99.1±2.2% of 18:1 FFA and 46.2±4.2% of 18:1-ACP contained ¹³C-oleic acid (FIG. 9), meaning that the increases observed in these metabolites are the result of direct incorporation of fatty acids from Tween. 18:1 FFA, 18:1-ACP, and 18:1-CoA were tested for their effects on ACCase enzyme activity. First, the in vivo concentrations of these intermediates were calculated based on the range of values in FIG. 9 and the water content of the cells. The intracellular concentration of 18:1 FFA was estimated to be 0-100 μM. The estimated concentration of 18:1-ACP was 0.6-1.2 μM. However, considering that acyl-ACPs are restricted to the plastid, the volume of which in developing embryos was determined to be 10% of the cell (Mansfield and Briarty 1992, Can J Bot, 70, 151-164), the estimate increased to 5-10 μM. Total cellular 18:1-CoA was estimated to be 1-3 μM. These values established ranges of concentrations to test. When included in an assay of crude cell extract, up to 10 μM FFA had no effect on ACCase activity (FIG. 10a). Two considerations were made when testing the effects of acyl-ACPs and -CoAs. First, thioester bonds are labile in the basic conditions used in the ACCase assay, and combined with thioesterases present in crude extracts, acyl-ACPs and -CoAs could be degraded rapidly. Thioesterase activity was measured using the same conditions as the ACCase assay. With 3 μM (150 μmol assay⁻¹) 1-¹⁴C-oleoyl-ACP as substrate, thioesterase activity was found to be 7.9±2.2 pmol min⁻¹, meaning that by 20 min all of the acyl-ACP was degraded. Therefore, the duration of the ACCase assays was shortened from 30 to 10 min and metabolites were added immediately after the crude extract to lessen thioester bond cleavage. The second consideration was that E. coli ACCase was inhibited only by its own cognate acyl-ACPs (Davis and Cronan 2001, J Bacteriol, 183, 1499-1503). Therefore an ACP that was highly expressed in seeds (Safford et al. 1988 Eur J. Biochem., 174, 287-295) was cloned from the cell cultures and used in these experiments. Inhibition of ACCase was observed when 18:1-ACP was included at concentrations≧3 μM (FIG. 10b). A similar effect was observed with 18:1-CoA (FIG. 10c). In both cases, inhibition was partial. The specificity of inhibition to 18:1 acyl moieties is consistent with 18:1-containing Tweens causing maximum feedback (Table 2). As a control, Tween-80 itself was tested as an inhibitor of ACCase and was found to have no effect on enzyme activity (FIG. 7c), indicating that inhibition does not arise from the Tween-ester, rather that it arises from its downstream metabolites.

Previous studies on the feedback inhibition of fatty acid synthesis in plants demonstrated

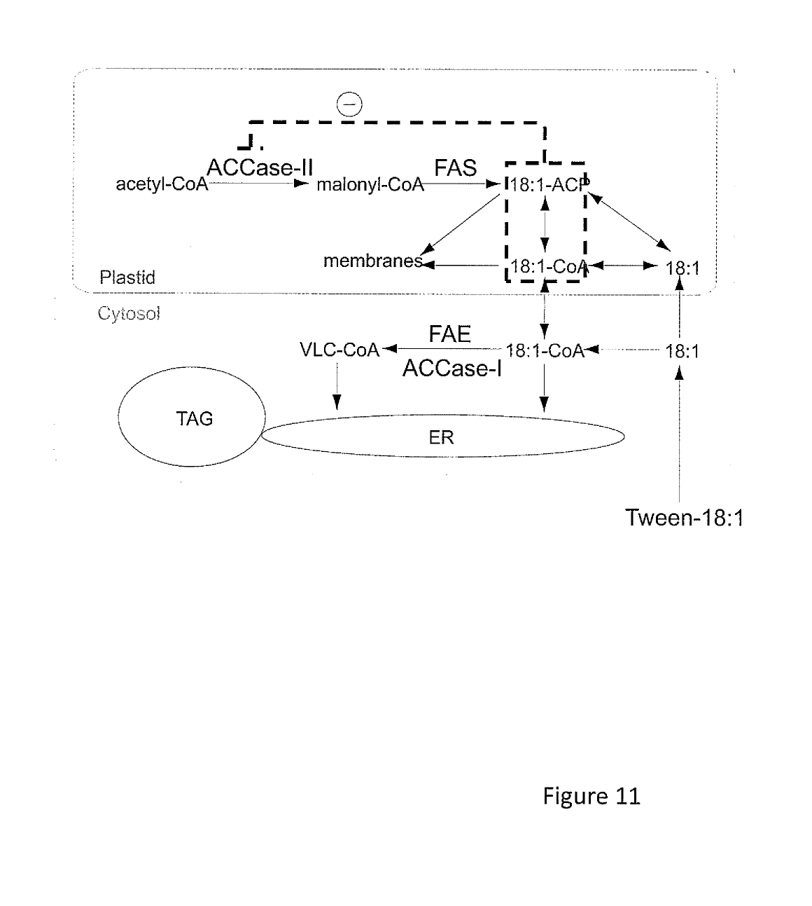

its existence in vegetative tissues with ACCase or FAS proposed as the site of inhibition (Ramli et al. 2002 Biochem J, 364, 393-401, Shintani and Ohlrogge 1995 Plant J, 7, 577-587, Terzaghi 1986a Plant Physiol, 82, 780-786). By using a unique embryo-like cell line, the existence of feedback regulation in a tissue where high rates of fatty acid synthesis are expected was demonstrated. Radiolabeling experiments were used to implicate plastidic ACCase as the specific site of inhibition. Transcriptional and posttranscriptional regulation of ACCase were discounted by analysis of gene expression and enzyme activity. Finally, feedback inhibition was correlated with increased amounts of 18:1-ACP and 18:1-CoA inhibiting ACCase activity in vitro. Based on these results, we propose a mechanism in which the concentrations of 18:1-ACP and/or 18:1-CoA mediate feedback regulation of plant fatty acid synthesis through their biochemical inhibition of plastidic ACCase (FIG. 11).

The B. napus cell line used here rapidly imported and incorporated fatty acids from Tweens. The cells were able to tolerate roughly 10-fold higher concentrations of Tween-80 and required higher levels to achieve equivalent feedback inhibition as previously reported for tobacco and soybean cell cultures (Shintani and Ohlrogge 1995 supra, Terzaghi 1986b, supra). That TAG is a strong sink for exogenous fatty acids in our B. napus cells reflects their propensity to synthesize storage oil. Such a metabolic predisposition could explain why higher concentrations of Tween-80 were needed to induce feedback as opposed to physical explanations such as the age or permeability of the cells. A previous study in tobacco reported the rate of production of intermediates of fatty acid synthesis, but not their actual pool size (Shintani and Ohlrogge 1995, supra). Reduced synthesis of long chain ACP led to the conclusion that they were unlikely to be involved in feedback. Indeed, in our system most acyl-ACPs decreased upon Tween feeding. However, 18:1-ACP actually increased as a result of incorporation of fatty acids from Tween. A plastid localized acyl-ACP synthetase has been identified capable of esterifying FFA to ACP (Koo et al. 2005, J Biol Chem, 279, 16101-16110). Acyl-ACP can also be synthesized from acyl-CoA and free ACP by a side reaction of KAS (Alberts et al. 1972, J Biol Chem, 247, 3190-3198) or by transfer of a fatty acid-phosphopantetheine arm from acyl-CoA to apo-ACP by holo-ACP synthase (Lambalot and Walsh 1995, J Biol Chem, 270, 24658-24661). How exogenous fatty acids enter the plastid is unknown. The amounts of acyl-ACP reported in this work are ˜10-fold higher than in spinach leaves, but the composition of individual molecular species is similar (Kopka et al. 1995 Anal Biochem, 224, 51-60). This quantitative difference may be attributable to the embryo-like identity (and associated higher rate of fatty acid synthesis) of the cells used. Reinforcing this notion is the fact that the acyl-CoA content is more like that from B. napus seeds than leaves (Larson and Graham 2001, Plant J, 25, 115-125).

The reduced rate of fatty acid synthesis during Tween feeding could result from metabolism of exogenous fatty acids leading to a shortage of free ACP and CoA. If this were the case, a shortage of either ACP or CoA would be manifest in reduced incorporation of both ¹⁴C-acetate and ¹⁴C-malonate into fatty acids. However, only the incorporation of ¹⁴C-acetate was reduced, indicating the effect was specific to ACCase activity. That plastidic ACCase is the target of feedback regulation is consistent with its role as the rate limiting step of fatty acid synthesis (Ohlrogge and Jaworski 1997, Annu Rev Plant Physiol Plant Mol Biol., 48, 109-136). Inhibition of F AS would predictably lead to an accumulation of malonyl-CoA. In the absence of F AS activity malonyl-CoA would be a dead-end product in the plastid, and therefore inhibition of ACCase is more efficient than that of F AS. In the cytosol, malonyl-CoA is required for flavonoid biosynthesis and loss of ACCase activity results in embryo lethality (Baud et al. 2003, Plant J, 33, 75-86). This side function of cytosolic ACCase may explain its evident immunity to the effects of feedback. Plastidic ACCase is known to be regulated by a variety of factors in vivo While apparently evident in other situations, transcriptional and post-translational regulation were not detected in the case of Tween-80 induced feedback. Light regulates ACCase indirectly through photosynthetically induced changes in stromal pH, Mg2+ concentration, and reduction potential. The cells used in this study were grown heterotrophically and in constant light, making it doubtful that photosynthesis had much influence on the stromal environment or ACCase activity. ATP is required for the ACCase reaction and its availability in the plastid could also influence activity. Long chain acyl-CoAs have been shown to reduce fatty acid synthesis in isolated plastids by inhibition of ATP import (Fox et al. 2001, Plant Physiol, 126, 1259-1265, Johnson et al. 2000, Biochem J, 348, 145-150). However, this was a general effect of all long chain CoAs and the combined amount of long chain acyl-CoAs was unaffected by Tween feeding making it unlikely that ATP import was inhibited. In addition, sterol biosynthesis and growth rate of the cells, both of which would be adversely affected by limited ATP supply, were also unaffected by Tween feeding.

Inhibition of ACCase by 18:1-ACP is consistent with the feedback mechanism of E. coli (Davis and Cronan 2001 J Bacteriol, 183, 1499-1503) and the prokaryotic evolutionary origin of plastidic ACCase (Cronan and Waldrop 2002 Prog Lipid Res, 41, 407-435). However, acyl-ACP was previously shown to not inhibit plant ACCase (Roesler et al. 1996, Plant Physiol, 113, 75-81). This discrepancy is likely due to several methodological differences. For one, E. coli ACCase was inhibited only when E. coli, but not spinach, acyl-ACPs were used (Davis and Cronan 2001, supra). In the current study, B. napus acyl-ACP was used to inhibit B. napus ACCase. Roesler and coworkers, on the other hand, used spinach acyl-ACP in assays with castor and pea ACCase. Therefore, it seems that inhibition of ACCase is dependent on the source of ACP.

Another difference is that in this and the E. coli studies, enzyme assays were conducted on crude extracts while Roesler and coworkers used semi-purified ACCases. The enzyme could have been modified (e.g. by phosphorylation or proteolysis) during purification to render it unresponsive to acyl-ACP, or there could be some other factor present in the crude extracts which facilitates inhibition of ACCase. Acyl-CoA inhibition of ACCase has been reported for yeast and animal ACCases (Ogiwara et al. 1978, Eur J Biochem, 89, 33-41) and for purified plant enzymes as well (Nikolau and Hawke 1984, Arch Biochem Biophys, 228, 86-96, Roessler 1990, Plant Physiol, 113, 75-81).

Supply of exogenous fatty acids in the form of Tween-80 is a non-physiological treatment that was used to elucidate a biochemical mechanism, raising the issue as to whether these results have implications for whole plant. Plastidic fatty acid synthesis terminates with the production of 16:0- or 18:1-ACP. These products are then cleaved by a thioesterase and the free fatty acids are converted to acyl-CoAs upon export from the plastid. ACCase (Thelen and Ohlrogge 2002, Arch Biochem Biophys, 400, 245-257), F AS (Roughan and Ohlrogge 1996, Plant Physiol, 110, 1239-1247), thioesterase (Shine et al. 1976, Arch Biochem Biophys, 172, 110-116), and acyl-CoA synthetase (Andrews and Keegstra 1983, Plant Physiol, 72, 735-740), are all associated with the chloroplast membrane and have been proposed to form a supercomplex that channels the intermediates of fatty acid synthesis from acetyl-CoA through acyl-CoA (Koo et al. 2004, Biol Chem, 279, 16101-16110, Thelen and Ohlrogge 2002, Arch Biochem Biophys, 400, 245-257). This membrane association is hypothesized to facilitate communication between the generation of fatty acids in the plastid and their demand in the cytosol. Within such a complex, the local concentrations of 18:1-ACP and 18:1-CoA could reach levels higher than the 1-3 μM range estimated above. ACCase was only partially inhibited by the physiological range of metabolite concentrations used here. Partial inhibition is sufficient though to account for the magnitude of feedback seen here. Inhibition of ACCase by 18:1-ACP is feasible, as acyl-ACP occurs primarily in the plastid. However, 18:1-CoA was previously undetectable in isolated chloroplasts (Post-Beittenmiller et al. 1991, J Biol Chem, 266, 1858-1865) and our results on acyl-CoA do not provide compartment specific information. However, there are enzymes in the plastid that can use 18:1-CoA as a substrate, such as G3P-acyltransferase (Frentzen et al. 1983Eur J Biochem, 129, 629-636). In addition, isolated chloroplasts are capable of incorporating exogenous 18:1-CoA into lipids, indicating the capacity for uptake and incorporation (Kjellberg et al. 2000, Biochim Biophys Acta, 1485, 100-110). A situation can be envisioned where cytosolic acylCoA is in low demand, causing diffusion of de novo 18:1-CoA to occur at a rate lower than its synthesis, thus leading to accumulation in the plastid. When combined with ability of KAS to transacyate free CoA with acyl-ACP (Alberts et al. 1972), it seems plausible that 18:1-CoA could accumulate in the plastid and inhibit ACCase.

That there was feedback at all in the cell line used here is interesting because it demonstrates that it can occur in seed-like tissues and when fatty acid synthesis is a primary metabolic function. This may explain why overexpression of ACCase results in very small increases in fatty acid production in seeds (Roesler et al. 1997, Plant Physiol, 113, 75-81). It also implies that oil seeds have evolved means of overcoming feedback inhibition. Thioesterases could be used to reduce the level of inhibitory 18:1-ACP, and indeed B. napus thioesterase prefers 18:1-ACP as substrate and is induced during embryo development (Hellyer et al 1992, Plant Mol Biol, 20, 763-780). Supporting this notion is the fact that feedback inhibition is relieved in E. coli by overexpression of a thioesterase (Jiang and Cronan 1994, J Bacterial, 176, 2814-2821) and in B. napus indirect reduction in 18:1-ACP by overexpression of a medium chain thioesterase resulted in higher rates of fatty acid synthesis (Eccleston and Ohlrogge 1998, Plant Cell, 10, 613-622). Feedback inhibition may also explain the shared control of oil accumulation between synthesis and assembly in B. napus (Ramli et al. 2002a, Biochem J, 364, 393-401). Diacylglycerol acyltransferase (DGAT), which consumes acyl-CoA in the cytosol, was suggested to exert control over oil accumulation (Perry et al. 1999 Phytochemistry, 52, 799-804, Weselake et al. 2008 Prog Lipid Res, 38, 401-460) and overexpression of this enzyme in Arabidopsis results in enhanced oil content (Jako et al. 2001, Plant Physiol, 126, 861-874). Increased fatty acid synthesis is a logical prerequisite for elevated oil content. Conversely, the Arabidopsis asi1(tag1) mutant deficient in DGAT has less oil than wild type and reduced incorporation of ¹⁴C-acetate into lipids, indicating reduce fatty acid synthesis (Katavic et al. 1995, Plant Physiol, 108, 399-409). Premised on these results and the current study, DGAT might exert control over oil accumulation by consuming acyl-CoA in the cytosol, thus driving vectorial export of de nova fatty acids from the plastid and preventing feedback inhibition.

Fatty acid biosynthesis is an essential biosynthetic pathway with high demand for ATP and reductants. It is therefore seems logical that its regulation would occur at many levels. This work was designed to address early events in biochemical feedback. However, feedback is persistent during prolonged Tween feeding (this study, Shintani and Ohlrogge 1995) and by analogy with other systems may involve a dynamic series of mechanisms capable of rapid and then persistent response to oversupply of fatty acids. In addition, the fact that inhibition of, ACCase by acyl-ACP has only been observed when assaying crude cell extracts leaves open the possibility that some other factor is required for inhibition.

### Example 7

### Construction of T-DNA Vectors and Isolation of Transgenic Plants with Alternative ACC Subunits

Using standard recombinant DNA techniques the following chimeric genes are created by operably linking the following DNA fragments:

### Vector MS1

a double enhanced CaMV35S promoter region
a DNA region encoding a chloroplast targeting signal (SEQ ID 36-37)
the DNA region of SEQ ID No. 1 from nucleotide position 331 to nucleotide position 1860 encoding biotin carboxylase from Metallosphaera sedula
a transcription termination and polyadenylation signal from 3′ nopalinesynthase gene.

### Vector MS2

a double enhanced CaMV35S promoter region
a DNA region encoding a chloroplast targeting signal (SEQ ID 36-37)
the DNA region of SEQ ID No. 1 from nucleotide position 1860 to nucleotide position 2360 encoding biotin carboxylase carrier protein from Metallosphaera sedula
a transcription termination and polyadenylation signal from 3′ nopalinesynthase gene.

### Vector MS3

a double enhanced CaMV35S promoter region
a DNA region encoding a chloroplast targeting signal
the DNA region of SEQ ID No. 2 from nucleotide position 659 to nucleotide position 2230 encoding carboxytransferase protein from Metallosphaera sedula
a transcription termination and polyadenylation signal from 3′ nopalinesynthase gene.

### Vector CS1

a double enhanced CaMV35S promoter region
a DNA region encoding a chloroplast targeting signal (SEQ ID 36-37)
the DNA region of SEQ ID No. 6 encoding biotin carboxylase carrier protein from Cenarchum symbiosum
a transcription termination and polyadenylation signal from 3′ nopalinesynthase gene.

### Vector CS2

a double enhanced CaMV35S promoter region
a DNA region encoding a chloroplast targeting signal (SEQ ID 36-37)
the DNA region of SEQ ID No. 8 encoding biotin carboxylase from Cenarchum symbiosum
a transcription termination and polyadenylation signal from 3′ nopalinesynthase gene.

### Vector CS3

a double enhanced CaMV35S promoter region
a DNA region encoding a chloroplast targeting signal (SEQ ID 36-37)
the DNA region of SEQ ID No. 10 encoding carboxytransferase from Cenarchum symbiosum
a transcription termination and polyadenylation signal from 3′ nopalinesynthase gene.

### Vector CA1

a double enhanced CaMV35S promoter region
a DNA region encoding a chloroplast targeting signal (SEQ ID 36-37)
the DNA region of SEQ ID No. 12 encoding biotin carboxylase from Chloroflexus aurantiacus
a transcription termination and polyadenylation signal from 3′ nopalinesynthase gene.

### Vector CA2

a double enhanced CaMV35S promoter region
a DNA region encoding a chloroplast targeting signal (SEQ ID 36-37)
the DNA region of SEQ ID No. 14 encoding biotin carboxylase carrier protein from Chloroflexus aurantiacus
a transcription termination and polyadenylation signal from 3′ nopalinesynthase gene.

### Vector CA3

a double enhanced CaMV35S promoter region
a DNA region encoding a chloroplast targeting signal (SEQ ID 36-37)
the DNA region of SEQ ID No. 16 encoding biotin carboxytransferase alpha from Chloroflexus aurantiacus
a transcription termination and polyadenylation signal from 3′ nopalinesynthase gene.

### Vector CA4

a double enhanced CaMV35S promoter region
a DNA region encoding a chloroplast targeting signal (SEQ ID 36-37)
the DNA region of SEQ ID No. 18 encoding biotin carboxytransferase beta from Chloroflexus aurantiacus
a transcription termination and polyadenylation signal from 3′ nopalinesynthase gene.

The chimeric genes of vectors MS1, MS2 and MS3 are combined in one T-DNA vector, further comprising a selectable marker gene.

Likewise, the chimeric genes of vectors CS1, CS2 and CS3 are combined in one T-DNA vector, further comprising a selectable marker gene.

Also, the chimeric genes of vectors CA1, CA2, CA3 and CA4 are combined in one T-DNA vector, further comprising a selectable marker gene.

The T-DNA vectors are introduced into Agrobacterium strains comprising a helper Ti-plasmid using conventional methods. Hypocotyl explants of Brassica napus are obtained, cultured and transformed essentially as described by De Block et al. (1989), Plant Physiol. 91: 694) to transfer the chimeric genes into Brassica napus plants.

Transgenic Brassica napus plant are identified and analyzed for increased oil content.

### Example 8

### Construction of T-DNA Vectors and Isolation of Transgenic Plants Overexpressing FAT a Protein

Using standard recombinant DNA techniques the following chimeric gene is created by operably linking the following DNA fragments:

### Vector RC1

a double enhanced CaMV35S promoter region
the DNA region of SEQ ID No. 20 encoding a FAT protein from Ricinus communis
a transcription termination and polyadenylation signal from 3′ nopalinesynthase gene.

The chimeric gene is introduced between left and right T-DNA borders together with a selectable marker gene.

The T-DNA vector is introduced into an Agrobacterium strain comprising a helper Ti-plasmid using conventional methods. Hypocotyl explants of Brassica napus are obtained, cultured and transformed essentially as described by De Block et al. (1989), Plant Physiol. 91: 694) to transfer the chimeric gene into Brassica napus plants.

Transgenic Brassica napus plant are identified and analyzed for increased oil content.

### Example 9

### Construction of T-DNA Vectors and Isolation of Transgenic Plants Overexpressing Acyl-CoA Binding Proteins

Using standard recombinant DNA techniques the following chimeric gene is created by operably linking the following DNA fragments:

### Vector ACBP4

a double enhanced CaMV35S promoter region
the DNA region of SEQ ID No. 22 from nucleotide 103 to nucleotide 2109 encoding a ACBP4 protein from Arabidopsis thaliana
a transcription termination and polyadenylation signal from 3′ nopalinesynthase gene.

### Vector ACBP6

a double enhanced CaMV35S promoter region
the DNA region of SEQ ID No. 24 from nucleotide 106 to nucleotide 384 encoding a ACBP6 protein from Arabidopsis thaliana
a transcription termination and polyadenylation signal from 3′ nopalinesynthase gene.

The chimeric genes are introduced (separately) between left and right T-DNA borders together with a selectable marker gene.

The T-DNA vector is introduced into an Agrobacterium strain comprising a helper Ti-plasmid using conventional methods. Hypocotyl explants of Brassica napus are obtained, cultured and transformed essentially as described by De Block et al. (1989), Plant Physiol. 91: 694) to transfer the chimeric genes into Brassica napus plants.

Trangenic Brassica napus plant are identified and analyzed for increased oil content.

### Example 10

### Construction of T-DNA Vectors and Isolation of Transgenic Plants overexpressing Acyl-CoA binding proteins

CsACCase (ACCase from Cenarchaeum symbiosum) subunits each equipped with chloroplast transit peptide from Ricinus communis steearoyl-ACP desaturase were cloned into the pSAT expression system as described in Tzfira et al., 2005 Plant Mol. Biol. 57, 503-516. For codon optimized CsACCase subunits, BCCP (SEQ ID No: 129-130) was cloned into pSAT1-mcs with EcoRI and BamHI, BC (SEQ ID Nos: 131-132) into pSAT4-mcs with BglII and XbaI, and CT (SEQ ID Nos: 133-134) into pSAT5-mcs using EcoRI and BamHI. All three expression cassettes contained the ³⁵S promoter.

pPZP-RCS2-nptII-dsRed containing the expression cassettes from pSAT4-nptII (Genbank accession number AY818371) and pSAT6-DsRed2-C1 (Genbank accession number AY818375) was used for cloning CsACCase expression cassettes and also as empty vector control. pPZP-RCS2 was designed for cloning multiple expression cassettes (Goderis et al., 2002) and is based on the binary vector pPZP200 (genbank accession U10460, Hajdukiewicz et al., 1994).

Expression cassettes containing optimized CsACCase genes were excised from respective pSAT vectors and were inserted into pPZP-RCS2-nptII-dsRed. Because nptII was in the pSAT4 insertion site of pPZP-RCS2, the final construct containing CsACCase genes does not contain nptII. It was replaced with the CsBC gene which was cloned into pSAT4-mcs and therefore had to be inserted in the pSAT4 insertion site of pPZP-RCS2.

The final construct has the pPZP-RCS2 backbone with CsBCCP in site 1, CsBC in site 4, CsCT in site 5, and DsRed in site 6. All cassettes are driven by the ³⁵S promoter.

The T-DNA vectors (with or without CsACCase subunits) were introduced into an Agrobacterium strain comprising a helper Ti-plasmid using conventional methods, and the Agrobacterium strain was used to transform Arabidopsis in a conventional manner.

Transgenic Arabidopsis lines which were either transformed with the CsACCase subunits (ACCases L1-13) or with the “empty vector” (EVL1-9) were obtained. T2 seeds were analysed for their seed oil content (3 samples per line) by determining the content of fatty acid methyl ester (FAME) per seed (expressed in μg).

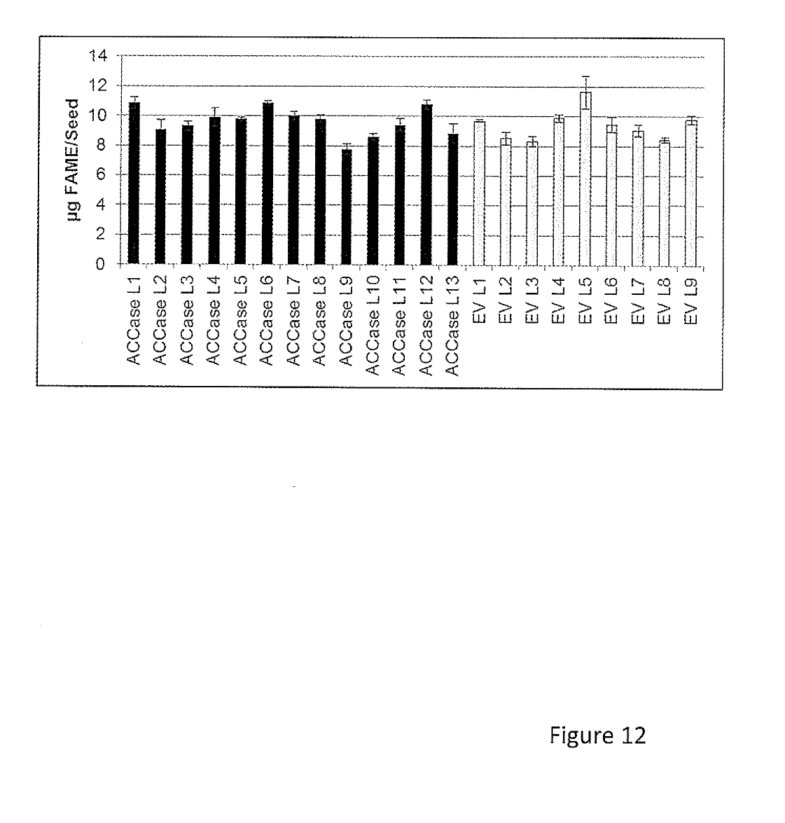

The results are summarized in Table 3 and graphically represented in FIG. 12. As can be deduced from these results, several Accase Lines have a seed oil content which is higher than several of the EV L control lines.

TABLE 3
Seed oil content

| | Oil content | Standard |
|---|---|---|
| | (μg FAME/seed) | Deviation |
| ACCase L1 | 10.85384 | 0.388431 |
| ACCase L2 | 9.077156 | 0.652935 |
| ACCase L3 | 9.310987 | 0.316129 |
| ACCase L4 | 9.891197 | 0.609785 |
| ACCase L5 | 9.775171 | 0.143841 |
| ACCase L6 | 10.86957 | 0.157221 |
| ACCase L7 | 10.02674 | 0.283685 |
| ACCase L8 | 9.807127 | 0.241436 |
| ACCase L9 | 7.767996 | 0.350664 |
| ACCase L10 | 8.608321 | 0.20921 |
| ACCase L11 | 9.416196 | 0.443759 |
| ACCase L12 | 10.77586 | 0.302835 |
| ACCase L13 | 8.839128 | 0.661011 |
| EV L1 | 9.696186 | 0.096824 |
| EV L2 | 8.51547 | 0.43468 |
| EV L3 | 8.294166 | 0.336582 |
| EV L4 | 9.871668 | 0.238142 |
| EV L5 | 11.62791 | 1.081375 |
| EV L6 | 9.430997 | 0.49808 |
| EV L7 | 9.038867 | 0.397765 |
| EV L8 | 8.429334 | 0.178853 |
| EV L9 | 9.797518 | 0.271336 |

1. A method to increase oil content in cells of a plant, comprising the step of preventing feedback inhibition by 18:1-Coenzyme A or 18:1-Acyl Carrier Protein of the plastidic acetyl CoA-carboxylase enzyme in said cells of said plant by expressing in said plant cells an acetyl CoA-carboxylase enzyme or subunit thereof from an organism that uses the 3-hydroxypropionate cycle for carbon fixation.

2-5. (canceled)

6. The method according to claim 1, wherein said organism is Sulfolobales, Cenarchaeles, Archeaoglobales, Desulfurococcales, Thermoproteales, Thermococcales or Halobacterales.

7. The method according to claim 1, wherein said organism is Metallosphaera sedula, Acidianus brierkyi, Sulfolobus solfataricus, Sulfolobus tokodaii, Sulfolobus acidocaldaricus, Cenarcheum symbiosum, Archaeoglobus fulgidus, Hyperthermus butylicus, Staphylotthermus marinus, Thermofilum pendens, Ingicoccus hospitalis, Pyrobaculum aerophilum, Pyrobaculum islandicum, Pyrobaculum calidifontis, Pyrobaculum furisous, Pyrobaculum abyssi, Pyrobaculum horykoshii, Haloarcula marismortui, Halobacterium sp. NRC-1, Haloquatratum walsbyi, Halorubrum lacusprofundi or Natromonas pharaonis.

8. The method according to claim 1, wherein said organism is Metallosphaera sedula, Acidianus brierleyi or Cenarcheum symbiosum.

9. (canceled)

10. The method according to 1, wherein said acetyl CoA-carboxylase enzyme or subunit thereof is any of SEQ ID Nos. 46-128.

11. The method according to claim 1, wherein said plant cell comprises a DNA molecule comprising the following operably linked DNA fragments:
a) a plant expressible promoter;
b) one or more coding regions encoding one or more acetyl CoA-carboxylase subunits having an amino acid sequence of SEQ ID Nos. 2, 3, 5, 7, 9, 11, 13, 15, 17 or 19; or a coding region having 70% sequence identity with an amino acid sequence of SEQ ID Nos. 2, 3, 5, 7, 9, 11, 13, 15, 17 or 19, and having acetyl CoA carboxylase enzymatic activity;
and optionally
c) a transcription termination and/or polyadenylation region functional in plant cells.

12. The method of claim 11, wherein said DNA molecule further comprises a DNA region encoding a chloroplast targeting peptide.

13. The method according to claim 11, wherein said coding region comprises the nucleotide sequence of SEQ ID No. 1 from the nucleotide at position 331 to the nucleotide at position 1860, SEQ ID No. 1 from the nucleotide at position 1860 to the nucleotide at position 2360, SEQ ID No. 4, SEQ ID No. 6, SEQ ID No. 8, SEQ ID No. 10, SEQ ID No. 12, SEQ ID No. 14, SEQ ID No. 16 or SEQ ID No. 18 or a coding region having 70% nucleotide sequence identity therewith.

14. The method of claim 11, wherein said plant expressible promoter is a promoter which is expressed in plastids and wherein said termination and/or polyadenylation region is a transcription termination region functional in plastids.

15-26. (canceled)

27. The method according to claim 1, wherein said plant cell is in a plant.

28. The method according to claim 1, wherein said oil content is increased in oil storage parts of said plant.

29. The method according to claim 28, wherein said oil content is increased in seeds of said plant.

30. The method according to claim 27, wherein said plant is an oleipherous plant selected from Brassica oilseeds, sunflower, safflower, soybean, palm, Jatropha, flax, crambe, camelina, corn, sesame, or castor beans.

31. The method according to claim 30, wherein said plant is Brassica napus, Brassica campestris (raga), Brassica juncea, or Brassica carinata.

32. A plant comprising in its plastids one or more ACCase variant enzymes or subunits thereof which are less sensitive to feedback inhibition by 18:1-Coenzyme A or 18:1-Acyl Carrier Protein than a wild-type acetyl CoA-carboxylase of said plant, wherein said acetyl CoA-carboxylase enzyme or subunit thereof is from an organism that uses the 3-hydroxypropionate cycle for carbon fixation.

33. (canceled)

34. The plant according to claim 32, wherein said organism is Sulfolobales, Cenarchaeles, Archeaoglobales, Desulfurococcales, Thermoproteales, Thermococcales or Halobacterales.

35. The plant according to claim 34, wherein said organism is Metallosphaera sedula, Acidianus brierleyi, Sulfolobus solfataricus, Sulfolobus tokodaii, Sulfolobus acidocaldaricus, Cenarcheum symbiosum, Archaeoglobus fulgidus, Hyperthermus butylicus, Staphylotthermus marinus, Thermofilum pendens, Ingicoccus hospitalis, Pyrobaculum aerophilum, Pyrobaculum islandicum, Pyrobaculum calidifontis, Pyrobaculum furisous, Pyrobaculum abyssi, Pyrobaculum horykoshii, Haloarcula marismortui, Halobacterium sp. NRC-1, Haloquatratum walsbyi, Halorubrum lacusprofundi or Natromonas pharaonis.

36. The plant according to claim 32, wherein said organism is Metallosphaera sedula, Acidianus brierleyi or Cenarcheum symbiosum.

37. The plant according to claim 32, wherein said acetyl CoA-carboxylase enzyme or subunit thereof is from Chloroflexus auranticus.

38. The plant according to claim 32, comprising a DNA molecule comprising the following operably linked DNA fragments:
a) a plant expressible promoter;
b) one or more coding regions encoding one or more acetyl CoA-carboxylase subunits having an amino acid sequence of SEQ ID Nos. 2, 3, 5, 7, 9, 11, 13, 15, 17 or 19; or a coding region having 70% sequence identity with an amino acid of SEQ ID Nos. 2, 3, 5, 7, 9, 11, 13, 15, 17 or 19 or any of SEQ ID Nos. 46-128 and having acetyl CoA carboxylase enzymatic activity;
and optionally
c) a transcription termination and/or polyadenylation region functional in plant cells.

39. The plant according to claim 38, wherein said DNA molecule further comprises a DNA region encoding a chloroplast targeting peptide.

40. The plant according to claim 38, wherein said coding region comprises the nucleotide sequence of SEQ ID No. 1 from the nucleotide at position 331 to the nucleotide at position 1860, SEQ ID No. 1 from the nucleotide at position 1860 to the nucleotide at position 2360, SEQ ID No. 4, SEQ ID No. 6, SEQ ID No. 8, SEQ ID No. 10, SEQ ID No. 12, SEQ ID No. 14, SEQ ID No. 16 or SEQ ID No. 18 or a coding region having 70% nucleotide sequence identity therewith.

41. The plant according to claim 38, wherein said plant expressible promoter is a promoter which is expressed in plastids and wherein said termination and/or polyadenylation region is a transcription termination region functional in plastids.

42. A cell, tissue, oil storage tissue or seed of a plant according to claim 32.

43. (canceled)

44. A chimeric DNA comprising the following operably linked DNA fragments
a) a plant expressible promoter;
b) one or more coding regions encoding one or more acetyl CoA-carboxylase subunits having an amino acid sequence of SEQ ID Nos. 2, 3, 5, 7, 9, 11, 13, 15, 17 or 19; or a coding region having 70% sequence identity with an amino acid sequence of SEQ ID Nos. 2, 3, 5, 7, 9, 11, 13, 15, 17 or 19 or any of SEQ ID Nos. 46-128 and having acetyl CoA carboxylase enzymatic activity;
and optionally
c) a transcription termination and/or polyadenylation region functional in plant cells.

45-49. (canceled)

50. A method of producing food, feed, or an industrial product comprising
a) obtaining the plant or a part thereof, of claim 32; and
b) preparing the food, feed or industrial product from the plant or part thereof.

51. The method of claim 50 wherein
a) the food or feed is oil, meal, grain, starch, flour or protein; or
b) the industrial product is biofuel, fiber, industrial chemicals, a pharmaceutical or a nutraceutical.

52. The method according to claim 11, wherein the coding region has 95% sequence identity with an amino acid sequence of SEQ ID Nos. 2, 3, 5, 7, 9, 11, 13, 15, 17 or 19.

53. The method according to claim 13, wherein the coding region has 95% nucleotide sequence identity therewith.

54. The method according to claim 38, wherein the coding region has 95% sequence identity with an amino acid of SEQ ID Nos. 2, 3, 5, 7, 9, 11, 13, 15, 17 or 19 or any of SEQ ID Nos. 46-128.

55. The method according to claim 40, wherein the coding region has 95% nucleotide sequence identity therewith.

56. The method according to claim 44, wherein the a coding region has 95% sequence identity with an amino acid of SEQ ID Nos. 2, 3, 5, 7, 9, 11, 13, 15, 17 or 19 or any of SEQ ID Nos. 46-128.

57. The method according to claim 1, wherein the expression is in the plastids of the plant cell.


SEQUENCE LISTING (134 sequences)

SEQ ID NO: 1 - DNA, 3152 bp, Metallosphaera sedula
  CDS (331)..(1860)
  CDS (1863)..(2360)
gatcggcacagaaggtaatcttctcgacttgtaggaggcttaaaattggagatggtgtaagggtagaaagctataagagggacaggtttatagaaatttatctagtaagtaacgataaatataagataatagaaaacggttatatcaggagggaactaactatatcgggacaggaattaaaggactttctaaaggggatactttcgatcgagtttcccaggagtaacgtgctttacctgagcgaagtacatagtataatataaaacttcataagaaaaactgatagataaaaactttttaaactattcgagttatatttatgtgattcttatgccaccctttagtagagttttggttgcaaacaggggagaaattgcagtaagggtaatgaaggcaataaaggaaatgggaatgacagcaatagctgtttactctgaggctgacaagtacgcagtccacgttaagtatgccgatgaagcttattatattggaccctcgccggccttggaaagttacctcaacataccccacatcattgacgcagcggagaaggctcacgctgacgctgttcatcctggatatggattcttgtcggagaatgctgacttcgtggaggcagttgaaaaggcaggaatgacttacataggtccctctgctgaggtcatgagaaagataaaggataagctggatgggaaaaggatagcccagttatctggtgtccccattgcccctggctcggatggccccgtagaatccattgacgaggctcttaagttggctgagaagataggataccccatcatggttaaggccgctagcgggggtggtggagtaggtataacaaagatagatacacctgaccagctcattgacgcatgggaaagaaacaagaggttagctacacaagccttcggacgatctgatctatacatagaaaaagccgccgtaaaccctaggcacattgagtttcagttaattggcgataagtacggcaactatgtcgttgcttgggagagggaatgtactattcagagaagaaaccagaagttgatagaggaggcaccatctccagcaatcacaatggaagaaaggtcacgaatgttcgagcctatatacaaatatgggaagttaattaattactttaccctgggtactttcgagacagttttctctgatgccacaagggagttctacttccttgagctgaacaaaaggcttcaggtagaacacccagttactgagttaatattcagaattgatctggtaaagctacagataaggctagctgcaggagaacatttgccattcacgcaggaggaactcaacaagagggcgagaggtgcagcaatagagttcaggataaatgccgaggatccaataaataatttcagcggaagctcaggtttcattacgtactacagggagcccacgggtcctggagtgagaatggatagcggtgtaacggagggaagctgggtacctcctttctacgactctctagtatcgaagttgattgtgtatggagaagacaggcaatacgcaatacaaactgccatgagggcactagacgattacaagattggcggagtcaaaacgactataccgctatacaagctcatcatgagggatcccgactttcaggaaggaaggttcagtactgcctatatttcccagaagattgactcaatggttaagaaactgaaggccgaagaggagatgatggcttcagtggccgcagttcttcagagcaggggactccttagaaagaaggcttcagctcctcaggagcaggcgaaaccaggctcaggatggaagagttacggtatcatgatgcagagcactcctagggtgatgtggggatgaaactgtatagggttcatgcggatacaggagataccttcattgtggcccacgatcaaaaggaaaacaaggacagactaaagacggaaaataacgagtttgagatagagtatgtcggtcagggtacaagggaaggagaaataatcctgaagattaacggtgagatgcacagggtcttcatagacaacggatggataattcttgacaatgcaaggatattcagggcagagagagttacagagcttcccactcaggaaggacagacactggacgagatgatcaaaggtaaggagggagaagtgctatcaccgcttcagggcagagtagttcaggtcagggttaaggaaggcgatgcggtgaataagggacagcccttgctatcgattgaggccatgaaatctgagaccatagtgtcggcaccaataagcgggctagtggagaaggtattagttaaggcaggtcaaggagtaaagaagggagatatcctagtggtgataaagtaagctggttattctgggaggcttatggaatttcttttaagaatatttcttatatgttttgaaagttcttttgagtaaccccttcttccaccctccgcaattattagttttgccttggagttgtccacgtaatatattaaatcctcaaaatctgcatgatcactaaatgctatactggtggataccttgtctatcctcttaacgggaacctcaaattcccatccggaaagtagaaagtttgcatgaagagagtccctttgcttgaattgattaaagtggagaaactctatgtaccatcggtccttcaatatgccctgggcctcggcttcctccttggagaagacgtcctcgatttgtatcccgttggagatggcgatccgcgttatatcccgtacctttccgtcaactataaatggggcaatgacaccgttcttcctaagcaccctcattacctcctgtaactttccgtgatagccgtatattcttacgggcattttcactaccgcgtcatttatgaaatccgacatcagctgttcaacctctcccttaaactttctcgtaaattccggtttgccgtaagtggactctataattagtatgtcagggttaagtatgggggtccccttttccggattttgaatcccctgtataggctatggtttcctcgttggtgataatttctacttgcgccgatccaaacacgtgatcggaggggtgaagagttagtctctcttcatccacgttgatcgtaaccccgtaattcaaatctagtctcttatttcgggcaatcta

SEQ ID NO: 2 - protein, 510 aa, Metallosphaera sedula
MPPFSRVLVANRGEIAVRVMKAIKEMGMTAIAVYSEADKYAVHVKYADEAYYIGPSPALESYLNIPHIIDAAEKAHADAVHPGYGFLSENADFVEAVEKAGMTYIGPSAEVMRKIKDKLDGKRIAQLSGVPIAPGSDGPVESIDEALKLAEKIGYPIMVKAASGGGGVGITKIDTPDQLIDAWERNKRLATQAFGRSDLYIEKAAVNPRHIEFQLIGDKYGNYVVAWERECTIQRRNQKLIEEAPSPAITMEERSRMFEPIYKYGKLINYFTLGTFETVFSDATREFYFLELNKRLQVEHPVTELIFRIDLVKLQIRLAAGEHLPFTQEELNKRARGAAIEFRINAEDPINNFSGSSGFITYYREPTGPGVRMDSGVTEGSWVPPFYDSLVSKLIVYGEDRQYAIQTAMRALDDYKIGGVKTTIPLYKLIMRDPDFQEGRFSTAYISQKIDSMVKKLKAEEEMMASVAAVLQSRGLLRKKASAPQEQAKPGSGWKSYGIMMQSTPRVMWG

SEQ ID NO: 3 - protein, 166 aa, Metallosphaera sedula
KLYRVHADTGDTFIVAHDQKENKDRLKTENNEFEIEYVGQGTREGEIILKINGEMHRVFIDNGWIILDNARIFRAERVTELPTQEGQTLDEMIKGKEGEVLSPLQGRVVQVRVKEGDAVNKGQPLLSIEAMKSETIVSAPISGLVEKVLVKAGQGVKKGDILVVIK

SEQ ID NO: 4 - DNA, 2547 bp, Metallosphaera sedula
  CDS (659)..(2230)
acgtcctcctgctgggcttcatctccaccttctattacgtcttttatagttactggatggggataaagaacatgcccaaggagtactgggaagtgatggataacttgaacttaaccttttggcaaaggttgagaagggtggtaatcccctcagccatgccatacatagtggcagggttaacgagtacggtgaacagtgcatggggaggcctggccataggagagtactggccagatatatatgacgggagaaccctcgaggtacatcagggactaatgagggagctggcactagcagatagtcagggcaaacttgctctagtgggttggctttcaatccttttcgccattgttgtggttatatactccctcttcttcactaggaagctcatggatctagctagacagaaatacgtggctgaggaagggatatacgctgcctaacccttcatgccaaggctcacacttttaacgctactttacatcttgttctcaccagtgatgaggaatccgggatccgatctgtgagggcccatacgcgaactctgagggtgtagtaaaaagtccgttataattataaaatatcagtatgtcaatattaactaaagtttaaatgataaaaattttaagagagaacttacaataggtcttgattagacatgactgcaacttttgaaaaaccggatatgtcaaaactagttgaggaattgagagccctaaaggccaaggcttacatgggtggaggagaggagagagtacaggctcaacatgctaagggcaagctgacagcgagggagaggttaaatctcctattcgatgaggggacctttaacgaggtcatgacctttgccacgacaaaggctactgagtttggattggataaaagcaaggtctacggagacggcgtagtaactggatggggacaggtcgagggaaggactgtatttgcatttgcccaggacttcacgtctataggaggcacgctaggggagactcacgcgtctaaaatagctaaggtttatgagctcgcattaaaggttggagcacctgtagttgggataaatgattctggaggagccagaatacaagagggcgcagtcgcactagagggttacggtacggtctttaaggccaacgtgatggcatctggcgtagttccccagataaccataatggctggtccagctgcgggaggtgcagtttactcaccagccctcactgacttcataatcatgataaagggcgacgcctactacatgtttgtgaccggtccagagatcacaaaggtggtgttgggtgaggacgtttcattccaagacctaggtggagccgtaattcacgcaactaaatcaggcgtggttcacttcattgcggagaacgaacaagattccattaacataaccaagaggttgctctcttacctaccctctaacaacatggaggagccacccttcatggacacgggagaccctgcggacagggaaatgaaggacgtggagagcgttgttccaactgacaccgtcaagccctttgatatgagagaggtaatatacaggactgtggacaacggcgagttcatggaggtgcagaagcattgggctcagaacatggtggttggatttggaagggtagccgggaacgtggtaggtatagtagcaaataactccgcccatctgggggcagccatagatatagacgcctcagacaaggcggccaggttcataaggttctgtgacgcttttaatattcccttgattagcttggtggacactcctggttacatgccgggaacagaccaggaatataagggcatcattaggcacggagcaaagatgttgtacgcctttgctgaggcaacagttcccaaggtcactgtggtggttagaaggtcctacggtggcgctcacatcgccatgagcataaagagccttggagcggatctcatatatgcttggccctctgcagagatagcggtgactgggcctgagggggccgtgaggatcctgtacaggagagaaattcagaacagcaagtctcctgacgatctcatcaaggagagaatagctgagtacaagaagttgttcgccaacccctattgggcagctgagaagggattgattgacgacgtaatagagcccaaggatacgaggaaggtaatagcgtcagccttgaagatgttaaagaacaagagggagttcaggtaccccaagaagcatggaaatatacccctctaaggccttcttttctttaccaagagcctatttccctcaattctagtttcttgggctaagacccttaacaccagctccagctttttcagggtatcctgatctccctcaaacacaagttcctctccgtccttagccctaaagaactcctccaataactcttgcagcgtccttgacaaccttctcacctaccttctctcctatgagtgccttaactttctccgggttcatgactaggtcctccggcttagttaagccgttctgatacagtaaccttcccctcttccttccaattccaggaactctcactaggtctagca

SEQ ID NO: 5 - protein, 524 aa, Metallosphaera sedula
MTATFEKPDMSKLVEELRALKAKAYMGGGEERVQAQHAKGKLTARERLNLLFDEGTFNEVMTFATTKATEFGLDKSKVYGDGVVTGWGQVEGRTVFAFAQDFTSIGGTLGETHASKIAKVYELALKVGAPVVGINDSGGARIQEGAVALEGYGTVFKANVMASGVVPQITIMAGPAAGGAVYSPALTDFIIMIKGDAYYMFVTGPEITKVVLGEDVSFQDLGGAVIHATKSGVVHFIAENEQDSINITKRLLSYLPSNNMEEPPFMDTGDPADREMKDVESVVPTDTVKPFDMREVIYRTVDNGEFMEVQKHWAQNMVVGFGRVAGNVVGIVANNSAHLGAAIDIDASDKAARFIRFCDAFNIPLISLVDTPGYMPGTDQEYKGIIRHGAKMLYAFAEATVPKVTVVVRRSYGGAHIAMSIKSLGADLIYAWPSAEIAVTGPEGAVRILYRREIQNSKSPDDLIKERIAEYKKLFANPYWAAEKGLIDDVIEPKDTRKVIASALKMLKNKREFRYPKKHGNIPL

SEQ ID NO: 6 - DNA, 510 bp, Cenarchaeum symbiosum
  CDS (1)..(510)
atgaaatacgagatagaagacgcgggctccttcgagggcaggatggcggcaaaccccggaaacggagaatacacactggagataaacggaaaagaggtgcggctcaaggtaatatcgatgggcccccgcgggatggagtttctgctggaccaaaagtaccactcggcaagatacctggagaggagcacatccggcattgacatgataatcgacggaacgcccgtcagggcaggcatgcatgcagatttagacaagatagtctacaaaaattcgggcggcgggggaggcggcggccccggcattgccctgcggagccagataccaggcaaggtcgtatcattggaggtatccgagggggacgagataaagaagggcgaccccgtggcggtccttgagtcaatgaagatgcaggtggccgtcaaggcgcacaaagacggcacggtaaaatccgtcagcataaaggagggcggcagcgtcgcaaagaacgacgtcatcgccgagatagaataa

SEQ ID NO: 7 - protein, 169 aa, Cenarchaeum symbiosum
MKYEIEDAGSFEGRMAANPGNGEYTLEINGKEVRLKVISMGPRGMEFLLDQKYHSARYLERSTSGIDMIIDGTPVRAGMHADLDKIVYKNSGGGGGGGPGIALRSQIPGKVVSLEVSEGDEIKKGDPVAVLESMKMQVAVKAHKDGTVKSVSIKEGGSVAKNDVIAEIE

SEQ ID NO: 8 - DNA, 1431 bp, Cenarchaeum symbiosum
  CDS (1)..(1431)
atgatcaggacctgcagggcgctcggccttgggtcggtggcagtatactccgacgaggactataacgcgctgcacgtcaaaaaggcatccgagtcataccacataggcggggcggccccggctgaatcctacctcaaccagcagaggatcatagaggcggcgctctcctccggcgcggatgccattcacccgggatacggctttctctcggagaacggcgagtttgccgcgctgtgcgaaaagaacaggataaactttatcggcccttccgccaaatcgatgaacctgtgcggcgacaagatggagtgcaaggccgcaatgctcaaggccgatgtgcccacggttcccggcagcccgggcttggtgggcagcgccgacgaggcggccggcatagcgtcaaagataggctatcctgttctgctcaagtcggtctttggcgggggcggcaggggcatccgcctggctgaagacgagggcgggctcaggggcggatatgattctgccacagcagaatcgatagcggctgtaggcaagtcggccatactggtggaaaagttcctcaagaggacccgccatatagaataccagatggcgcgcgacaagcacggaaacgcagtccacatattcgaaagggagtgctcgatacagagaagaaaccagaagctcatcgagcagaccccctcgcctgtaatggacgaggatacccgcaagaggataggcgatctggtggtcaaggcagccgaggccgtcgactataccaacctgggaacggcagagtttttgcgcgcggattccggcgagttttatttcatagagatcaacgcgaggctgcaggtggagcaccccataacggagctggtctcggggctggacctagtcaagctgcagatagacatagcaaacggcgagcccctgcccttcaagcagaatgacctgaggatgaacggctacgccatagagtgcaggataaacgcagaagatacgtttcttgactttgcgccgtcggtcgggccggtcccggacgtcaagctgccatccgggccgggcgtgcggtgcgacacatacctgtaccctggatgcacagtctcgccgttctatgattctctgatggcaaagctgtgcacctggggggcgacattcgaggagtcaaggctcaggatgctgggcgccctcggcgacttttacgtggaaggagtggagacatccatccccctctacaagacgataatggcatccgacgagtacaaaaacggcgagctctccacggactttctctccaggtacaatatcatagacaggctggacaaagacatcaaaaaggagagggccgcaaacggcgaggctgcagcagccgccgccataatgcactcggagtttctatcgagcagggcgggcgggaacagcggaaccgcatggaagggaggcgcataa

SEQ ID NO: 9 - protein, 476 aa, Cenarchaeum symbiosum
MIRTCRALGLGSVAVYSDEDYNALHVKKASESYHIGGAAPAESYLNQQRIIEAALSSGADAIHPGYGFLSENGEFAALCEKNRINFIGPSAKSMNLCGDKMECKAAMLKADVPTVPGSPGLVGSADEAAGIASKIGYPVLLKSVFGGGGRGIRLAEDEGGLRGGYDSATAESIAAVGKSAILVEKFLKRTRHIEYQMARDKHGNAVHIFERECSIQRRNQKLIEQTPSPVMDEDTRKRIGDLVVKAAEAVDYTNLGTAEFLRADSGEFYFIEINARLQVEHPITELVSGLDLVKLQIDIANGEPLPFKQNDLRMNGYAIECRINAEDTFLDFAPSVGPVPDVKLPSGPGVRCDTYLYPGCTVSPFYDSLMAKLCTWGATFEESRLRMLGALGDFYVEGVETSIPLYKTIMASDEYKNGELSTDFLSRYNIIDRLDKDIKKERAANGEAAAAAAIMHSEFLSSRAGGNSGTAWKGGA

SEQ ID NO: 10 - DNA, 1548 bp, Cenarchaeum symbiosum
  CDS (1)..(1548)
atgcattctgaaaagcttgacaagcgctcggccaacaacaggtccgccctcatggggggcggggaggccagaatcgaggcccagcacggcaagggcaagctcaccgccagggagaggatagccatcatgctcgacgaggggagctttacggaggtggactcgctggcgacccaccactaccacgagttcgacatgcagaagaaaaagttctttggggacggggttgtcggcgggtacggcaggatagacggcaggaaggtctttgtcttcgcgtacgactttaccgtgatgggcggcacgctcagccagatgggcgcaaaaaagatcacaaagctgatggaccatgcagtaaggacaggctgccccgtgataggggtcatggattcagggggggcccgtatacaggaggggataatgagcctcgacgggtttgcggacatattctaccacaaccagcttgcatccggggtggtgccccagatcacagctagcatagggccgtcggcggggggctccgtgtactcgcccgccatgacggactttgtgataatggtcgaaaagtcggccaccatgttcgtcacggggcccgacgtggtgcagacggtcctcggcgagtccatctcgtttgaggacctcggcggcgccatgacccacgggtccaagagcggcgtggcccactttgtcgcaaagaacgagtacgattgcatggactatatcaggaagctgctctcgtttatcccccagaacaacagggaggagccgccggtagtaaagactgccgacgaccccgacaggctcgaccacggccttatcggaatgatccccgagaacccgctgcagacctacgacatgaaaaatgtgatacactcgatagtggacgaccgcacgttccttgaagtgcacgaaaactttgccacgaatatcatagtagggttcggccggttcaacggcagggccgcaggaatagtggccaaccagccggccagccttgcgggcgcgctcgacatagacgcgtccagcaaggccgcaaggttcatccggttctgcgacgcgttcaacataccggtgatcacccttgttgacaccccggggtacatgcccggctccgaccaggagcacggcgggataatccggcacggcagcaagctcctctttgcatactgcgaggccaccatccccaagataacgctggtaataggcaaggcctacgggggggcctacatagccatggccagcaagaacctgggaacggatatcaactatgcgtggcctaccgcccgctgcgccgtgctcggcgcagaagctgccgtaaagataatgaaccgaaaggacctggctgcggcatccgaccccgaggggctcaaaaaggagctgataggcaactttgccgaaaagttcgacaacccgtacgttgccgcgtcccacgggacagtggacgccgtaatagaccccgcagagacccgccccatgctgataaaggcgctcgagatgctctcgtccaagcgcgagggccgcatttccagaaagcacggaaacataaacctgtga

SEQ ID NO: 11 - protein, 515 aa, Cenarchaeum symbiosum
MHSEKLDKRSANNRSALMGGGEARIEAQHGKGKLTARERIAIMLDEGSFTEVDSLATHHYHEFDMQKKKFFGDGVVGGYGRIDGRKVFVFAYDFTVMGGTLSQMGAKKITKLMDHAVRTGCPVIGVMDSGGARIQEGIMSLDGFADIFYHNQLASGVVPQITASIGPSAGGSVYSPAMTDFVIMVEKSATMFVTGPDVVQTVLGESISFEDLGGAMTHGSKSGVAHFVAKNEYDCMDYIRKLLSFIPQNNREEPPVVKTADDPDRLDHGLIGMIPENPLQTYDMKNVIHSIVDDRTFLEVHENFATNIIVGFGRFNGRAAGIVANQPASLAGALDIDASSKAARFIRFCDAFNIPVITLVDTPGYMPGSDQEHGGIIRHGSKLLFAYCEATIPKITLVIGKAYGGAYIAMASKNLGTDINYAWPTARCAVLGAEAAVKIMNRKDLAAASDPEGLKKELIGNFAEKFDNPYVAASHGTVDAVIDPAETRPMLIKALEMLSSKREGRISRKHGNINL

SEQ ID NO: 12 - DNA, 1368 bp, Chloroflexus aurantiacus
  CDS (1)..(1368)
atgattcgcaaagtattagtagccaaccgcggtgaaattgctgtacgtatcattcgggcctgtcaggagttgggcattcgcacggtagtcgcctacagcactgccgaccgcgactcactggccgttcgtctggccgatgaagcggtatgtatcggcccaccaccggcagcaaagtcgtacctcaacgctccggctctcatcagcgctgccctcgtatcgggatgtgatgcgatccatcccggctacggttttctctcagaaaacccctactttgccgaaatgtgcgccgactgcaaactgaccttcatcggtccgccgcctgaaccgatccggctgatgggtgataaggcgattggacgtgagacgatgcgcaaagccggcgtcccaaccgtccccggctctgatggcgaagttcgctcgctcgaagaggccatcgatgtcgcccgccagatcgggtatccggtactgctcaagccctctggcggtggtggtggtcgcggcatgcgcgtcgcttacgacgaagccgatctccagcgcgccttccccactgcgcgtgccgaagccgaggcagcgttcgggaacggtgcgcttctactggaaaaatacctcacccgcgtgcgccacgtcgaaatccaggtcctggccgaccagtacggtcatgccatccacctcggcgaacgcgactgctcggcgcaacgtcgtcaccagaaaatcgttgaagaggcgccatcaccggcagtcacccccgaattgcgcgagcggatgggggccgatgctgtgcgtgggatcaaatcgattggctatgtgaatgccggcacgctcgaattcctgctcgatcaggacggcaactactacttcatcgaaatgaacacccgcatccaggttgagcatcccgtcaccgaacaggtgaccggaatcgacctcgtgcgctggcagctactcatcgccagcggcgagcgcttgacgctgcgccaggaagacattaaaataacccggcacgcaatcgagtgccggatcaatgccgaagacccggagcgtgactttttaccggcaagtggtgaagttgagttctatttgccgcccggcggccctggagttcgagtcgactcccacctctattccgggtacacaccacccggaacctacgactcattgttggcgaaaattattacctttggcgacacacgcgacgaagcgctcaaccggatgcggcgagcgctcaacgaatgcgtgattactggtattaaaacaaccatcccgttccagttggcgctgatcgacgatccggaattccgggcaggtcgcattcataccggttacgtggctgaattactgcgccaatggaaagaaacactcaatccggtataa

SEQ ID NO: 13 - protein, 455 aa, Chloroflexus aurantiacus
MIRKVLVANRGEIAVRIIRACQELGIRTVVAYSTADRDSLAVRLADEAVCIGPPPAAKSYLNAPALISAALVSGCDAIHPGYGFLSENPYFAEMCADCKLTFIGPPPEPIRLMGDKAIGRETMRKAGVPTVPGSDGEVRSLEEAIDVARQIGYPVLLKPSGGGGGRGMRVAYDEADLQRAFPTARAEAEAAFGNGALLLEKYLTRVRHVEIQVLADQYGHAIHLGERDCSAQRRHQKIVEEAPSPAVTPELRERMGADAVRGIKSIGYVNAGTLEFLLDQDGNYYFIEMNTRIQVEHPVTEQVTGIDLVRWQLLIASGERLTLRQEDIKITRHAIECRINAEDPERDFLPASGEVEFYLPPGGPGVRVDSHLYSGYTPPGTYDSLLAKIITFGDTRDEALNRMRRALNECVITGIKTTIPFQLALIDDPEFRAGRIHTGYVAELLRQWKETLNPV

SEQ ID NO: 14 - DNA, 543 bp, Chloroflexus aurantiacus
  CDS (1)..(543)
atgatgctgtggggagctatgaaggacgaaacaacagaattgccagccgatcagcccgatcctttcggtcttgctgccgttcgcgtgctcttgcaaatgctcgaacagagcgatgtctacgaaattacaattgaaaatggtaatgcgaagctgcacgtcaagcgtggtcagcccggcggtgtgatctattcggcaccactgccaacagcaccggttcccagtccatcgctaccggctacaccggtcactccatttgttcagccgccacctgcaccggaagggccgccggtcgagatgccggcaggtcatacgattactgcaccaatggtcggtacgttctacgctgctccttcgccgagagatcgaccttttgtccaggaaggcgatgaagttcgggttggtgatacggttggtatcgttgaagcaatgaagatgatgaatgagatcgagagcgatgtggccggtcgggttgcccgcattctggtcaagaatggtcagccggtcgagtatgggcaaccactgatggtgatcgaaccactctaa

SEQ ID NO: 15 - protein, 180 aa, Chloroflexus aurantiacus
MMLWGAMKDETTELPADQPDPFGLAAVRVLLQMLEQSDVYEITIENGNAKLHVKRGQPGGVIYSAPLPTAPVPSPSLPATPVTPFVQPPPAPEGPPVEMPAGHTITAPMVGTFYAAPSPRDRPFVQEGDEVRVGDTVGIVEAMKMMNEIESDVAGRVARILVKNGQPVEYGQPLMVIEPL

SEQ ID NO: 16 - DNA, 849 bp, Chloroflexus aurantiacus
  CDS (1)..(849)
atggaagaaactgctatccctcaatcgctgacgccctgggatcgcgtacaactggctcgccatccacaacggccacacacgctggattacattgctgctctgtgtgaggattttgtcgaattgcatggagatcgccgctttggcgatgacccggcgatggtcggtggaatggcaacctttgccggtcaaacggtgatggtcatcgggcatcaaaagggcaacgatacccgtgaaaatatgcggcgcaacttcggtatgccccatcccgaagggtatcgcaaagcgcaacgcttgatgcgccacgccgagaagtttggcctgccggtcatctgtttcgtcgatacaccggctgccgaccccaccaaaagttcagaagagcgtggtcaggcgaatgcgattgctgaaagcattatgctgatgacaacgctgcgtgttccgagcatcgcggttgtgatcggtgaaggtggtagtggtggcgcattggctatcagtgtcgctgaccgcattttgatgcaagagaacgccatttattccgtggccccgccagaggcagccgcctcgatcctgtggcgtgatgccgcaaaagcacccgaagcagcgcgggcattgaaactaactgccgccgatctctacgatttacggatcatcgatgaggtcatccccgaaccaccaggcggcgcccacgcagatcgtttaaccgcaattaccactgttggcgaacgtctgcgtgtgcatctggccgatctgcaacaacgcgatattgacaccctgctgcgcgaacgatatcgaaagtatcgctcgatgggtcagtaccaggaacaacaaatggatttctttggtcgaatgtag

SEQ ID NO: 17 - protein, 282 aa, Chloroflexus aurantiacus
MEETAIPQSLTPWDRVQLARHPQRPHTLDYIAALCEDFVELHGDRRFGDDPAMVGGMATFAGQTVMVIGHQKGNDTRENMRRNFGMPHPEGYRKAQRLMRHAEKFGLPVICFVDTPAADPTKSSEERGQANAIAESIMLMTTLRVPSIAVVIGEGGSGGALAISVADRILMQENAIYSVAPPEAAASILWRDAAKAPEAARALKLTAADLYDLRIIDEVIPEPPGGAHADRLTAITTVGERLRVHLADLQQRDIDTLLRERYRKYRSMGQYQEQQMDFFGRM

SEQ ID NO: 18 - DNA, 918 bp, Chloroflexus aurantiacus
  CDS (1)..(918)
atgaaagaattcttccgcctaagtcgaaaagggtttaccgggcgtgaggatcaagacagcgcccaaatccccgatgatctgtgggtgaagtgcagttcctgtcgtgagcttatctacaaaaaacagctcaacgataacctgaaggtctgccccaaatgcggtcatcacatgcgcctgagtgcccacgagtggctcggtctcctcgatgtcggttcgttccgcgaaatggatgccaatctattgccgaccgatcccctgggtttcgtcaccgacgaggagagttacgcagccaagctggcgaaaactcaacaacgcaccggcatggccgatgcagtgattgccggcattggtgccatcagcaacatgcagatctgcgtcgcagtggctgatttctccttcatgggcgcttcaatgggcagtgtctacggcgaaaaaatggcccgctccgccgaacgggctgctgaactgggtgtccctctgctcaccatcaatacatccggtggtgctcgtcagcaagagggggtgatcgggctgatgcagatggctaaggtaacaatggctctcacccggttggcggatgccggtcaaccccatattgcgttgctggttgatccctgttacggcggtgtgacggcttcgtatccttcggtggccgatattatcatcgccgaaccgggagccaacattggttttgccggcaagcgtttaatcgagcagatcatgcgccagaagttaccagccggcttccagaccgccgagtttatgctcgaacatggcatgatcgatatggtggtgccgcgaagcgagatgcgtgacacactggcccgtattctgcgtctctaccgccagcgctcaacatcacccgctaaagctgagcttgccggtcgacgagcaacgttaccgcaaccgattatgtaa

SEQ ID NO: 19 - protein, 305 aa, Chloroflexus aurantiacus
MKEFFRLSRKGFTGREDQDSAQIPDDLWVKCSSCRELIYKKQLNDNLKVCPKCGHHMRLSAHEWLGLLDVGSFREMDANLLPTDPLGFVTDEESYAAKLAKTQQRTGMADAVIAGIGAISNMQICVAVADFSFMGASMGSVYGEKMARSAERAAELGVPLLTINTSGGARQQEGVIGLMQMAKVTMALTRLADAGQPHIALLVDPCYGGVTASYPSVADIIIAEPGANIGFAGKRLIEQIMRQKLPAGFQTAEFMLEHGMIDMVVPRSEMRDTLARILRLYRQRSTSPAKAELAGRRATLPQPIM

SEQ ID NO: 20 - DNA, 1116 bp, Ricinus communis
  CDS (1)..(1116)
atgttaaaagtaccttgttgtaatgctacagacccaattcaatccctatcttcccaatgcagattcctaacccatttcaataacagaccttatttcactcgccgcccatcaatccctacctttttcagttcaaagaattcaagtgcttctcttcaggctgttgtgtctgatattagcagtgttgaatctgctgcttgtgacagtttggcgaatcggcttcgtttagggaagttaactgaggatgggttctcttataaagagaaatttattgttaggagttatgaggttgggattaataaaactgctactgttgaaactattgctaatctactgcaggaggttggatgtaatcatgctcagagtgttggattttcaaccgatggatttgccacaaccaccagcatgaggaagatgcatctgatatgggtaactgctcgcatgcatatcgaaatatacaaatatcctgcctggagcgatgtagttgaagtagaaacatggtgccaaagtgaaggaagaattggaaccagacgtgattggattttgacagattatgccactggtcaaattatagggagagccacaagcaagtgggtgatgatgaaccaagacactagacggcttcagaaagtcactgatgatgtccgagaagagtacttagttttctgcccaagagaacttagattggcatttccagaggaaaacaatcgcagctcgaagaaaatttcaaaactagaagatcctgctcaatattccaagctaggactagtgcctagaagagcagatctggacatgaatcaacatgttaataacgtcacctacataggttgggttctagagagcatacctcaagaaataattgacacacacgaactacaaacaatcactttggattacagaagggaatgtcaacatgatgatattgttgattccctcacaagtgtggaaccttctgagaatttagaagctgtttcagagcttcgagggacaaatggatctgccactacaacggctggtgatgaagactgccgtaactttctacatctactcaggttgtcaggtgatgggcttgaaataaaccgtggccgcactgaatggagaaagaaatctgcgagatga

SEQ ID NO: 21 - protein, 371 aa, Ricinus communis
MLKVPCCNATDPIQSLSSQCRFLTHFNNRPYFTRRPSIPTFFSSKNSSASLQAVVSDISSVESAACDSLANRLRLGKLTEDGFSYKEKFIVRSYEVGINKTATVETIANLLQEVGCNHAQSVGFSTDGFATTTSMRKMHLIWVTARMHIEIYKYPAWSDVVEVETWCQSEGRIGTRRDWILTDYATGQIIGRATSKWVMMNQDTRRLQKVTDDVREEYLVFCPRELRLAFPEENNRSSKKISKLEDPAQYSKLGLVPRRADLDMNQHVNNVTYIGWVLESIPQEIIDTHELQTITLDYRRECQHDDIVDSLTSVEPSENLEAVSELRGTNGSATTTAGDEDCRNFLHLLRLSGDGLEINRGRTEWRKKSAR

SEQ ID NO: 22 - DNA, 2380 bp, Arabidopsis thaliana
  CDS (103)..(2109)
agtttgtatctttgaacaaacgaaactagaaaacgcctatctctgctcattggcttctccatctccgatcaaatccgcggtcgtagtcagagagagagacaaatggctatgcctagggcaacctctggtcccgcttacccggagaggttctacgctgctgcatcttatgtcggtctcgacggatctgattcgtcggcgaaaaacgtcatctcgaaattccccgatgatactgcactacttctctatgcgctataccagcaggctactgtaggaccatgcaacactccgaaacctagtgcctggagaccagtggagcaaagcaaatggaaaagttggcaggggctcggaaccatgccctccattgaggcaatgcgtctctttgtgaaaattctggaggaagatgatcctggttggtattcgagggcatctaatgatattccagatcctgttgtagatgtccaaattaatagagcaaaagatgagcctgttgttgagaatgggagcacatttagtgagacaaaaacaatttctacggagaatggacgtttggctgaaacccaagataaagatgtagtctcagaagactcaaatactgtttctgtatataaccagtggactgcaccccaaacatcaggtcagcgtccaaaagctcgttacgagcatggcgcagcagttattcaagataagatgtatatatatggtggaaatcacaatggccgttaccttggtgatcttcatgttctagatttaaaaagttggacttggtcaagagttgaaaccaaggttgcaacagaatctcaggaaacatcaactccgacattgttagctccttgtgctggtcattctttgatagcatgggacaacaagctgctgtctatcggtggtcacacaaaggatccctcagaatctatgcaagtgaaggtctttgatccccataccattacatggtcaatgttaaagacatatggaaaaccaccggtttcacgtggaggccagtcagttacaatggtgggtaaaaccttggtgatatttggcgggcaagatgcaaagagatcacttctgaacgatttgcatatacttgacctagacacgatgacctgggatgagatagatgccgtgggtgtatctccatctccgaggtctgatcatgctgctgcagtgcatgcagaacggttccttcttatctttggtggcggctcacatgcaacctgtttcgatgacctgcatgtccttgatttgcaaactatggagtggtcaagaccagcacagcaaggtgatgcaccaactccaagagctggacatgctggcgtgacaattggggagaactggtttattgttggtggcggtgataacaagagtggggcatcggagagtgttgtactaaacatgtcaactcttgcatggtcggtcgttgcttcagttcaaggacgtgtacctcttgctagcgagggattaagtttagtggtgagttcatacaatggtgaagatgtactagtcgcttttggtggatacaatggacgttacaataacgaaattaatcttcttaaaccaagccacaaatcaacattgcaaacaaagactctggaagcgcctttgccaggtagtctttctgctgttaataatgccacaaccagagacattgagtctgaggttgaggtgagccaagaaggcagggtacgggaaattgtcatggacaatgttaaccctggatcaaaggttgaaggaaacagcgaacgcattattgcgactattaaatctgagaaggaagagctggaggcatcactgaacaaagagaggatgcagactctccaactaaggcaagagttaggagaagcagaattacgaaacacagatttgtacaaggaacttcaatctgttcgtggccaacttgcagccgaacaatcaaggtgtttcaaactggaggttgatgttgcagagctaagacaaaagcttcaaacattggaaacactacagaaggaactcgaactcctgcaacgtcaaaaggctgcctcagaacaagctgcaatgaacgctaaacgacagggctcgggtggcgtatggggctggctcgctggaagccctcaggaaaaagatgatgattcgccttgatcatttttggtccggtaatgccattttcctccggacgatatttgctttcagtaaagaaatatatatagttttttttgtctgagtctctcgcattgtttcttcttccaataagatatttttttagcgagctttgattactaccgatgtttagctttgttctctgttatacaagtgattcaccagaatattcgttaacttttttgaaatatttgttttgggagtgtctgacaaaattatgaaaactaaatcagtttacgatcagcaatcataa

SEQ ID NO: 23 - protein, 668 aa, Arabidopsis thaliana
MAMPRATSGPAYPERFYAAASYVGLDGSDSSAKNVISKFPDDTALLLYALYQQATVGPCNTPKPSAWRPVEQSKWKSWQGLGTMPSIEAMRLFVKILEEDDPGWYSRASNDIPDPVVDVQINRAKDEPVVENGSTFSETKTISTENGRLAETQDKDVVSEDSNTVSVYNQWTAPQTSGQRPKARYEHGAAVIQDKMYIYGGNHNGRYLGDLHVLDLKSWTWSRVETKVATESQETSTPTLLAPCAGHSLIAWDNKLLSIGGHTKDPSESMQVKVFDPHTITWSMLKTYGKPPVSRGGQSVTMVGKTLVIFGGQDAKRSLLNDLHILDLDTMTWDEIDAVGVSPSPRSDHAAAVHAERFLLIFGGGSHATCFDDLHVLDLQTMEWSRPAQQGDAPTPRAGHAGVTIGENWFIVGGGDNKSGASESVVLNMSTLAWSVVASVQGRVPLASEGLSLVVSSYNGEDVLVAFGGYNGRYNNEINLLKPSHKSTLQTKTLEAPLPGSLSAVNNATTRDIESEVEVSQEGRVREIVMDNVNPGSKVEGNSERIIATIKSEKEELEASLNKERMQTLQLRQELGEAELRNTDLYKELQSVRGQLAAEQSRCFKLEVDVAELRQKLQTLETLQKELELLQRQKAASEQAAMNAKRQGSGGVWGWLAGSPQEKDDDSP

SEQ ID NO: 24 - DNA, 641 bp, Arabidopsis thaliana
  CDS (106)..(384)
aggtttatcatgacgcccatatatatctcacgcgttgtcctcgtcttctccgtcttacactgatttaattctcctaccaatctcaacttccgacgtctattcatcatgggtttgaaggaggaatttgaggagcacgctgagaaagtgaatacgctcacggagttgccatccaacgaggatttgctcattctctacggactctacaagcaagccaagtttgggcctgtggacaccagtcgtcctggaatgttcagcatgaaggagagagccaagtgggatgcttggaaggctgttgaagggaaatcatcggaagaagccatgaatgactatatcactaaggtcaagcaactcttggaagttgctgcttccaaggcttcaacctgatgaatcaaatcctcatctgcagtaactttatcttaagcatcaaaataacattgcataagacttgttcttgctcttgtgtttctatcatatttaagctatctactttgtgacatggtgtgatctcttaaaaatgcttgatattggttaaaacagagaatcatgatgcaaactaaatccataagttatttttggtccgtcctcgatatggtcttagttaaaacagttgaattcaagatgatatattcgttctggtccgt

SEQ ID NO: 25 - protein, 92 aa, Arabidopsis thaliana
MGLKEEFEEHAEKVNTLTELPSNEDLLILYGLYKQAKFGPVDTSRPGMFSMKERAKWDAWKAVEGKSSEEAMNDYITKVKQLLEVAASKAST

SEQ ID NO: 26 - DNA, 18 bp, Artificial Sequence
  forward primer for cloning of B. napus ACP
gcggccaaaccagagacg

SEQ ID NO: 27 - DNA, 43 bp, Artificial Sequence
  reverse primer for cloning of B. napus ACP
tcagtggtggtggtggtggtgcttcttggcttgcaccagctct

SEQ ID NO: 28 - DNA, 24 bp, Artificial Sequence
  forward primer for cloning of B. napus BC
ttggtgaagctcctagcaaccagt

SEQ ID NO: 29 - DNA, 24 bp, Artificial Sequence
  reverse primer for cloning of B. napus BC
ttcttcatcgtctccctggcagtt

SEQ ID NO: 30 - DNA, 24 bp, Artificial Sequence
  forward primer for cloning of B. napus BCCP
agtgactaacggtgggtgcttgaa

SEQ ID NO: 31 - DNA, 24 bp, Artificial Sequence
  reverse primer for cloning of B. napus BCCP
tgataaactggagctggtggtggt

SEQ ID NO: 32 - DNA, 24 bp, Artificial Sequence
  forward primer for cloning of B. napus CT- alpha
tacgtgacagctcgcctcaagaaa

SEQ ID NO: 33 - DNA, 24 bp, Artificial Sequence
  reverse primer for cloning of B. napus CT- alpha
caaaccagtttcagccgccatctt

SEQ ID NO: 34 - DNA, 24 bp, Artificial Sequence
  forward primer for cloning of B. napus CT- beta
ggagcacgaatgcaagaaggaagt

SEQ ID NO: 35 - DNA, 24 bp, Artificial Sequence
  reverse primer for cloning of B. napus CT- beta
acatacccaaacttgctgtcaccc

SEQ ID NO: 36 - DNA, 96 bp, Ricinus communis
atggctctcaagctcaatcctttcctttctcaaacccaaaagttaccttctttcgctcttccaccaatggccagtaccagatctcctaagttctac

SEQ ID NO: 37 - protein, 32 aa, Ricinus communis
MALKLNPFLSQTQKLPSFALPPMASTRSPKFY

SEQ ID NO: 38 - DNA, 165 bp, Arabidopsis thaliana
atggcttcctctatgctctcttccgctactatggttgcctctccggctcaggccactatggtcgctcctttcaacggacttaagtcctccgctgccttcccagccacccgcaaggctaacaacgacattacttccatcacaagcaacggcggaagagttaactgc

SEQ ID NO: 39 - protein, 55 aa, Arabidopsis thaliana
MASSMLSSATMVASPAQATMVAPFNGLKSSAAFPATRKANNDITSITSNGGRVNC

SEQ ID NO: 40 - DNA, 180 bp, Artificial Sequence
  synthetic chloroplast targeting sequence based on Rubisco consensus sequence
atggcttcttctatgctttcttctgctgctgttgttgctactcgtgctagtgctgctcaagctagtatggttgctccttttactggacttaagtctgctgcttcttttcctgttactagaaagcaaaacaaccttgatattacttctattgctagtaacggaggaagagtccaatgcgca

SEQ ID NO: 41 - protein, 59 aa, Artificial Sequence
  synthetic chloroplast targeting sequence based on Rubisco consensus sequence
MASSMLSSAAVVATRASAAQASMVAPFTGLKSAASFPVTRKQNNLDITSIASNGGRVQC

SEQ ID NO: 42 - DNA, 174 bp, Artificial Sequence
  Solanum tuberosum rubisco chloroplast targeting sequence - codon optimized
atggcttcctctgttatctcttcagctgctgttgccaccagaacaaacgttacgcaagctggatcgatgatcgcaccgttcactggacttaagtctgcagctacgttcccagtttccaggaagcaaaacctcgacatcacctcaatcgccagtaacggtggaagagtcagatgt

SEQ ID NO: 43 - protein, 58 aa, Artificial Sequence
  Solanum tuberosum rubisco chloroplast targeting sequence - codon optimized
MASSVISSAAVATRTNVTQAGSMIAPFTGLKSAATFPVSRKQNLDITSIASNGGRVRC

SEQ ID NO: 44 - DNA, 372 bp, Artificial Sequence
  transit peptide based on Zea mays and Helianthus annuum Rubisco sequence
atggccagtatcagttcctctgtcgctacagtttctcgaactgcaccagctcaggcaaacatggtagctccctttactggtctcaaaagcaacgctgcctttccaacaactaagaaagccaacgacttctcaacacttccatccaacggcgggagagttcaatgcatgcaagtatggcccgcttatgggaacaagaaattcgagacattgtcctatctgcctccgctttcaatggcacctactgtgatgatggcaagctctgctacagcagtcgcaccctttcaaggtttgaaaagcacagcttctttgcctgtcgctcgaaggagttctcgtagccttggtaacgtttctaacggcggtcgcattaggtgt

SEQ ID NO: 45 - protein, 124 aa, Artificial Sequence
  transit peptide based on Zea mays and Helianthus annuum Rubisco sequence
MASISSSVATVSRTAPAQANMVAPFTGLKSNAAFPTTKKANDFSTLPSNGGRVQCMQVWPAYGNKKFETLSYLPPLSMAPTVMMASSATAVAPFQGLKSTASLPVARRSSRSLGNVSNGGRIRC

SEQ ID NO: 46 - DNA, 504 bp, Sulfolobus metallicus
atgaagctcctcagagtttacatggaaactggagaaacctacattgcaagctatgaccagaaggataacaaggacacagtgaaaatggaagaaggagagttaaatgtagaattcttaggtagaggtacaagagaaaacgagtacttgttcaaggttggaaacgaagtacacagcattacaattgataggggattcctaatattagatcaggaagaagagtataaggtagacaggataacagaacttccagtaaaggagggtcaatcagtagaggaattaatgaaaggaaaagaaggagaagtattatcgccattgcaaggtagagttgtagctataagagtaaaggaaggtgacgcagtaactaaaggtcagcctcttctatctgtagaagcaatgaaatctgagaccataatatctgcaccaatagctggagttatagagaaaattgcggtaaaaccagggcagggagtaaagaaaggagacttgctggttgtactaaaataa

SEQ ID NO: 47 - DNA, 504 bp, Acidianus brierleyi
atgaaactatatagggcatacgcagatactggagatacctatataatggctatagatagtaaaggtgataaagataagataaaaacagagaataatgaatttgaaatagaatatctgggaaaaggtacaagagaaaatgaatatctattcaaagttaatggaaaagttcatagagcattcatagacaatggctatatactcttagataacgctagcgtatttagattagaaaggcttacagaacttccttcaaaagaaggcgagtcaatagaagaaatgatcaaaggaaaggaaggtgaagtaatatcaccattacaaggcagaatagttacgattagggttaatgaaggcgatgcagtaaataaaggccaacctttgctttctgttgaagctatgaaatctgagacaattatttccgcgccaatagcaggaatagtagagaaaattattgtaaaacctggacaaggagtaaagaaaggagatactctacttataattaaataa

SEQ ID NO: 48 - DNA, 510 bp, Sulfolobus tokodaii
gtggtaatgaagttacttagggtatcttcagaattaggagatagctatgtaatgacttacgatcaacaaggtaataaagataccgtcagctttgaggataataagtttgaaatagaatatattggacccggatggagagaaggagaactactcttcaagattaatggtgaggttcatagagtatacgtagacaatggttttattgtaatagatgatgaaacgatctttaaggtagatagaataacggaaacaccaatagaacaaggtaagtcgattgaagagttgataaaaggaaaagaaggcgaaattttatcaccaatgcaaggaagaattgttcagataagagtaaaagaaggcgatgctgtaaacaaaggacagccattattatctattgaggcaatgaagagtgagactgtaatatccgccccagtaggtggagtagtacagaaaattatggtaaaacctggtcagggtgtaaagaaaggtgatttattacttattattaaatga

SEQ ID NO: 49 - DNA, 504 bp, Acidianus hopitalis
atgaagttatacagagtctattccgagactggagatacttacttgatggcagttgaaagtaaaggcaacgttgataagataaaaactgaagctaatgagtttgagatagagtatttaggaaaaggtactagagataacgagtacctatttaaggtaaatggtgaaatccatcatgtatttgtagataatggatatgtctttgttgacaacgctagtgtatttaagttagagagagctacagaattgccaactaaggaaggtgaatcagttgaggaaatgattaagggaagagaaggagaagttatttcgccattacaaggaagggttgttagtataagagttaaagaaggtgatgctgtgaacaaaggtcaacctttattatctatcgaggcaatgaaatctgaaactataatttctgctcctgttgcaggagttgtagagaaaatcctagttaagcaaggtcaaggagtaaagaagggagatacacttgttatcattaagtga

SEQ ID NO: 50 - DNA, 504 bp, Metallosphaera sedula
atgaaactgtatagggttcatgcggatacaggagataccttcattgtggcccacgatcaaaaggaaaacaaggacagactaaagacggaaaataacgagtttgagatagagtatgtcggtcagggtacaagggaaggagaaataatcctgaagattaacggtgagatgcacagggtcttcatagacaacggatggataattcttgacaatgcaaggatattcagggcagagagagttacagagcttcccactcaggaaggacagacactggacgagatgatcaaaggtaaggagggagaagtgctatcaccgcttcagggcagagtagttcaggtcagggttaaggaaggcgatgcggtgaataagggacagcccttgctatcgattgaggccatgaaatctgagaccatagtgtcggcaccaataagcgggctagtggagaaggtattagttaaggcaggtcaaggagtaaagaagggagatatcctagtggtgataaagtaa

SEQ ID NO: 51 - DNA, 504 bp, Metallosphaera cuprina
atgaagctttacagagtacactcagaggtcggagatacattcatcatagctcaggaccagggccagaacaaagataaggttaagactgagaataatgagtttgagatcgagtacgttggacagggaaggagagagggagaaataattctaagggttaatggagaagaacatagggcggtaatagataatggatggattgttctcgacaacgccaagatatttagagccgaaaggataacagagcttcctacgcaagagggacaatctcttgaagaaatgataaagggtaaggaaggggaggttgcatcacctttgcaagggagagtagtacagataagagtaaaggagggtgatgcggtaaacaagggacaacctcttctctcaatagaagcgatgaagtccgaaactgtgatatcggcacctattagcgggattgtggagaagatactcgttaagtccggacaaggagttaagaagggagacatcctgatagtaattaaataa

SEQ ID NO: 52 - DNA, 504 bp, Sulfolobus acidocaldarius
atgaagattataaaagtagttacagatcagggagattcttacacttttgctacagagaagagggataataaggatttaatgaaagcggagggagcagaatttgaggttgaatacctgggagctggatggagagagggagaacatttagtaaaagtcaatggcgaagtacatactgtctccataattaatggtcatttagttattgataacgagacgttattcaaagttgatagggttattgaggaatctctaggtgaaaaagtgtcatttgaggaattatttaagggtaaggagggagagatcgtttcacctctacagggtagaattgtccagataagggtaaaggagggagatgcagtaaataagggtcagcccttattatcgattgaggctatgaagagtgaaacagtaatatcagcacctaaaggaggagttgtgaaaaaagttttgataaaacctggacagggcgttaagaagggagaccttctcttgataattgaatag

SEQ ID NO: 53 - DNA, 561 bp, Sulfolobus solfataricus
ttggaaaacttatggtatcatattccaatcctctccaagggtgttgtggtaatgagattatttagactttatagcgaattgggagatatgtatttagtatcttatgaacagagcaacaacagtcttgataaaataaaaataggagataaaaattatgaggtaaaatatctaggatcaggcaatagggaaaatgagtatttatttgagattaatgggaaaaaatattatgttttcatagagtctgatggtactttgatatttaaccatcaggatttcttaaggttagataaggtaactgaaattcccataaaaggagaagaaagagttgaggaaataattagaggaaaggaaggagaaattgtatctccattatttggaagggtagttaagattagagtaaaggaaggggatgcagtaaataagggtcaacctttgttatccattgaggcaatgaaggcagaaacggtaatctcatcgccaataggtgggatcgtgcaaaaaattctgatcaaggaaggacaaggtgtaaagaagggagatattttaattgttataaaatag

SEQ ID NO: 54 - DNA, 510 bp, Sulfolobus solfataricus
atgaggttatttagaatttatagtgaattaggagatacgtttctagtatcttatgatcaaatcggtaataatgtcgataaaataaaaataggagataatatttacgaggtaaaatatctagggcccggcaatagggaaaacgaatatttattcgaagttaatggtaaaaaatactatgtctacatggaacaagatggcactttaatattcaacaatcaagatttcttaaggttagataaggtaactgaaatcccggtaaaaggagaagaaagggttgaagagattataaggggaaaagaaggagaaatagtatcgccattatttggaagggtagttaaaataagagtaaaagaaggagatgcagtaaataagggtcaacctttgttatcaatcgaggcaatgaaggcagaaacagttctctcatcgccaataggtgggatcgtgcaaaaaattcttgtcaaagaagggcaaggtgtaaaaaagggagatattttagtcgttataaaataa

SEQ ID NO: 55 - DNA, 510 bp, Sulfolobus islandicus
atgagattatttagactttatagcgaattgggagatatgtatttagtatcttatgaacagagcaacaacagtcttgataaaataaaaataggagataaaaattatgaggtaaaatatctaggatcaggcaatagggaaaatgagtatttatttgagattaatgggaaaaaatattatgttttcatagagtctgatggtactttgatatttaaccatcaggatttcttaaggttagataaggtaactgaaattcccataaaaggagaagaaagagttgaggaaataattagaggaaaggaaggagaaattgtatctccattatttggaagggtagttaagattagagtaaaggaaggggatgcagtaaataagggtcaacctttgttatccattgaggcaatgaaggcagaaacggtaatctcatcgccaataggtgggatcgtgcaaaaaattctgatcaaggaaggacaaggtgtaaagaagggagatattttaattgttataaaatag

SEQ ID NO: 56 - DNA, 510 bp, Sulfolobus islandicus
atgaggttatttagactttatagcgaattgggagatatgtatttagtatcttatgaacagagcaacaacagtcttgataaaataaaaataggagataaaaactatgaggtaaaatatctaggaccaggcaatagggaaaatgagtatttatttgagattaatgggaaaaaatattatgttttcatagagtctgatggtactttgatattcaaccatcaggatttcttaaggttagataaggtaactgaaattcccataaaaggagaagaaagagttgaggaaataattagaggaaaggaaggagaaattgtatctccattatttggaagggtagttaaaattagagtaaaggaaggggatgcagtaaataagggtcaacctttattatccattgaggcaatgaaggcagaaacggtaatctcatcgccaataggtgggatcgtgcaaaaaattctgatcaaggaaggacaaggtgtaaagaagggagatattttaattgttataaaatag

SEQ ID NO: 57 - DNA, 510 bp, Sulfolobus islandicus
atgagattatttagactttatagcgaattgggagatatgtatttagtatcttatgaacagagcaacaacagtcttgataaaataaaaataggagataaaaattatgaggtaaaatatctaggatcaggcaatagggaaaatgagtatttatttgagattaatgggaaaaaatattatgttttcatagagtctgatggtactttgatatttaaccatcaggatttcttaaggttagataaggtaactgaaattcccataaaaggagaagaaagagttgaggaaataattagaggaaaggaaggagaaattgtatctccattatttggaagggtagttaagattagagtaaaggaaggggatgcagtaaataagggtaaacctttgttatccattgaggcaatgaaggcagaaacggtaatctcatcgccaataggtgggatcgtgcaaaaaattctgatcaaggaaggacaaggtgtaaagaagggagatattttagttgttataaaatag

SEQ ID NO: 58 - DNA, 510 bp, Sulfolobus islandicus
atgaggttatttagactttatagcgaattgggagatatgtatttagtatcttatgaacagagcaacaacagtcttgataaaataaaaataggagataaaaactatgaggtaaaatatctaggaccaagcaatagggaaaatgagtatttatttgagattaatgggaaaaaatattatgttttcatagagtctgatggtactttgatattcaaccatcaagatttcttaaggttagataaggtaactgaaattcccataaaaggagaagaaagagttgaggaaataattagaggaaaggaaggagaaattgtatctccattatttggaagggtagttaaaattagagtaaaggaaggggatgcagtaaataagggtcaacctttattatccattgaggcaatgaaggcagaaacggtaatctcatcgccaataggtgggatcgtgcaaaaaattctgatcaaggaaggacaaggtgtaaagaagggagatattttaattgttataaaatag

SEQ ID NO: 59 - DNA, 378 bp, Aciduliprofundum boonei
atgagaagaaaatttaaagtaatggttaacggaaaagagtatgtagtggaaattgaagaactcggagagcccaactcgcaagcacccatacagccaaggtacgaaataaagcctgaatcttcaaagcccagtacaccaaaacctgcagagacttctgcggaagagggtgctgtcacatctcctatgcccggcaagattttagatatcagagttagcaaaggagataaagtgaagataggagatgttcttataatattagaagctatgaagatggaaaacgaaattgttgcaccaaaggatggtatagtgaaagaagtaagggtaaatgtaggagataaagtagatagaggttcagtactaattgtgataggtgaatga

SEQ ID NO: 60 - DNA, 546 bp, Chloroflexus aggregans
atgctgtggggagctatgaaggacgaaactactgcaatgccagccgatcaaaacgatccattcggcctttccgctgtgcgagatttacttcagatgctcgaacagagcgatgtctacgagatcacgatagaacgtggtaactccaagctgcacgtgaaacgtggtcaaccaaccggggtgatctattccgcaccaatgagccagcccgtacctgctcccattgccacaccactaccaactgcgcctgtcaccccattcgtgcaaccaccgcctgccccagaagggccaccggttgaaatgcccgccggtcatacgatcacggcgccaatggtcggtacattctacgcagccccatcgccaaaggataagccgtttgtgcaagaaggtgatgaggtgcgtgttggtgataccgtcggtatcatcgaagcgatgaaaatgatgaacgaaattgagagcgatgttgccggacgggtagcgcgtattttggtcaagaatggtcaaccggttgagtacggtcaaccgttgatggtgatcgaaccgctttga

SEQ ID NO: 61 - DNA, 528 bp, Oscillochloris trichoides
atgatcaatgcgacgaatacggaatcttccgagaacgctgatgactttggtctgagtgcggtacgtgaactgctgcgcttgatgaaccagaccgacattaccgagatcctgatcgagcgtggcgataccaaactgcatgtcaagcgcgggacgaccgtgcagattgcggcagtgccccatgcgcccgtggcccagacgctggctccgacggtggccgcgatggccccgcatccgatgccgatgcctgtggcagcggctccggccccagccgaggttgcggttccggctgggcatacgatcactgcaccgatggtggggacgttctacgcatcaccctcgcctaaggatgcgccctttgttcaagagggggatagcatccaggtcggtgattcggtcggtatcatcgaggccatgaagatgatgaacgagatcgagagtgatgtggccggtcggatcatccgcattctggttacgaatggccagccggttgaatatggccagccgcttatggtggtggagccggtataa

SEQ ID NO: 62 - DNA, 549 bp, Roseiflexus castenholzii
atgaacgaaggcgagcgcgatctgaccatatcaatcgaggaggagttcggtctgagcgctgtgcgcgaactgttgcggatcatcagcgaaacggacgtcagcgaaatctcgattgagcgtggaacgacacgactccacatcaaacgtggaccgtctctccatcaccattcgccggcgccgatgttcatcacgccatcgaccgccgcccatgtgcagccatcggcgccgcccatcggcatggtgcaggcgccggtgcagacgccgccgcctgtcacacctgctccagaaccggatgcattgccgccaggaaacatcattgcggcgccgatggttggaacgttctatgcagcgccatcgccgaaggatccgccctatgtccaggaaggtgatgtcattcatgtcggcgaccgggttggcattattgaagccatgaagatgatgaacgagatcgaaagcgaattcgccggtcgggtggcgaagattctggtccagaatgcacaaccggtggagtacggacaaccgttgatggtgatcgaaccgttgtcctga

SEQ ID NO: 63 - DNA, 549 bp, Roseiflexus sp
atgagcgaaggtgagcgcgatcacactgtgcagtcgatcgaggacgagttcggcttgagcgccgtgcgcgaactactgcgcatgatcagcgacacggatgtgaatgagatctcgattgagcgcggcacgacacgcctgcatatcaagcgaggtccgtcgctgcatccccatacgccggcgccgatgttcgtcacaccatcgattgccgcaaacgtgcaatcttcggcgccgccgatcggtatcgttcaggcgccggtgcagacgccgccgccggtaacacccgctccggagccggacgccctcccgccgggcaagatcatcgctgcgccaatggtcggaacattctatgccgcgccgtcgccgaaggatccgccctacgtgcaggaaggcgacatcattaatgtcggtgatcgggtcggcattatcgaggcgatgaagatgatgaacgagatcgagagcgagtttgccggtcgggtcgtgcgtattctggtgcagaatgcgcaaccggtggagtatgggcagccattgatggttatcgagccgctgtaa

SEQ ID NO: 64 - DNA, 468 bp, Herpetosiphon aurantiacus
atgagtgagcaaacgcccgaacagaatttactcaccgaggatgtgcgtgagttgctgcggttaatcacccaaactgatattactgagctaagccttgagcgtggcgatgccaaaattcatgttaagcgcacgccctatgcagtggcagcaccagtggttgtgaccagtggcgtagcggcgacaccagttgctgccaacgttgccgaaacacccgccgcgcccattggccaaccaatcacttcgccaatggtaggaaccttctatgcctcaccatcgcccaaagacccaccgtatgtcaaagttggcgatgaggttcacccaggcgatgtcgttggcatcgttgaagccatgaagatgatgaacgaaatcgagagcgaaattcatggccgcgtggcagcgatccacgttgaaaataaccaacctgttgaatatggtcaagtgctcatttcaatcgtgccgctcgattaa

SEQ ID NO: 65 - DNA, 513 bp, Nitrosoarchaeum limnia
atggattacaaaataaaagatattgaaaaaacatttgacggcgaaataactcaaagtcttggaaataatgaatatgtgataaaaatcaatgatgctgaacatcaaattaaaattctaaaaatggattcaaaaggaattgaatttgtgttagatcaaaaatatcacagagcaaaatatcttgaaaattcaacaaatgaaatgaatctcattattgataacgtgccaattacaatcaacatgcatactgatctagataaaattgtcttcaaacattctggaggtgcaagctcttctgatacccaattggcattaaagagtcaaattcctggaaaagttgtttcaattgctgtacaagaaggtgattctgtaaaacaaggagatgttgtttgtactttagaatctatgaaaatgcaagttgctataaaatctcacaaaaatggctctatcaaatctattaaaatcaaaattggtggaacagtagcgaaaaacgatcttgttgccgaaatagaataa

SEQ ID NO: 66 - DNA, 513 bp, Nitrosopumilus maritimus
atggactataagatagctgatgttgaaaaatcatttgaaggaaaaattactgaaaacttaggtaataatgattatgtgattaagatcaatgacaaagaacatcaattgaaaatccttagcatgaatgcaaaaggtatagaattcattttagatcaacaatatcataaagcaaaatatcttgagactgcaactaatgaaatgaatcttgtaattgataatgttccagttacattaaacatgaatactcactttgacgaaatagtttacaaaaactctggcggtggtggcgcaggtggtgctcaagtagcactcaaaagtcaaatccctggtaaagttgtgtcaattgcagttgctgaaggtgactctgtcaagaaaggtgatgtagtttgtactttagaatcaatgaagatgcaagttggaataaaggcacacaaagatggtgaagttaaaaatctcaaaattaaagaaggtgcaactgtcgcaaaaggcgacgttattgcagatttagaataa

SEQ ID NO: 67 - DNA, 513 bp, Group I marine crenarchaea
atggaatttaaacttgaggatatagaggaaactttcaatggagaaattattaataaaatttcaaacaacgaatatctaattaagattcaagataaagaataccaccttcaaattctgaacatcaattctcgtggaatggaatttttactggataatcattttcacagtgtaaactatatagaaaatcaaaccgcagaaatgaaaattgttgttgacggtgtgccacttacagtcaacatgcatacgaaattagacgagattgtttacaggaatacaggtggtgctgatattggttcaacacagattaatctgagaagtcaaattcctggaaaagtagtttctatagaagttaaagttggtgataaagtcaaaaacggtgatgttgtgtgtgttttagagtcaatgaagatgcaagtctccgtcaaatcacacaaagacggtgaagtaaaaaatttaaaaattaaggaaggagactctgttaacaaaaatgacatcttggctgaaattgaataa

SEQ ID NO: 68 - DNA, 513 bp, Group I marine crenarchaea
atggaatttaaacttgaggatatagaggaaactttcaatggagaaattattaataaaatttcaaacaacgaatatctaattaagattcaagataaagaacaccaccttcaaattctgaacatcaattctcgtggaatggaatttgtactggataatcattttcacagtgtaaactatatagaaaatcaaaccgcagaaatgaaaattgttgttgacggtgtgccactgacagtcaacatgcatacgaaattagacgagattgtttacaggaatacaggcggaactgatactcgttcagcgcagattaatctgagaagtcaaattcctggaaaagtagtttctataggagttaaagttggtgataaagtcaaaaatggtgatgttgtgtgtgttttagagtcaatgaagatgcaagtctccgtcaaatcacacaaagacggtgaagtaaaaaatttaaaaattaaggaaggagactctgttaacaaaaatgatatcttggctgaaattgaataa

SEQ ID NO: 69 - DNA, 504 bp, Hippea maritima
atgtatatagctacgcttgataatgtagaatacaagatagatgtaaaagagatagagcctaataaatttgaagtgataatagatgagaagtcttacatagtagatgcacagttgacagaaagctcagtttattctctaataattaacggtaaatcgtatgaggttaatttagattacaaagacggtctttattatgtttataacgagggagatctctttaaaatagaggttatggatgagctcaaaaagaggatgctcgaaaagaggggtggcgccggtggccttgagggtgcatacacgcttaagtctgaaatgcccggaaaaattattgatgttaaagtcaacgaaggtgatgaggttaaagaaggtgatattgttttgattcttgaggctatgaagatgcaaaacgagattagatcacccaaagatggtaaggttacagaggtatttgttgaggccggagaggttattgaagctgaagcaaaacttgtgacgatagagtaa

SEQ ID NO: 70 - DNA, 495 bp, Croceibacter atlanticus
atgaccaatacttataaactctcggttaatgatgagtttgagtttcagttagatgctcaagacgtcaattccctagataccagcttacaacccaataacaaacaacatattttagaagattctgcctcccaatctgtacatatagaaggtaaagatgtacttaatcgccaatatacagttcgtataaatggtaatagatatcaggtagccattaaaaatgatctcgatcttctaattgaagaaatgggattaagcttaggagcagatgctatagagaatgatatttttgcaccaatgccaggtgttattttaagtgtagatgtaaaggaaggagacagtgtaaaagaaggcgatacactatgcgtgctagaagctatgaagatggaaaacgcactgctttcaccaagagatggtgttattaaatctattgaagttactacagcagacaccgttgagaaaaatgccttattgcttaccctagaaccattatcatga

SEQ ID NO: 71 - DNA, 1542 bp, Acidianus hospitalis
atggttaaaatgcctccttttggaaaagtcctcgttgcgaatagaggagagattgcagtcagagtaatgaaggcaataaaagaaatgggaatgaaagcggttgcagtttactccgaagccgacaagtacgctttacatgttaaatacgcagatgaagcttattacataggtaaagcgccagcccttgatagttatttaaatatcgatcatattatcgatgccgcagagaaagctcatgcagatgcagtacaccctggttacggttttctttcagagaacgcagactttgctgcggcagtggaaaaagcaggaatgactttcataggtccctcctcagatgttatgaataagataaaggacaaacttgacggaaaaagagttgcaaaaatggctggagtacctatagctcctggctccgatggtccggttagttcgttagatgaagcactaaagattgcagaaaaaatcggttatccaataatggtaaaggctgcaagcggcggtggaggagtcgggataactagagtagacaatcctgaccaactagttgaagtgtgggaaagaaacaaaagattggcttatcaagctttcggaaaagcggacctttatatagaaaaatatgctgtaaatccgagacatatagaatttcagttaataggcgataaatatggagattacgtagtagcttgggaaagagaatgcacaatccaaagaagaaaccagaaattaatagaggaggcaccttctcctgcattaaaaatggaagaaagggaaaaaatgtttgaaccaataatgaagttcggtcaaataattcactactttacaatgggcacctttgaaactgcattttctgacgttactagggagttctatttcttagagttaaacaaaagattacaagtagagcatcctaccactgagttaatctttaggatagatttagttaagcttcaaatactccttgctgcaggagagcatttaccttttacacaagaagaacttaataagagagtaagaggtgcagcaatagaatatagaataaatgcagaagatcctttgaataatttcacgggcagttctggttatattacttattataaggagcctactggtccaggggtcagagtagatagcggagttgaagcaggaagctgggttccgcccttttacgattcccttatatctaaattaatagtatacggcgaaagtagggcctacgctattcaggctggaataagagctttgaatgactataagataggtggcgtaaagacgactattgagctttacaaatggatcatgagggatcctgactttcaagaaggaagattctctacatcgtatattgcacaaaaaactgaacaatttacaaagtatttgagacaacaagaggaattaagagcagcactagcattagaaatccagagtaggggactaaatagacaaggagcaactccagttacaacttcgcaaaggcaacccaaatctgcatggaagacttacggtttggtttctcaagcctcttcgagggtgatgtggtaa

SEQ ID NO: 72 - DNA, 1542 bp, Sulfolobus tokodaii
atgccaccttttggaaaagtactcgttgcaaatagaggagaaattgcagtaagagttatgaaagcaatcaaagaaatgggcatgaaagctgttgctgtgtattcagaagctgataaatatgctttgcacgtaaaatatgcagatgaggcttattatatagggccgccaccagcattagaaagctacttaaatattcaagccattattgatgcagcagaaaaagctcatgctgatgcagttcatcctggttacggtttcttatctgaaaacgcagattttgctgaagctgttgtaaaggctggattaacatggattggaccaccagtagatgcgatgagagctattaagagtaagctagacggcaaaagaattgcaaaacaagctggagtacctatttcacctggttctgacggtccagttgatagtttagatgaggctctaaaattagctgaaaaaattggctacccaataatggtaaaagcggcattcggtggtggaggtactggaataactagggttgataatccagaccagttagtcgaagtttgggaaaggaataaaagattagcttatcaagcctttggtaaagctgatttatatattgaaaaggcggcagttaatccaagacatattgagtttcagctaataggagataaatatggcaactatgtagtagcatgggaaagagagtgtacaattcaaagaagaaatcagaaattaattgaagaagcaccctcaccagctctaaaaatggaagaacgagaaagaatgtttgaaccaattatcaagttcggtcaaattattcattattacacattaggaacatttgaaacagttttctcagatactacgagagaattctatttccttgaattaaataaaagacttcaagttgaacacccaattaccgaaatgatatttagaattgacttagtaaaacttcaaataaatatcgctgcaggtgaacctttaccttttacccaagaagaattaaataaaagagtaagagggcacgcaatcgaatatagaatcaatgctgaagatccgttaaatgactttactggtagctcgggatttatcacatactataaggaaccgactggtccgggagtaagagtagatagtggtgttactttaggtagttacgtaccaccattttacgattcgttaatttcaaaactcattgtatatggcgaaaatagagcttatgccatacaggctggaataagagctttaaatgattataaaataggtggagttagaacaacaattgaactatacaaatggatatcacaagaggaagattttcaaaaaggtaaattctcaactgcatatatagctgagaagagagaacaatacctaaaatacttaaaagcaaaagagcagatgaaagccgctttagctgcaacgttatatcaaagaggattgttaaagaaagcaacgactacagttaattctactcaaccacaaaatcaaacaaagaggtcgaattggaaaacatacggtttattgcaacaatcctcttatagggtgatgtggtaa

SEQ ID NO: 73 - DNA, 1530 bp, Acidianus brierleyi
atgccgccttttagtagagttttagttgcgaacagaggagaaatagctaccagagtgcttaaggcgataaaagaaatgggaatgactgcaattgcagtatattctgaggcagataaatacgccgttcatacaaaatatgctgatgaagcttattatattggtaaggctcctgcgttagatagctatcttaatattgaacatataatagacgctgctgaaaaagctcatgttgacgcaattcatcctggatacggatttttatcagagaatgcagaattcgcagaagcagtagaaaaagctggcataacgtttatcggtccttcatccgaggtcatgagaaaaataaaggataaactagatggaaaaagattagcaaatatggcaggagttcctacagctcctggatcagacggccctgttacctctatagacgaagcgttaaagttagctgaaaaaataggatacccaataatggttaaggctgctagcggaggtggaggtgtaggtataacaagagtagataatcaggatcaattaatggacgtttgggaaagaaataaaagattagcttatcaagcctttggaaaagcagacttatttatagaaaaatacgctgtaaatcctaggcatatagaatttcaattaataggagataaatatggtaattatgtagtagcttgggaaagagaatgtactattcagaggagaaatcaaaaactaatagaggaagcgccatctcctgctcttaaaatggaagaaagagaatctatgtttgagccaatcataaaatttgggaaattgataaactattttacattaggtacatttgaaactgctttttcggacgtttctagagatttctactttttagaactcaataagagattacaagttgaacatcctaccacagagcttatatttaggatagatttggtaaaactgcagataaaacttgctgcaggagagcatctgccttttagccaagaggatctaaacaagagagttagaggaacagcaatagaatatagaataaatgcagaagacgctttaaataattttactggaagttccggatttgtaacatattatagggagcctacagggcctggcgttagagtagatagtggaatagaatctggtagctacgttcctccatattacgattccctagtatctaaattaatagtttatggggaaagtagagaatatgctatacaagccggaataagagcgttagctgactataaaataggaggaataaaaactactatagagctttataaatggataatgcaagatccagattttcaagaaggaaaattcagtacttcgtatatttcacaaaaaactgatcaatttgtaaaatatttgagagaacaagaggagataaaagcagccatagctgcagaaattcagagtagaggacttttgagaacaagcagcactgataacaaaggtaaagcccaaagtaagtctggttggaagacttacggtataataacgcaatcttctacgagggtgatgtggtaa

SEQ ID NO: 74 - DNA, 1533 bp, Metallosphaera sedula
atgccaccctttagtagagttttggttgcaaacaggggagaaattgcagtaagggtaatgaaggcaataaaggaaatgggaatgacagcaatagctgtttactctgaggctgacaagtacgcagtccacgttaagtatgccgatgaagcttattatattggaccctcgccggccttggaaagttacctcaacataccccacatcattgacgcagcggagaaggctcacgctgacgctgttcatcctggatatggattcttgtcggagaatgctgacttcgtggaggcagttgaaaaggcaggaatgacttacataggtccctctgctgaggtcatgagaaagataaaggataagctggatgggaaaaggatagcccagttatctggtgtccccattgcccctggctcggatggccccgtagaatccattgacgaggctcttaagttggctgagaagataggataccccatcatggttaaggccgctagcgggggtggtggagtaggtataacaaagatagatacacctgaccagctcattgacgcatgggaaagaaacaagaggttagctacacaagccttcggacgatctgatctatacatagaaaaagccgccgtaaaccctaggcacattgagtttcagttaattggcgataagtacggcaactatgtcgttgcttgggagagggaatgtactattcagagaagaaaccagaagttgatagaggaggcaccatctccagcaatcacaatggaagaaaggtcacgaatgttcgagcctatatacaaatatgggaagttaattaattactttaccctgggtactttcgagacagttttctctgatgccacaagggagttctacttccttgagctgaacaaaaggcttcaggtagaacacccagttactgagttaatattcagaattgatctggtaaagctacagataaggctagctgcaggagaacatttgccattcacgcaggaggaactcaacaagagggcgagaggtgcagcaatagagttcaggataaatgccgaggatccaataaataatttcagcggaagctcaggtttcattacgtactacagggagcccacgggtcctggagtgagaatggatagcggtgtaacggagggaagctgggtacctcctttctacgactctctagtatcgaagttgattgtgtatggagaagacaggcaatacgcaatacaaactgccatgagggcactagacgattacaagattggcggagtcaaaacgactataccgctatacaagctcatcatgagggatcccgactttcaggaaggaaggttcagtactgcctatatttcccagaagattgactcaatggttaagaaactgaaggccgaagaggagatgatggcttcagtggccgcagttcttcagagcaggggactccttagaaagaaggcttcagctcctcaggagcaggcgaaaccaggctcaggatggaagagttacggtatcatgatgcagagcactcctagggtgatgtggggatga

SEQ ID NO: 75 - DNA, 1533 bp, Metallosphaera cuprina
atgccaccatttagtagagtacttgtctccaataggggcgaaatagctgtaagagtaatgaaagcgataaaggaaatgggaatgaccgcgatagctgtatattctgaggctgataaatatgcactccatgtaaaatatgcggacgaagcttattatattggtccgtctccggcactggaaagttacctgaacataccacgtattatagacgcggcagagaaggctcatgctgacgctatccatccaggatacggtttcttatccgaaaatgcagattttgtagaggcagtagaaaaggccggaattacgtacataggtccttcagcagacgttatgagaaagataaaagacaaattagatggaaagagaatagctgttcaggcaggagttcctattgcccctggctcagatggtccagtgagctctatagacgaggcattgaaattggcagaaaggataggttatccgataatggttaaggcggcaagtggaggaggaggtgtagggataactaaaatagattctccagatcaattaatcgatgcatgggaaagaaataaaagattggcaacgcaggcctttggaaagtctgacctttatattgaaaaagctgcagttaatccaagacatatagagtttcagctaataggagataagtatggtaactacgtagtagcatgggaaagggagtgtacaatccagagaagaaatcagaaattgatagaggaagccccatcacctgccattacaatggaagaaaggtcaaagatgttcgagcctataatgaaatatggccatatacttaattattttacattaggtacattcgaaacagtgttttctgacgctactagggaattctatttcctagagctaaacaaaagattacaggtcgagcatcccacaacggagctaatattcagaatggacctagttaagctccagataagactggctgctggagaacatcttccatttacacaggaagagttaaacaagagagctagaggcgcttcaatagaattcagaatcaacgcagaggatcctttgaacgattttagtggaagctctgggtacataacgtactataaggaaccctctggtccaggagttaggacggacagcggtgtagttgaagggagctgggtaccacctttttatgactcattaatttcaaaactaatagtttacggcgagaacagaccttatgcgatacaaaccgcaataagggctttagacgattataagatagggggagttaaaaccactatctctttatacaagctgataatgagggatccggactttcaggagggtaagtttagtactgcttacatatcacagaaaatgaattcgttaactaagaaacttagaacagaggaggagatgttagcctccattgccgtagttttacaaagtagaggcatgttaagaaagagggctcctgtaagtcaggtacagacgaagtctgaatcgggctggaaaagttacggactgatcatgcagagctcccctagggtgatgtggagatga

SEQ ID NO: 76 - DNA, 1533 bp, Sulfolobus acidocaldarius
atgcccccgttcggaaaagtcttggtcgcaaataggggagaaatagcaataagggtaatgaaagccgtaaaggaaatgggaatgaaagcagttgcagtgtactcagaggctgacaagaactctcttcatgtcaagtacgctgatgaagcctactatataggaccttctccggcaattcaaagctacttgaatatagagtcaattatcagcgtagctgagaaagcacatgttgatgcagttcatccgggttatgggttcctgtctgaaagggcagatttcgcagaagcagtggaaaaggcaggggtggtgttcataggaccttctccccatgcaatgaactccattaaaagtaaacttgacggaaagagacttgcaaaggcttctggagttcccatatcacctggttctgatggaccagtggagaatttagatgaggctattaagatagcagataggataggctaccctataatggtgaaagcagcctatggaggaggaggcacaggtataactaaggtagattctcaagagcaacttattgaggtttgggagagaaataagagattggcataccaggcatttggaaaagctgacttatatatagagaaagccgcagtaaatcctagacacatagagtttcaactcataggagataaatatggcaattatgtggttgcatgggagagagaatgcacgatacagaggagaaatcaaaaactaattgaagaagccccctcacctgtagttaagatggaagagagagaaagaatgtttgaacctataatgaaatttgggcaactaataagatatcacaccttaggtacattcgagacagtgttctccgatgtaagcagagagttttacttccttgagttaaacaagaggttacaagttgaacatccaataacagagaccatattcagaatagatctagtgaaattacagataagactagctgcagatgaacatttacccttcacacaagaggagttgaataagagggtaagaggacacgcaatagaatatagaattaactcagaggatccaatgagtgatttttcgggaagttcaggaactattacctattacgaggagccatcaggacctggagttagagtagacagtggaatcaccttaggcagttatgttccacctttctacgattcattaatagctaagttgatagtttacggtgaagataggatctcagcactacagtcagggcaaagggctctgagcgatttcaaaattggtggtgtaaaaacgacaatagaattatataaatggataacgagagaagaagattttgtaaatgccaaatttactaccgcatacataagtcagaaaagcaaggagtttttagaatacctgaagagaaaagagatgactaaagctgttatagcatcagtcatgtacagtaaaggatatgttaaaaagagtggtaacggcaaaaaggagacagtttcaaacaataaaaacaaatggaagacttatggaataatgtcacaatcttcatatagggtgttgtggtaa

SEQ ID NO: 77 - DNA, 1533 bp, Sulfolobus islandicus
atgccaccctttaataaagttctcgtagccaacagaggagaaattgccataagggttatgaaagccgtaaaagaaatgggaatgaaggcagttgctgtatattctgatgctgataaatatgccccccacgttaaatatgcagatgaagcttattggatagggccacctccagctttagaaagttacttgaatattgaaagaattatcgatgcggcagaaaaggctcatgcagatgccatacatcccggttatgggttcctttccgaaaacgcgccttttgtagaagctgttgaaaaagctggaatggtgttcataggaccttctgcttctgtaatgaatagaatcaaggacaaattggaaggaaagatgatagcaagaaaggctggtgtccctacgtctcctggacctttaacaccaattgaaaatgtggatgaagcgttaaagatagctgaggaaataggatatcccataatgttgaaggctgcgggaggtggagccggagtgggtattataaaggttgataatcctagtgaactagctgaggcttttgaaagaagcaaaagattagcgtactctgcctttggcagggcagaaatttacatagaaaaggctgctataaaaccaaaacacattgaaacgcaattaataggagataagtatggaaattacgtagtcgcctttgaaagagaatgcacaattcaaagaaggaaccagaagttaattgaagaagccccctctccatccattaaagaagaggaaagaaaggagattattgaggcgtcaataagatttgggaaagaaattaactactttaccttaggtaccatggagttcgtgttctcaccagttactcgtgaattttacttcttagaaattaataaaagagttcaagtagagcacacagtaactgagttcataactggaatagacttagtcaaattacagataagactagctgctggtgaatatttacctttttctcaagaagacttgaaaataagaggacatgcaatacagtttagaattaacgctgaggacccattaaataacttcacgcctcaatccggatacataacctattataaggaaccaactggtcccggtgtgagagtggatagtggtatagaacttggttcatgggttccaccatattatgatccccttgtttccaagttaattgtttatggacaaagtagggactacgcgattcaagtcggactaagggctctaaacgattataaaataggaggagttaaaactacaattccattatataagttgatcttgcaagatccagacttctgggaaggtaatttcactactgcatatatatctgagaaagcagagtactttacaaccaaattaaaagaagaacaagagatacaaattgcaatagccatttcgatttataataggggattgatgaagaagaaaaaaactgaacagaaattacccatctcaactagtaatggtaaaagtaattggaaaacttatggtatcatattccaatcctctccaagggtgttgtggtaa

SEQ ID NO: 78 - DNA, 1533 bp, Sulfolobus islandicus
atgccaccctttaataaagttctcgtagccaacagaggagaaattgccataagggttatgaaagccgtaaaagaaatgggaatgaaggcagttgctgtatattctgatgctgataaatatgccccccacgttaaatatgcagatgaagcttattggatagggccacctccagctttagaaagttacttgaatattgaaagaattatcgatgcggcagaaaaggctcatgcagatgctgtacatcccggttatgggttcctttccgaaaacgcgtcttttgtagaagctgttgaaaaagctggaatggttttcataggaccttctgcttctgtaatgaatagaatcaaggacaaattggaaggaaagatgatagctagaaaggctggtgtccccacgtcccctggacctttaacaccaattgaaaatgtagatgaggcgttaaagatagctggagaaataggatatcccataatgttgaaggctgcgggaggtggagcaggagtgggtattataaaggttgataatcctagtgaactagctgaggcttttgaaagaagcaaaagattagcgtactctgcctttggcagggcagaaatttacatagaaaaggctgctataaaaccaaaacacattgaaacgcaattgataggagataagtatggaaattacgtagtcgcctttgaaagagaatgcacaattcaaagaaggaaccaaaagttaatcgaagaagccccctctccatccattaaagaagaggaaagaaaggagattattgaggcgtcaataagatttgggaaagaaattaactactttaccttaggtaccatggagttcgtgttctcaccagttactcgtgaattttacttcttagaaattaataaaagagttcaagtagagcacacagtcactgagttcataactggaatagacttagtcaaattacagataagactagctgctggtgaatatttacctttttctcaagaagacttgaaaataagaggacatgcaatacagtttagaattaacgctgaggacccattaaataacttcacgcctcaatccggatacataacctattataaggaaccaactggtcccggtgtgagagtggatagcggtatagaacttggttcatgggttccaccatattatgatccccttgtttccaagttaattgtttatggacaaagtagagactacgcgattcaagtcggactaagggctctaaacgattataaaataggaggagttaaaactacaattccattatataagttgatcttgcaagatccagacttctgggaaggtaatttcactactgcatatatatctgagaaagcagagtactttacgaccaaattaaaagaagaacaagagatacaaattgcaatagccatttcgatttataataggggattgatgaagaagaaaaaaactgaacagaaagtacccatctcaactagtaatggtaaaagtaattggaaaacttatggtatcatattccaatcctctccaagggtgttgtggtaa

SEQ ID NO: 79 - DNA, 1521 bp, Sulfolobus solfataricus
atgccaccctttaataaagtgctcgtagccaatagaggggaaattgctataagagtcatgaaagctgtaaaggaaatgggaatgaaggcagttggtgtatactccgatgctgataaatatgccctacacgtgaaatatgcagatgaagcctattggataggacctccaccagctttagaaagctatttgaatatcgagagaataattgatgcagcagaaaaagctcacgtagatgctatacatccaggttacggattcctttctgaaaatgcgtcttttgtagaagcagttgaaaaagctggaataacgttcatagggccttctgctagtgtaatgaataaaataaaggataagttagagggaaagatgatagccaaaaaggctggcgttcccatatcgcctggacctttgacaccagtcgacaacgtgaatgaagcgttaaagatcgctgaggagataggatatcccataatgctaaaggctgcaggaggcggagcgggggtaggtattataaaggttgataatcctagcgaactagctgaggcctttgaaagaagcaaaagactagcttactcagccttcggcagggccgaaatctacatagaaaaagctgctataaaaccaaaacatattgaaacgcaattgataggagataagtatggaaattatgtggtagcttttgaaagagagtgcacgattcaaagaagaaatcaaaagttaattgaagaagccccttccccatccattaaagaagaggaaagaaaagagattattgaggcatcgattagatttgggaaagaaattaactactttactttaggtaccatggagtttgtgttctcgccagttacccgtgaattttatttcttagaattaaataaaagagttcaagtagagcatacagtcactgaattcataactggaatagacttagttaaattacagataagattggcatctggagaatatttacctttctctcaagaggatttgaaaataaggggacatgcaatacagtttagaattaacgctgaggatcctttaaacaatttcacaccccaatctggatacataacctattatagagaaccaactggccctggtgtgagagtagatagtggtatagaatctggctcatgggttcctccttattatgatccccttgtttctaagctgattgtttatggacaaaatagagactatgcaattcaagtcggattgagggctttaaacgattataaaataggaggagttaaaactacgattccattgtataagttgatcttgcaagatcctgacttttgggaaggtaactttactactgcctacatatccgagaaaatggagtactttacgaccaaattaaaagaagaacaagagatgcaaatcgcaatggctatttccattttcaataggggattaatcaagagaaagaaaactgaacaaaaggtgttaactagtaaaagtaattggaaaacttatggtattatatcccaatcctctcccaaggtgttatggtga

SEQ ID NO: 80 - DNA, 1533 bp, Sulfolobus islandicus
atgccaccctttaataaagttctcgtagccaacagaggagaaattgccataagggttatgaaagccgtaaaagaaatgggaatgaaggcagttgctgtatattctgatgctgataaatatgccccccacgttaaatatgcagatgaagcttattggatagggccacctccagctttagaaagttacttgaatattgaaagaattatcgatgcggcagaaaaggctcatgcagatgctgtacatcccggttatgggttcctttccgaaaacgcgtcttttgtagaagctgttgaaaaagctggaatggttttcataggaccttctgcttctgtaatgaatagaatcaaggacaaattggaaggaaagatgatagctagaaaggctggtgtccccacgtcccctggacctttaacaccaattgaaaatgtagatgaggcgttaaagatagctggagaaataggatatcccataatgttgaaggctgcgggaggtggagcaggagtgggtattataaaggttgataatcctagtgaactagctgaggcttttgaaagaagcaaaagattagcgtactctgcctttggcagggcagaaatttacatagaaaaggctgctataaaaccaaaacacattgaaacgcaattgataggagataagtatggaaattacgtagtcgcctttgaaagagaatgcacaattcaaagaaggaaccaaaagttaatcgaagaagccccctctccatccattaaagaagaggaaagaaaggagattattgaggcgtcaataagatttgggaaagaaattaactactttaccttaggtaccatggagttcgtgttctcaccagttactcgtgaattttacttcttagaaattaataaaagagttcaagtagagcacacagtcactgagttcataactggaatagacttagtcaaattacagataagactagctgctggtgaatatttgcctttttctcaagaagacttgaaaataagaggacatgcaatacagtttagaattaacgctgaggacccattaaataacttcacgcctcaatccggatacataacctattataaggaaccaactggtcccggtgtgagagtggatagcggtatagaacttggttcatgggttccaccatattatgatccccttgtttccaagttaattgtttatggacaaagtagagactacgcgattcaagtcggactaagggctctaaacgattataaaataggaggagttaaaactacaattccattatataagttgatcttgcaagatccagacttctgggaaggtaatttcactactgcatatatatctgagaaagcagagtactttacgaccaaattaaaagaagaacaagagatacaaattgcaatagccatttcgatttataataggggattgatgaagaagaaaaaaactgaagagaaagtacccatctcaactagtaatggtaaaagtaattggaaaacttatggtatcatattccaatcctctccaagggtgttgtggtaa

SEQ ID NO: 81 - DNA, 1533 bp, sulfolobus islandicus
atgccaccctttaataaagttctcgtagccaacagaggagaaattgccataagggttatgaaagccgtaaaagaaatgggaatgaaggcagttgctgtatattctgatgctgataaatatgccccccacgttaaatatgcagatgaagcttattggatagggccacctccagctttagaaagttacttgaatattgaaagaattatcgatgcggcagaaaaggctcatgcagatgctgtacatcccggttatgggttcctttccgaaaacgcgccttttgtagaagctgttgaaaaagctggaatggtgttcataggaccttctgcttctgtaatgaatagaatcaaggacaaattggaaggaaagatgatagcaagaaaggctggtgtccctacgtcccctggacctttaacaccaattgaaaatgtggatgaagcgttaaagatagctggggaaataggatatcccataatgttgaaggctgcgggaggtggagccggagtgggtattataaaggttgataatcctagtgaactagctgaggcttttgaaagaagcaaaagattagcgtactctgcctttggcagggcagaaatttacatagaaaaggctgctataaaaccaaaacacattgaaacgcaattaataggagataagtatggaaattacgtagtcgcctttgaaagagaatgcacaattcaaagaaggaaccagaagttaattgaagaagccccctctccatccattaaagaagaggaaagaaaggagattattgaggcgtcaataagatttgggaaagaaattaactactttaccttaggtaccatggagttcgtgttctcaccagttactcgtgaattttacttcttagaaattaataaaagagttcaagtagagcacacagtaactgagttcataactggaatagacttagtcaaattacagataagactagctgctggtgaatatttacctttttctcaagaagacttgaaaataagaggacatgcaatacagtttagaattaacgctgaggacccattaaataacttcacgcctcaatccggatacataacctattataaggaaccaactggtcccggtgtgagagtggatagcggtatagaacttggttcatgggttccaccatattatgatccccttgtttccaagttaattgtttatggacaaagtagagactacgcgattcaagtcggactaagggctctaaacgattataaaataggaggagttaaaactacaattccattatataagttgatcttgcaagatccagacttctgggaaggtaatttcactactgcatatatatctgagaaagcagagtactttacgaccaaattaaaagaagaacaagagatacaaattgcaatagccatttcgatttataataggggattgatgaagaaggaaaaaactgaacagaaagtacccatctcaactagtaatggtaaaagtaattggaaaacttatggtatcatattccaatcctctccaagggtgttgtggtaa

SEQ ID NO: 82 - DNA, 1533 bp, Sulfolobus islandicus
atgccaccctttaataaagttctcgtagccaacagaggagaaattgccataagggttatgaaagccgtaaaagaaatgggaatgaaggcagttgccgtatattctgatgctgataaatatgccccccacgttaaatatgcagatgaagcttattggatagggccacctccagctttagaaagttacttgaatattgaaagaattatcgatgctgcagaaaaggctcatgcagatgctgtacatcccggttatgggttcctttccgaaaacgcgccttttgtagaagctgttgaaaaagctggaatggtgttcataggaccttctgcttctgtaatgaatagaatcaaggacaaattggaaggaaagatgatagcaagaaaggctggtgtccctacgtcccctggacctttaacaccaattgaaaatgtggatgaggcgttaaagatagctggggaaataggatatcccataatgttgaaggctgcgggaggtggagccggagtgggtattataaaggttgataatcctagtgaactagctgaggcttttgaaagaagcaaaagattagcgtactctgcctttggtagggcagaaatttacatagaaaaggctgctataaaaccaaaacacattgaaacgcaattaataggagataagtatggaaattacgtagtcgcctttgaaagagaatgcacgattcaaagaaggaaccagaagttaattgaagaagccccctctccatccattaaagaagaggaaagaaaggagattattgaggcgtcaataagatttgggaaagaaattaactactttaccttaggtaccatggagttcgttttctcaccagttactcgtgaattttacttcttagaaattaataaaagagttcaagtagagcacacagtaactgagttcataactggaatagacttagtcaaattacagataagactagctgctggtgaatatttacctttttctcaagaagacttgaaaataagaggacatgcaatacagtttagaattaacgctgaggacccattaaataacttcacgcctcaatccggatacataacctattataaggaaccaactggccccggtgtgagagtggatagcggtatagaacttggttcatgggttccaccatattatgatccccttgtttccaagttaattgtttatggacaaagtagagactacgcgattcaagtcggactaagggctctaaacgattataaaataggaggagttaaaactacaattccattatataagttgatcttgcaagatccagacttctgggaaggtaatttcactactgcatatatatctgagaaagcagagtactttacgaccaaattaaaagaagaacaagagatacaaattgcaatagccatttcgatttataataggggattgatgaagaagaaaaaaactgaacagaaattacccatctcaactagtaatggtaaaagtaattggaaaacttatggtatcatattccaatcctctccaagggtgttgtggtaa

SEQ ID NO: 83 - DNA, 1533 bp, Sulfolobus islandicus
atgccaccctttaataaagttctcgtagccaacagaggagaaattgccataagggttatgaaagccgtaaaagaaatgggaatgaaggcagttgctgtatattctgatgctgataaatattccccccacgttaaatatgcagatgaagcttattggatagggccacctccagctttagaaagttacttgaatattgaaagaattatcgatgcggcagaaaaggctcatgcagatgctgtacatcccggttatgggttcctttccgaaaacgcgtcttttgtagaagctgttgaaaaagctggaatggttttcataggaccttctgcttctgtaatgaatagaatcaaggacaaattggaaggaaagatgatagctagaaaggctggtgtccccacgtcccctggacctttaacaccaattgaaaatgtagatgaggcgttaaagatagctggagaaataggatatcccataatgttgaaggctgcgggaggtggagcaggagtgggtattataaaggttgataatcctagtgaactagctgaggcttttgaaagaagcaaaagattagcgtactctgcctttggcagggcagaaatttacatagaaaaggctgctataaaaccaaaacacattgaaacgcaattgataggagataagtatggaaattacgtagtctcctttgaaagagaatgcacaattcaaagaaggaaccaaaagttaatcgaagaagccccctctccatccattaaagaagaggaaagaaaggagattattgaggcgtcaataagatttgggaaagaaattaactactttaccttaggtaccatggagttcgtgttctcaccagttactcgtgaattttacttcttagaaattaataaaagagttcaagtagagcacacagtcactgagttcataactggaatagacttagtcaaattacagataagactagctgctggtgaatatttacctttttctcaagaagacttgaaaataagaggacatgcaatacagtttagaattaacgctgaggacccattaaataacttcacgcctcaatccggatacataacctattataaggaaccaactggtcccggtgtgagagtggatagcggtatagaacttggttcatgggttccaccatattatgatccccttgtttccaagttaattgtttatggacaaagtagagactacgcgattcaagtcggactaagggctctaaacgattataaaataggaggagttaaaactacaattccattatataagttgatcttgcaagatccagacttctgggaaggtaatttcactactgcatatatatctgagaaagcagagtactttacgaccaaattaaaagaagaacaagagatacaaattgcaatagccatttcgatttataataggggattgatgaagaagaaaaaaactgaacagaaagtacccatctcaactagtaatggtaaaagtaattggaaaacttatggtatcatattccaatcctctccaagggtgttgtggtaa

SEQ ID NO: 84 - DNA, 1533 bp, Sulfolobus solfataricus
atgtcaccctttaataaagttctcgtagccaacagaggagaaattgccataagggttatgaaagccgtaaaagaaatgggaatgaaggcagttgctgtatattctgatgctgataaatatgccccccacgttaaatatgcagatgaagcttattggatagggccacctccagctttagaaagttacttgaatattgaaagaattatcgatgcggcagaaaaggctcatgcagatgctgtacatcccggttatgggttcctttccgaaaacgcgtcttttgtagaagctgttgaaaaagctggaatggttttcataggaccttctgcttctgtaatgaatagaatcaaggacaaattggaaggaaagatgatagctagaaaggctggtgtccccacgtcccctggacctttaacaccaattgaaaatgtagatgaggcgttaaagatagctggagaaataggatatcccataatgttgaaggctgcgggaggtggagcaggagtgggtattataaaggttgataatcctagtgaactagctgaggcttttgaaagaagcaaaagattagcgtactctgcctttggcagggcagaaatttacatagaaaaggctgctataaaaccaaaacacattgaaacgcaattgataggagataagtatggaaattacgtagtcgcctttgaaagagaatgcacaattcaaagaaggaaccaaaagttaatcgaagaagccccctctccatccattaaagaagaggaaagaaaggagattattgaggcgtcaataagatttgggaaagaaattaactactttaccttaggtaccatggagttcgtgttctcaccagttactcgtgaattttacttcttagaaattaataaaagagttcaagtagagcacacagtcactgagttcataactggaatagacttagtcaaattacagataagactagctgctggtgaatatttacctttttctcaagaagacttgaaaataagaggacatgcaatacagtttagaattaacgctgaggacccattaaataacttcacgcctcaatccggatacataacctattataaggaaccaactggtcccggtgtgagagtggatagcggtatagaacttggttcatgggttccaccatattatgatccccttgtttccaagttaattgtttatggacaaagtagagactacgcgattcaagtcggactaagggctctaaacgattataaaataggaggagttaaaactacaattccattatataagttgatcttgcaagatccagacttctgggaaggtaatttcactactgcatatatatctgagaaagcagagtactttacgaccaaattaaaagaagaacaagagatacaaattgcaatagccatttcgatttataataggggattgatgaagaagaaaaaaactgaacagaaagtacccatctcaactagtaatggtaaaagtaattggaaaacttatggtatcatattccaatcctctccaagggtgttgtggtaa

SEQ ID NO: 85 - DNA, 1533 bp, Sulfolobus islandicus
atgccaccctttaataaagttctcgtagccaacagaggagaaattgccataagggttatgaaagccgtaaaagaaatgggaatgaaggcagttgctgtatattctgatgctgataaatatgccccccacgttaaatatgcagatgaagcttattggatagggccacctccagctttagaaagttacttgaatattgaaagaattatcgatgcggcagaaaaggctcatgcagatgctgtacatcccggttatgggttcctttccgaaaacgcgtcttttgtagaagctgttgaaaaagctggaatggttttcataggaccttctgcttctgtaatgaatagaatcaaggacaaattggaaggaaagatgatagctagaaaggctggtgtccccacgtcccctggacctttaacaccaattgaaaatgtagatgaggcgttaaagatagctggagaaataggatatcccataatgttgaaggctgcgggaggtggagcaggagtgggtattataaaggttgataatcctagtgaactagctgaggcttttgaaagaagcaaaagattagcgtactctgcctttggcagtgcagaaatttacatagaaaaggctgctataaaaccaaaacacattgaaacgcaattgataggagataagtatggaaattacgtagtcgcctttgaaagagaatgcacaattcaaagaaggaaccaaaagttaatcgaagaagccccctctccatccattaaagaagaggaaagaaaggagattattgaggcgtcaataagatttgggaaagaaattaactactttaccttaggtaccatggagttcgtgttctcaccagttactcgtgaattttacttcttagaaattaataaaagagttcaagtagagcacacagtcactgagttcataactggaatagacttagtcaaattacagataagactagctgctggtgaatatttacctttttctcaagaagacttgaaaaaaagaggacatgcaatacagtttagaattaacgctgaggacccattaaataacttcacgcctcaatccggatacataacctattataaggaaccaactggtcccggtgtgagagtggatagcggtatagaacttggttcatgggttccaccatattatgatccccttgtttccaagttaattgtttatggacaaagtagagactacgcgattcgagtcggactaagggctctaaacgattataaaataggaggagttaaaactacaattccattatataagttgatcttgcaagatccagacttctgggaaggtaatttcactactgcatatatatctgagaaagcagagtactttatgaccaaattaaaagaagaacaagagatacaaattgcaatagccatttcgatttataataggggattgatgaagaagaaaaaaactgaacagaaagtacccatctcaactagtaatggtaaaagtaattggaaaacttatggtatcatattccaatcctctccaagggtgttgtggtaa

SEQ ID NO: 86 - DNA, 1533 bp, Sulfolobus islandicus
atgccaccctttaataaagttctcgtagccaacagaggagaaattgccataagggttatgaaagccgtaaaagaaatgggaatgaaggcagttgccgtatattctgatgctgataaatatgccccccacgttaaatatgcagatgaagcttattggatagggccacctccagctttagaaagttacttgaatattgaaagaattatcgatgctgcagaaaaggctcatgcagatgctgtacatcccggttatgggttcctttccgaaaacgcgccttttgtagaagctgttgaaaaagctggaatggtgttcataggaccttctgcttctgtaatgaatagaatcaaggacaaattggaaggaaagatgatagcaagaaaggctggtgtccctacgtcccctggacctttaacaccaattgaaaatgtggatgaggcgttaaagatagctggggaaataggatatcccataatgttgaaggctgcgggaggtggagccggagtgggtattataaaggttgataatcctagtgaactagctgaggcttttgaaagaagcaaaagattagcgtactctgcctttggcagggcagaaatttacatagaaaaggctgctataaaaccaaaacacattgaaacgcaattaataggagataagtatggaaattacgtagtcgcctttgaaagagaatgcacgattcaaagaaggaaccagaagttaattgaagaagccccctctccatccattaaagaagaggaaagaaaggagattattgaggcgtcaataagatttgggaaagaaattaactactttaccttaggtaccatggagttcgttttctcaccagttactcgtgaattttacttcttagaaattaataaaagagttcaagtagagcacacagtaactgagttcataactggaatagacttagtcaaattacagataagactagctgctggtgaatatttacatttttctcaagaagacttgaaaataagaggacatgcaatacagtttagaattaacgctgaggacccattaaataacttcacgcctcaatccggatacataacctattataaggaaccaactggccccggtgtgagagtggatagcggtatagaacttggttcatgggttccaccatattatgatccccttgtttccaagttaattgtttatggacaaagtagagactacgcgattcaagtcggactaagggctctaaacgattataaaataggaggagttaaaactacaattccattatataagttgatcttgcaagatccagacttctgggaaggtaatttcactactgcatatatatctgagaaagcagagtactttacgaccaaattaaaagaagaacaagagatacaaattgcaatagccatttcgatttataataggggattgatgaagaagaaaaaaactgaacagaaattacccatctcaactagtaatggtaaaagtaattggaaaacttatggtatcatattccaatcctctccaagggtgttgtggtaa

SEQ ID NO: 87 - DNA, 1368 bp, Chloroflexus aggregans
atgattcgcaaagttcttgtagcaaaccgcggcgaaattgccgtgcgtatcattcgggcctgccaagagctgggtattcggacggtcgctgcctatagcactgccgaccgtgattcgctcgctgttcgtttagccgacgaagcggtctgtatcgggccgcccccaccggcaaagtcgtacctcaatgcgcccgcactgatcagcgctgccctgatcaccggctgtgatgcagtccatccgggctacggcttcctctcggaaaatccctattttgccgagatgtgcgccgattgtaatctgatcttcgtcggcccaccacctgaaccgatccgcctgatgggggataaggcgattggacgcgaaaccatgcgtaaagccggtgtgccgaccgtacccggatcggacggtgaggtacgctcgctcgatgaggcaatcgatattgcgcgccagatcggctaccccgtcctgctgaaaccatccggtggtggcggtggacgtggcatgcgcgtcgcgtatgatgaagccgatctccaacgtgccttcgcaaccgcccgcgccgaggccgaagcagccttcggcaatggtgcgctcctcttggaaaagtatctgacccgtgtccgtcacgtcgaaattcaggtgctcgccgataagtatggtcacgccattcaccttggtgagcgcgattgctccgcgcaacgtcgtcaccagaagattgtcgaagaagcaccgtcaccggttgtgacccctgaattgcgggcgcgaatgggcgccgatgcggtgcgtgggattacgtcgatcgggtatgtcaacgccggtaccctcgagtttctcctcgatgaagagggtaactattacttcatcgaaatgaatacgcgcattcaggttgaacatccggtaaccgaacaggtgaccggcgttgatttggtgcgttggcagttgctgatcgccagcggtgaacgcttgacgctgcgccaagaagacattacaatagcgcggcacgctatcgaatgccggattaacgccgaggatccggagcgtgacttcttgccggcaagtggcgaggtggagttctatctcccacccggcggtcctggagtgcgggttgactcgcacctttactcaggatatatgccacccggtaactacgattcgttgttggcgaaaattattacttatggtgatacacgtgatgaggcgctcaatcgtatgcggcgagcgctgaatgaatgcgtgattaccggtatcaaaacaaccatcccgttccaattggcgttgatcgacgaccctgaatttcgcgccggaaagatccataccggctatgtcgccgaattgctacgccaatggaaagagtcgctcagcccggcgtga

SEQ ID NO: 88 - DNA, 1374 bp, Oscillochloris trichoides
atgctaaacaaggtacttatcgccaaccgtggcgaaatcgccgtccgcatcgtccgcgcctgccaagagctgggcatccgcaccgtggccgcctttagcgaagccgaccgcgactcgttggcggtacgcctcgccgatgaggcggtctgtattggccccgccgcaccggccaaatcctacctcaacaccccggccctgatcagcgccgcgctgatcaccggctgtgatgggattcatccgggctacggcttcctctccgagaatccctactttgccgagatctgtgccgagtgcaaactgaccttcatcggccccagtgccgagacgatccgcctgatgggggacaagtccatcggccgccaaacgatgcgcgccgccggagtgccgatcatccccggttccgagggtgaactgcaatcggtggaagaggcggtggatctggcccgtcagatcggctacccggtgctgctcaagccgagtgcgggtggtggtgggcgcggtatgcgcgtcgccaacgatgagagcgagctgattcgcgccttctcgaccgcccgcgccgaagccgagggggcctttgggcgtggcgatctgatcctcgaaaagtacttgcccaaggtgcgccacgtcgagatccaggtcttagccgatgcctatggccatgccatccacctcggcgaacgcgactgctcggcccagcgccgccaccagaagatcctcgaagaagccccctcgcccgtcgtcaccccagaggtgcgggcgcgcatgggtgccgacgccctgcgcggcattcagtccatcggctacctcaatgccgggacgcttgaatttctgatggaccccgatggcaactactacttcatcgagatgaacacgcgtattcaggtcgaacacccggtgactgaactcgtcaccgacactgatctgctgcgctggcagttgcggatcgcctctggcgaacgcttgacgctccaacagaatgatattagaatagctcggcatgcggttgaatgccgaatcaacgccgaagacccagagcgggattttctccctgctgggggcgagatcgaattctacctaccgcccggtgggccaggcgtgcgagtcgactcgcacctctatgcgggctacaacccacccggaagttacgactcactgttggcaaagatcatcacctttggcgatacccgcgacgatgcactgaaccgcatgcgccgcgcactccacgagtgcatcatcaccggggtgaagacgacgatcccgtttcagctacacctgatcgatgacccggccttccgcgccgggaacatctcgactggctatgttgcagaactgctccagcgctggaaggatgagcgggccgcaggcgtcgcctag

SEQ ID NO: 89 - DNA, 1365 bp, Roseiflexus sp
atgttcaacaaagtgctcatcgccaaccgtggcgaaattgcggttcgtatcgttcgcgcctgtcacgaacttggcgtgcgagccgttgtagcgtacagcgaagccgacaaatattcgcttgctgtgcgcctggctgacgaagccgtctgtatcggtcccgctgcatcggcgcgttcctacctcaatccatcggcgctcatcagcgcagcgttgatgaccgggtgcgaagcgatccatcccggctacggctttctttccgaaaatccgtacttcgctgaaatctgcgcggagtacaaactcacgtttatcggacctgacgcccacgccatccggatgatgggagacaaggcgctgggcaggaaaaccatgcgcgatgcaggggtcccaaccgtgccgggatcacgcggcgaactgcgcaccctcgaagaagcggtcgaggttgcgcatcagatcggctacccggtgctgctcaaaccatcgggcggcggcggcgggcgcgggatgcgcgttgcgcagaatgaacaggagttgatcaaagcctacccgacatcaaaagccgaggcggaggcggcgttcggcaatggcgccctgctgatggaaaaatacctgccgcaggtgcgccacgtcgaaattcaggtgctggcagaccggtacggtcacgcgattcacctcggcgaacgcgattgctcgtcgcagcgccgccaccagaagattgtcgaagaggcgccttcgcccgccgtgacgcccgaattgcgggagcgcatgggcgaagcagcgctcaaaggcgtccgtgccatcaattatgtcaatgcaggaaccatggagttcctgctcgatccggacggcaacttctacttcatcgagatgaatacacgcatccaggtcgagcatccggtcaccgaaatggtcaccggcatcgacctggtcaaatggcagttgcgcattgcggctggcgaaccgttgacgatcaagcagtcggacgtggtgatgcgcgggcacgctatcgaagcgcgcatcaacgctgaagaccctgaccgcgactttatgccgtcgggtggagagatcgagtactacctgccgcccggcggaccgggggtgcgtgtcgattcgcacctgtacgccgggtatgctccacccggctattacgactcgctcctggcgaaactgatcgtgtggggcgccgatcggaacgaagcgctcgttcgccttgaacgcgcgctgcgtgaatttgtgattaccggcattcataccaccattccattcacgctggcgatgctcgaagatccaccgttccgcgaaggccgaatctcgacgcggtacgttccagatctggtacagcgtatcaaggagagcaaactggagtag

SEQ ID NO: 90 - DNA, 1365 bp, Roseiflexus castenholzii
atgttcaacaaagtgctcatcgccaaccgtggcgaaattgccgtccgcatcgtccgcgcctgtcacgaacttggcgtgcgagcagtcgtggcatacagcgaagctgacaaatactcgctggctgtgcgcctggcggacgaagccgtgtgtattggtcccgctgcatcggcgcgatcctatctcaacccttcggcactgatcagcgcagcgctgatgaccggctgcgaagccattcatcctggctatggcttcctgtcggagaatccgtactttgccgagatctgcgccgagtacaaactgaagttcatcggtcccgacgccaatgccatccgaatgatgggagacaaggcgcttggcaggaagaccatgcgtgacgctggtgtgccaaccgtgccgggatcgcgcggcgaattgcgcaccctcgaagaggcggtcgaaacggcgcatcagatcggctacccggtgctcctcaagccttctggcggcggcggcgggcgcggcatgcgcgtcgcccaaaatgaggccgatctgatcaaggcgtatccaacgtcaaaagccgaagcggaagcggcgttcggcaatagcgccttgctcatggaaaagtatctcccgcaggtgcggcacgtcgaaatccaggtgctcgcagaccagtatggtcatgccattcaccttggtgaacgcgattgttcgtcgcaacgccgccatcagaagatcgtcgaagaggcgccgtcgccagccgtcaatcccgatctgcgcgcgcgcatgggtgaagcggcgctcaaaggggtgcgcgccatcaattatgtcaatgcggggacgatggagtttctgctcgatcccgacggcaacttctacttcatcgagatgaacacccgcatccaggtcgagcatccggtcaccgaaatggtcaccggcattgatctggtcaaatggcagttgcgcatcgcggctggcgaaccgttgacaatccggcagtcggacgtggtgatgcgtgggcacgcgattgaagcgcggatcaacgccgaagacccggaccgcgatttcatgccgtcgggtggtgagatcgagtactatcttccgcctggcggaccgggggtgcgcgtcgattcgcatctctacgccggatattcgccgccgggatattacgattcactcctggcgaaattgatcgtctggggcgcggatcgcaatgaggcgcttgctcgcctcgaacgcgctctgcgcgaattcgtgatcactggaatctacaccaccatcccgttcaccctggcaatgctcgaagatccgccgttccgcgaagggcgaatctcgacgcgctacgtccccgatctggttcagcgtatcaaggagagtaaactcgagtag

SEQ ID NO: 91 - DNA, 1368 bp, Herpetosiphon aurantiacus
atgttacgcaaaattttaattgccaatcgtggtgaaattgcggtgcgaattattcgtgcttgccacgagctaggcatcaaagcagttgccgcctattccgaggccgatcgcgattcgctggcggtgcgtatggccgatgaggcgatttgtattggcccgccaccacctgccaaatcctatttgaatgcgccagccttgattagcgctgcgctgattagcgattgcgatgggattcacccaggttatggctttttgtcggaaaacccctattttgctgaaagctgccgtgagtgtggtctgacttttattggcccttcagccgattcgattcagcgcatgggcgataaagcgctggccaagcaagccatgaagttggctggcctgccgcttgtgcctggcaccgaaaaccccttgaccagcgttgaagaagctcaaagccttgctgatggtattggctacccggttttgctcaaagctgtggctggcggtggcgggcggggcatgcgcgtggtcaatcagcctgatgaattggcccgagcttttaatactgcccgcgctgaggccgaagctgcctttggccgtggcgatttgtatatggaaaaatacttgccagtggtgcgccacgttgaaattcagattttggctgatcaacatggccatgcaattcaccttggcgagcgtgattgctcgttgcaacgtcgccaccaaaaagtggtggaagaaggcccatcgcctgccttgaccccagaattacgccagaaaatgggcgaagccgccttgcatggcgtgcgcgaaattggctactacaacgctggcacaatggaatttttactcgatcatcagggaaatttctattttatggaaatgaacacccgtttgcaggttgagcaccctgtgactgaatggctgaccggacttgatctggttaagtggcaaattcggattgcttccggcgaacgcttgacgctcactcaggatgacattaaaatacgcgggcatgcgattgaatgtcggattaatgccgaagatgccgaccgtgattttatgcctgctggcgggactgtcgatctctacttgccgccaggtggcccaggggtacgggtcgattcgcatctttattcaggttatcgcactcctaccaactacgattcgatgcttgccaaagtgatcgtctggggggaaacgcggcttgaggcaattgaacgtatgcggcgagcattaagcgaatgtgtgatcaatggcattacgaccaccttgccatttcaactgcgcatgatgaacgagccagcttttgtgagcggcgatgttgcaacgcacaccttggctgatattttaaatcaacaggctgccaaagaagcgacagcgtag

SEQ ID NO: 92 - DNA, 1488 bp, Nitrosopumilus maritimus
atgattgagaaagtacttattgcaaacagaggagaaattgctctcagagtaattagaacatgtaatgcattaggtatcaagactgttgcagtatactctgatgaggattacaattctttacatgtaaagaaagctgatgaatcttatcacattggggaagcagctcctgcaaaatcttatcttaatcaagaaaaaattcttgaagtgatgctgtcatctggtgcagatgctgtacatccaggttatggtttcctttctgaaaatgatgactttgcaagattatgtgaaaaaaataaaattaatttcattggtccatctgctgactccatgaatctatgtggtgataagatggaatgtaaagcagcaatgttaaaagctcaagttccaacagtacctggaagtccaggattagtagataccgcagaagaagcagaaaaaattgcaaatgaaattggttatcctgtactcttgaaatcagtttatggtggtggaggtcgtggaatcagattagttaccactgatcaagaactacgagaaggttttgaaacagtaacttctgaatctattgctgctgtaggaaaatctgcaatcattgttgaaaaattcctagaaaaaacaagacacattgaatatcaaatgtgtagagatcatcatggtaatgctgtacacctctttgagagagaatgttctattcaaagaagaaatcaaaaactaattgaacaaactccttcccctgttgtagatgaagcaaaacgagaagagattggtgaattagttgttaaagcagcagaagctgtcaattatactaaccttggtactgcagaattccttagagcagataatggtgagttttactttattgagattaatgcaagactccaagtagaacatccaatcagtgaaatggtttcaggattagactttgtaaaattacaaattgatattgcaaacggtgaaacacttccattcaaacaaaaagatctaaagatgaatggttatgcaattgaatgtagaatcaatgctgaagacacatttttggactttgcaccttcaactggacctgttccagatgtaacgattcctgcaggaccaaatgtcagatgtgacacttatctatatcctggatgtacagtatctccattttacgattcattgatggcaaaactctgtacatggggaccaacatttgaagaatctagaactagaatgcttactgcattaaatgatatgtatgttcaaggtgttgaaacatcaattccactttacaaaacaattctaaactctgaagaatacaaaaacggtgaactatcaactgactttttgaaacgttatggcatgattgataaactatctgaagaccttaagaaagaaaaagaagacaagagtgaagctgctttggctgcagccattattcattctgaatactttaagaacagagttcaaaatgataacgcaagcagtgcaacttggaaaaataaattggattga

SEQ ID NO: 93 - DNA, 1488 bp, Nitrosoarchaeum limnia
atgattgaaaaagttcttatcgcaaatagaggagaaattgctttacgtgtaattaaaacatgcaaagcgcttggaatcaaaactgttgcagtgtactctgatgaagattataattcgttacatgttaaacaagcaactgaagcatatcatattggtgaggctgctcctgcaaaatcttatctgaatcaagaaaaaattcttgaaaccattttatcttctggtgctgatgctattcatcctggctatggttttctttcagagaattctgactttaccggcaaatgtgaaaaaaataaaataaattttattggtccgtcatctgtatctatggaactttgtggcgataagatgcagtgtaaagctgctatgttaaaggcaaaagttccaacagtaccaggtagtcccggtcttgttaaagatgttgaagaagcattaaaaattgcaaatgatatctcataccccgtattattaaaatcagtttttggtggaggtggtcgtggaattagattagtaaataatgataaagaattacgagagggttttgaaactgttacaagtgagtctatctctgctgttggtaaatctgcaatcattgttgaaaaatttcttcaaaaaactaggcatattgaatatcagatggctcgagacaaacatggtaatgcggttcatatctttgaaagagaatgctctatacaaagacgtaatcaaaaactcatagaacaaacaccatcacccgtagttgatcaaaagacacgagatagaataggcgaattggtagtaaaagcatcagaagctgttgattatactaacttgggcactgcagaatttctcagagctgataatggtgagttttatttcattgagattaatgctcgcttgcaagttgaacatccgatttctgaattagtttctggtttagattttgtaaaacttcaattagatattgctaatggtgaaccacttccattcaaacaaaaggatctaaaaatgaatgggtatgcaattgaatgtagaattaacgcagaagatacattcttagattttgctccttctacaggaccagttccagatgtgacaattccttctggacccagtgttagatgtgatacatatctgtatcctggatgtactgtatctcctttttatgactctttaatggctaaattatgtacttggggtcaaacatttgaagaatctagaactagaatgcttaatgcattaaatgatttctatatccaaggtgttgaaacttcaattcctctttacaaaacaattctaaatacagatgaatacaaaagtggaagtctctctaccgatttcttgaatcgttataaaattattgatagattaaaagaagatcttaaaaaagaaaaaatagaaaaaagtgatgctgcattggcagctgcaattatttattctgaatactttaagagcagagtacaaaattctactcctgataactccaattggaaaaataaattaggttga

SEQ ID NO: 94 - DNA, 1491 bp, group I marine Crenarchaea
atgataagtaaggtactaattgctaaccgtggcgaaatcgcattacgtgtaattaagacatgtaaagcacttggaataaagactgtagctgtatattctgacgaagacagaaattctcttcatgtaaaaaacgctacagaatcatatcatataggtgaggctgctcctgctaaaagctaccttaaccaagaaaaaattcttgacgtgattttatcctctggtgctgatgcagttcatcctggttatggatttttatcagaaaactcagaatttgcaggtttgtgcgaaaaaaacaaagttacattcattggaccatctgctgcatcaatggatctttgtggtgataagcagcaatgtaaggctgcaatgcttaaggcgaaagtaccaaccgtgcctggaagcccagatttagtaaaagatgctgatgaggcagaaaaaattgcaaatgaaattggctatcctgtaatgctaaaatctgtttatggtggtggtggtcgtggcattagaattgtaaacactgatcaagaattacaaggtgcatatgaaacagttacaggagagtcaattgcagcagtaggaaaatctgcaattcttgtagaaaaattccttgcacaaactagacatattgaataccaacttgccagagacaagcatggtaatactgttcatatatttgagagagagtgttcaattcaaagacgtaaccagaaattaattgaacaaactccttcaccaatagttgatcaagaaacaagagatagaattggaaaactagttgtaaatgcggcagaggcagttgattatactaatcttggtacggtagaatttctcagagcagacaatggagaattttactttctagagattaacgctagattacaagttgaacatccaatcaccgaatttgtatccggattagaccttgtgaaactacaattagatattgcaaacggtgagccaattccattcaaacaatctgatcttaaaatgaatggttatgcaattgaatgcagaataaatgcagaagatacattcttggactttgctccatcgacaggcccgataccaaatgttactatcccatcaggtcctggcgttagatgtgatacctatctttacccgggttgtaccgtatcagcattttatgattctcttatggcaaagcttgtcacatgggggcagacatttgaagagtcaagactgagaatgcttaatgcattaaatgatttttacatccagggcgttgaaacttcaattcctctttacaaaacaattttagaaacggaagaatacaaaaatggagaactctcaacaaactttttgaaaagatttgatataattgagagactaaaagaagacatcaaaaaacaaagaaaggataaacatcttgctgcaattgctgcagcagtgatgcattcaacatttttccaaagcagggtgcaatcatcaactccgaagaatccacgatggaagagtcgaatggatagatag

SEQ ID NO: 95 - DNA, 1491 bp, group I marine Crenarchaea
atgataagtaaggtactaattgctaaccgtggcgaaatcgcattacgtgtaattaagacatgtaaagtacttggaataaagactgtagctgtatattctgacgaagacagaaattctcttcatgtaaaaaacgctacagaatcatatcatataggtgaggccgctcctgctaaaagctatcttaaccaagaaaaaattcttgacgtgattttatcctctggtgctgatgcagttcatcctggttatggatttttatcagaaaactcagaatttgcaggtttgtgcgaaaaaaacaaagttacattcattggaccatctgctgcatcaatggatctttgtggtgataagcagcaatgtaaggctgcaatgcttaaggcgaaagtaccaatcgtgcctggaagcccagatttagtaaaagatgctgatgaggcagaaaaaattgcaaatgaaattggctatcctgtaatgctaaaatctgtttatggtggtggtggtcgtggcattagaattgtaaacactgatcaagaattacaagatgcatatgaaatagttacaggagaatcaattgcagcagtaggaaaatctgcaattcttgtagaaaaattccttgcacaaactagacatattgaataccaacttgccagagacaagcatggtaatgctgttcatatatttgagagagagtgttcaattcaaagacgtaaccagaaattaattgaacaaactccttcaccaatagttgatcaagaaacaagagatagaattggaaaactagttgtaaatgcggcagaggcagttgattataccaaccttggtaccgcagaatttctcagagcagacaatggagaattttactttctagagattaacgctagattacaagttgaacatccaatcaccgaatttgtatctggattagaccttgtgaaactacaattagatattgcaaacggtgagccaattccattcaaacaatctgatcttaaaatgaatggttatgcaattgaatgcagaattaatgcagaagatacatttttggactttgctccatcaacaggtccagtaccaaatgttactatcccatcaggtcctggcgttagatgtgatacctatctttacccgggttgtaccgtatcagcattttatgattctcttatggcaaagcttgtcacatgggggcagacatttgaagagtcaagactgagaatgcttaatgcattaaacgacttttacattgagggtgtagaaacttcaattcctctttacaaaacaattttagaaacggaagaatacaaaaatggagaactctcaacaaactttttgaaaagatttgatataattgagagactaaaagaagacatcaaaaaacaaagaaaggataaacatcttgctgcaattgctgcagcagtgatgcattcaacatttttccaaagcagggtgcaatcatcaactccgaagaatccacgatggaagagtcgaatggatagatag

SEQ ID NO: 96 - DNA, 1569 bp, Acidianus hospitalis
atgtcccaagaagaaaaaactatggataaattaatacaagaattaaaaaatatgaaagaaaaggcatatcaaggtggaggagaagacaggataaaagcccaacatagcaaaggtaagttaactgccagagaaaggttagctctgctcttcgacgaaggaacttttaacgaaataatgacttttgctactactaaagctacagaatttggattagataaaatgaaaatgtacggcgacggtgtagtaacaggttggggtaaagtagacggcaggaccgtttttgcatattctcaagactttactgagttgggcggaactttaggggaaatgcatgcaaataaaatagccaaagtttacgagttagcactaaaggttggagcaccagttgtaggaataaatgactctggaggagcaagaattcaggaaggtgcagtagcgcttgaaggctatggtcaagtattcaagatgaacgtcatggcttctggagtaattcctcaaattactataatggcagggcccgcagcaggaggagcagtatattctccagctttgactgatttcattattatgattaaaggtgacgcttactacatgttcgtcaccggtccagaaataactaaagtagtattaggagaagaggttagcttccaagacctaggaggagcagttattcatgctaccaagtctggagtagttcatttcctggcggagaatgaacaggatgcaattaacattgccaagaggttactttcctatttaccttcaaataacatggaagaaccaccgtttatggacactggagatcctgccgatagagatgttaaagacgttgagcaaatagttcctactgattctgcaaagccgtttgatatgaaggaaatcatatataggatagtagataacggagaattcctagaagttcataaacattgggcacagaatattgtagtaggatttgcaagaattgcaggaaacgttgtaggaatagttgcaaataactctcagtacttaggagcggctatagacattgacgctgcagacaaagctgcaagattcataaggttctgtgatgcatttaacattcctttaataagccttgtggatactccaggatatattccgggcactgaccaagaatataagggaataattaggcacggcgcaaaaatgctttatgcgtttgcagaggcgacagtacctaagataacagttataataaggaaatcctacggaggtgcacatattgcaatgagtataaagagtttaggagcagatctagtttatgcttggcctacagctgaaattgctgtaactggtccagaaggtgctgtaaggatattatacaagaaggaaatacaagcatcaagcaatccggacgaattcataaagcagaagatcgcggaatataggaaattatttgccaatccttactggtcggcagagaagggattgatagatgacgttatagaacctaaggatactaggagagtaatagtttcagcattagagatgctaagaaataagagggaatatagatatccaaagaagcatggtaatataccgctctaa

SEQ ID NO: 97 - DNA, 1575 bp, Metallosphaera sedula
atgactgcaacttttgaaaaaccggatatgtcaaaactagttgaggaattgagagccctaaaggccaaggcttacatgggtggaggagaggagagagtacaggctcaacatgctaagggcaagctgacagcgagggagaggttaaatctcctattcgatgaggggacctttaacgaggtcatgacctttgccacgacaaaggctactgagtttggattggataaaagcaaggtctacggagacggcgtagtaactggatggggacaggtcgagggaaggactgtatttgcatttgcccaggacttcacgtctataggaggcacgctaggggagactcacgcgtctaaaatagctaaggtttatgagctcgcattaaaggttggagcacctgtagttgggataaatgattctggaggagccagaatacaagagggcgcagtcgcactagagggttacggtacggtctttaaggccaacgtgatggcatctggcgtagttccccagataaccataatggctggtccagctgcgggaggtgcagtttactcaccagccctcactgacttcataatcatgataaagggcgacgcctactacatgtttgtgaccggtccagagatcacaaaggtggtgttgggtgaggacgtttcattccaagacctaggtggagccgtaattcacgcaactaaatcaggcgtggttcacttcattgcggagaacgaacaagattccattaacataaccaagaggttgctctcttacctaccctctaacaacatggaggagccacccttcatggacacgggagaccctgcggacagggaaatgaaggacgtggagagcgttgttccaactgacaccgtcaagccctttgatatgagagaggtaatatacaggactgtggacaacggcgagttcatggaggtgcagaagcattgggctcagaacatggtggttggatttggaagggtagccgggaacgtggtaggtatagtagcaaataactccgcccatctgggggcagccatagatatagacgcctcagacaaggcggccaggttcataaggttctgtgacgcttttaatattcccttgattagcttggtggacactcctggttacatgccgggaacagaccaggaatataagggcatcattaggcacggagcaaagatgttgtacgcctttgctgaggcaacagttcccaaggtcactgtggtggttagaaggtcctacggtggcgctcacatcgccatgagcataaagagccttggagcggatctcatatatgcttggccctctgcagagatagcggtgactgggcctgagggggccgtgaggatcctgtacaggagagaaattcagaacagcaagtctcctgacgatctcatcaaggagagaatagctgagtacaagaagttgttcgccaacccctattgggcagctgagaagggattgattgacgacgtaatagagcccaaggatacgaggaaggtaatagcgtcagccttgaagatgttaaagaacaagagggagttcaggtaccccaagaagcatggaaatatacccctctaa

SEQ ID NO: 98 - DNA, 1575 bp, Acidianus brierleyi
atggctataacttctgaaaaatcagatatggataaaataatagctcagttaagggaaataaaagctaaagcttttcaagggggtggagaagacaaaattaaagcacagcacgataaaggtaaattaactgctcgagaaagattagccttattgtttgacgaaggttcttttaatgaaattatgccattagcaactacaagggctacagaattcggacttgataaaaacaagttttatggtgatggagtagttactggttggggaaaaatagaaggaaggacagtatttgctttttctcaggattttacagaattaggaggtactctaggtgaaacgcacgcaaacaaaattggtaaagtttatgaactagctctaaaggtgggagctccggtaataggcattaatgattctggtggtgcaagaattcaagaaggtgctatagcattagaaggatatgctacagtattcaagatgaacacattggcttcaggagttattccacagattactataatggccggcccagcagctggtggtgctgtatattctccagcacttactgattttattataatgattaaaggagatgcttattacatgtttgtaactggtccagaaataactaaagtagttttaggagaagaagtaacatttcaagatttaggaggagctgtagttcatgctactaaatctggggttgttcatttcttagccgaaaatgaacaagacgctataagtatagctaagcgattactgtcttatcttccttctaataacatggaagatccgccctatatggatactggagatccttcagatagggaaactaaagacgttgaaagtattgtacccacagattcagctaaaccttttgatatgagagaggtaatatacaggattgtagataatggagaatttatggaggttcatagacattgggctcagaatattgtagtaggatttgctagagttgctggaaatgtaataggtatagttgctaataattccaatacactaggtgcagctatagatatagatgcggcagataaagctgctagatttatcagattttgtgatgcgtttaatattcctctacttagtttagttgatactcctggctatatccctggaacagaacaagaatataagggaataataaggcatggggctaaaatgttatatgcattttctgaagctactgtaccaaaaatttcagttattataagaaagtcttatggaggtgcacatatagcaatgagcataaagaatttaggtgcagatttggtttacgcttggcctactgcagaaatagctgttactggtcctgaaggagcagtaagaatattatataagagagatattcaaaattctcaaaatcctgatgagttcctgaaacaaaaaatagccgaatataagaaactgttcgccaacccatactgggctgcagaaaaaggtcttatagatgatgtaatagagcctaaagataccagaaggataattgttaacgctttatccatgcttaaaaataagagagaatacagatatcctaaaaaacatggaaatattccattataa

SEQ ID NO: 99 - DNA, 1575 bp, Metallosphaera cuprina
atgactgcaacttttgaaaagcaggatatgtcaaaactgattgaggagttgaggaacctcaaagcaaaggcgtataagggaggaggggaagagaggattcaattccagcataacaagggtaagctgaccgcaagggagaggcttaacctcctctttgatgagggaacgttcaacgaagttttgacattcgccacaacaaaagctactgagtttaacttggataagaacagagtttacggcgacggcgtagtaaccgggtggggtcaagttgagggaaggaccgtcttcgcctttgctcaagatttcacctccattgggggaacgttgggcgagactcacgcctcgaagatagctaaggtttacgagctagccctaaagacaggagccccggtaattggtataaacgactcaggaggagctaggattcaagagggagccgttgcgctagaagggtacggcacggtctttaaagccaacgttatggcgtcaggggttatcccgcagataaccattatggccggaccggcagcaggaggtgctgtctactcgccagctttaaccgatttcattatcatgattaagggagaagcttattacatgttcgttacggggccagagatcaccaaggtggttctaggtgaggaagtctctttccaagatttaggcggggcagtgattcacgctactaagtccggagtagtccacttcgtagccgagaacgaacaggattcgattaatattgcgaagaggctcctctcgtacttaccatcaaataacatggaggaaccgcccttcgtagacacaggagatcctatggatagggagatgaaagatgtggagactatcgtacccacagacacagttaagcccttcgatattagagaagtgatctataggactgtggataacggcgagttcatggaagttcagaagcattgggctcagaacatggtagttggtttcgccaggatggccggtaacgtcgtgggtatcgtagctaacaactccgctcatttgggggctgccatagatatagacgcttcagataaggctgctaggttcataaggttctgcgatgcgtttaacattccactcgtaagcctcgtcgacacacctggatatatgccaggtactgatcaggagtataagggtataatcaggcatggagcgaagatgctgtacgccttctctgaagcaaccgtgcccaaagtcactgtagtggtcagaagatcttacgggggagctcacatagctatgagtataaagagtctaggggcagacttaatttacgcctggccatcagctgagatagcagttacaggtcctgaaggggcggtgaggatattatataggagggaaatacaaaacagtaaatcgcctgacgatttcataaaggagaggatagccgagtataagaagctattcgctaacccgtattgggcggccgagaaagggttaatagatgacgtgatagaacctaaagatactaggaaggtgatagtctccgcgttgaggatgttaaagaacaagagagagttcagatatcctaagaaacatggaaatattccactttaa

SEQ ID NO: 100 - DNA, 1572 bp, Sulfolobus solfataricus
atgtctctatatgaaaaacctccaatggataaattaatagaagatctgaaaatattgaaggaaaaagcatataaaggcggaggagaggagagagtaaactttcagcatagcaaagggaaactaacagcgagggagaggttaaatctattattcgacgaaggaacattcaatgaaatattaacatttgccactactagagcaacagaattcgggttagataggaataagttttacggagatggagtgatagctggatggggaaaagtagatggcagacaagttttcgcttacgctcaagactttacggttctcggaggaagtctaggagaaacacatgcaaacaagatagttagagcttatgagctagccctaaaggttggcgctccggtaataggaataaacgattccggaggtgccagaatacaagaaggcgcattatctctagaaggatatggtgcagtgtttaagatgaacgtaatggcgtctggagtaattccccaaattaccattatggcaggaccggcagctggaggggctgtctactcgcctgctctaaccgacttcttaataatgataaaaggagacgcgtattacatgtttgtaaccggcccagagattactaaagtgtcaataggggaagaagttagttaccaagatctaggtggtgcaatagttcacgcaaccaagtctggagtagttcattttgtagctgaaaacgaacaagatgcgataaatatagctaagaggttactctcctatttgccttcaaataatatggaagagcccccatatattgatactggtgatcccgctgatagagaagtacaaggtgcagagtcaatagtgcctactgactcagtaaaaccattcgacataagagacctaatatataatatagttgacaatagcgaattcttggaagttcacaaattatgggcacaaaatattactgtagggtttggaaggataaatggaaacgttgtgggcattattgctaataattcagcatactatggaggagcaatagatattgatgcagcggataaagctgccagatttatcagattctgtgatgcgttcaacataccgttaataagtcttgtagatacgcctggttatgtacccggaacagatcaagaatataaagggataataagacatggtgctaaaatgttatacgcgtttgctgaggctacagtaccaaagataacagttattgtaagaaggtcttatggtggtgctcacatcgcaatgagtattaaaagcttaggtgctgacctagtttatgcttggccatctgcagaaatagccgtaactggcccagaaggtgcagttagaatattatataggagggaaatacaaaatgcgcaaaatccggaagaattcttaaaacaaaaaatagccgagtacaagaaattattcgcgaatccttactgggcagccgagaagggtcttatagacgatgttattgagcctaaagacactagaaaagtaatatctagaggattagaaatactaagaaataaaagagaattcagatatcctaagaaacatggaaatatacctctatag

SEQ ID NO: 101 - DNA, 1572 bp, Sulfolobus tokodaii
atgtctatgtatgaaaaacctccagttgaaaaattaattgaagaattaagacaattaaaagaaaaagcgtataagggaggaggagatgaaagaatacaattccagcacagtaaaggtaagcttacagctagagagagattagcacttttgtttgatgatggtaaatttaatgaaattatgacttttgcaactaccagagctacggaattcggtctagataagcagagattctatggagatggtgttgttactggttggggtaaagttgacggtagaacagtttttgcgtatgcccaagattttacagtattaggaggaagcttaggagagacacatgcaaataagattgttagagcttatgaattagctctaaaagtaggagcaccagttgtgggtattaatgattctggtggtgcaagaatacaagaaggtgcattatcgttagaaggatatggtgctgtattcaaaatgaatgtaatggcttctggtgtgattccacaaataactattatggctggaccagcagcaggtggtgcggtttattcgccagcactaacagatttcataattatgattaaaggagacgcgtactatatgtttgtaacggggccagaaattactaaagtagttttaggagaagaagtatctttccaggatttgggtggagctgtcgttcatgcaacaaagtccggtgtagtacatttcatggttgatagtgaacaagaagcaattaatttaactaaaagactattatcatatttaccatcaaataacatggaagaacctccttatattgatactggagatccagcagatagagatgcaactggagtagaacaaatagttcctaatgatgcggcaaaaccctataacatgagggaaataatttacaagattgtagataatggtgaattccttgaggttcataaacattgggcacaaaacataatagtaggattcgcgagaattgcgggtaatgttgttggaatagtagctaataacccagaagaatttggcggttctatagatatcgatgctgctgataaggcagcaagatttattagattctgtgatgcgttcaacatacccctaattagcttagtagatactccaggttacgtgccggggactgatcaagagtataaaggcataataaggcatggtgcaaaaatgctatatgcatttgctgaagcgactgtacctaaaattacagtaattgttagaaaatcttatggaggagcacatatagcaatgagcataaagagtttaggagcagatttagtatatgcttggccaacagccgaaattgctgtaacgggaccggaaggtgcagtaagaattctttatagaaaagaaattcaacaagcttcaaatccagatgatgtattaaagcaaagaatagcagaatataggaagttgtttgcaaatccgtactgggctgctgagaagggactggttgatgatgtcattgagccaaaagatactagaagagtaattgtagctggattagaaatgctaaagactaagagagaatataggtatcctaagaaacatggtaatataccattatga

SEQ ID NO: 102 - DNA, 1572 bp, Sulfolobus islandicus
atggctctatatgaaaaaccttcaatggataaattaattgaagatttgaaaatattaaaggaaaaggtatataagggtggaggagaagagaagataaacttccagcatggtaaaggtaaactaacggcaagggagagattaaatcagctgttcgatgaaggaaagttcaatgaaatattgacttttgccactactagagcaactgaatttgggttggataagaataggttttacggagatggagtaatagctggatggggaaaaattgatggtagacaagttttcgcctatgctcaagactttacgatactcggaggaagcctaggagaaacacatgcaaataagatagttagagcttatgagctagccctaaaagttggtgctccagtaataggaataaacgattctggtggagcaagaatacaagagggtgcactatcgctagaaggatatggtgcagtgttcaagatgaacgtaatggcctctggagtaattccacaaattaccattatggcaggaccggcagctggaggtgcagtctactctcctgctcttactgacttcttaataatgataaaaggagacgcatattacatgtttgtaaccggaccagagattactaaagtgtcaataggcgaagaagtgagttatcaagacctaggtggtgcaataatacacgcaacaaaatctggagtagttcactttgtggctgaaaatgaacaagatgcaataaacataactaagaggttactgtcttacttgccttcaaacaatatggaagaacctccatatgttgataccggtgatcctgctgatagagcagtgcagagtgctgaatcgattgtacctacagactcagtaaaaccatttgatataagagacttaatatacaatatagttgacaatagcgaattcctggaagttcacaaattgtgggcacaaaatattactgtgggatttggaaggataaatgggaacgtcgtgggaattgtcgcaaataattcagcatattatggaggagcaatagatattgacgctgcggataaggctgctagatttatcagattctgcgatgcgttcaacataccggtaataagcttagttgacactccaggatatgttccagggacggaccaagagtataaagggataataaggcatggtgctaaaatgttatacgcattcgctgaggccacagtaccaaaaattacggttattgtaagaaggtcttatggtggtgctcatattgcaatgagcattaaaagtttaggtgctgatttagtttatgcttggccttctgcggagatagctgtaactggtccagaaggtgcagttagaatattatatagaagggaaatacaaagtgctcaaaatccagaggaactcttgaaacaaaaaattacagaatacaagaaattgttcgctaatccttattgggcagctgagaaaggtttaatagatgacgtcattgagcctaaggacaccagaaaggtaatagctagaggattagaaatgttaagaaataagagggaattcagataccccaagaaacatggcaacatacctctctga

SEQ ID NO: 103 - DNA, 1572 bp, Sulfolobus islandicus
atggctctatatgaaaagccttcaatggataaattaattgaagatttgaaaatattaaaggaaaaggtatataagggtggaggagaagagaagataaacttccagcatggtaaaggtaaactaacggcaagggagagattaaatcaactgttcgatgaaggaaagttcaatgaaatattgacttttgccactactagagcaactgaatttgggttggataagaataggttttacggagatggagtaatagctggatggggaaaaattgatggtagacaagttttcgcctatgctcaagactttacgatactcggaggaagcctaggagaaacacacgcaaataagatagttagagcttatgagctagccctaaaagttggtgctccagtaataggaataaacgattctggtggagcaagaatacaagagggtgcactatcgctagaaggatatggtgcagtgttcaagatgaacgtaatggcttctggagtaattccacaaattaccattatggcaggaccggcagctggaggtgcagtctactctcctgctcttactgacttcttaataatgataaaaggagacgcatattacatgtttgtaaccggaccagagattactaaagtgtcaataggcgaagaagtgagttatcaaaacctaggtggtgcaataatacacgcaacaaaatctggagtagttcactttgtggctgaaaatgaacaagatgcaataaacataactaagaggttattgtcttacttgccttcaaacaatatggaagaacctccatatgttgataccggtgatcctgctgatagggcagtgcagagtgctgaatcgattgtacctacagactcagtaaaaccatttgatataagggacttaatatacaatatagttgacaatagcgaattcctggaagttcacaaattgtgggcacaaaatattactgtgggatttggaaggctaaatgggaacgttgtgggaattgtcgcaaataattcagcatattatggaggagcaatagatattgacgctgcggataaggctgctagatttatcagattctgcgatgcgttcaatataccggtaataagcttagttgacactccaggatatgttccagggacggaccaagagtataaagggataataagacatggtgctaaaatgttatacgcattcgctgaggccacagtaccaaaaattacggttattgtaagaaggtcttatggtggtgctcatattgcaatgagcattaaaagtttaggtgctgatttagtttatgcttggccttctgcggagatagctgtaactggtccagaaggtgcagttagaatattatatagaagggaaatacaaagtgctcaaaatccagaggaactcttgaaacaaaaaattacagaatacaagaaattgttcgctaatccttattgggcagctgagaaaggtttaatagatgacgtcattgagcctaaggacaccagaaaggtaatagctagaggattagaaatgttaagaaataagagggaattcagataccccaagaaacatggcaacatacctctctga

SEQ ID NO: 104 - DNA, 1572 bp, Sulfolobus islandicus
atggctctatatgaaaaaccttcaatggataaattaattgaagatttgaaaatattaaaggaaaaggtatataagggtggaggagaagagaagataaacttccagcatggtaaaggtaaactaacggcaagggagagattaaatcagctgttcgatgaaggaaagttcaatgaaatattgacttttgccactactagagcaactgaatttgggttggataagaataggttttacggagatggagtaatagctggatggggaaaaattgatggtagacaagttttcgcctatgctcaagactttacgatactcggaggaagcctaggagaaacacacgcaaataagatagttagagcttatgagctagccctaaaagttggtgctccagtaataggaataaacgattctggtggagcaagaatacaagagggtgcactatcgctagaaggatatggtgcagtgtttaagatgaacgtaatggcttctggagtaattccacaaattaccattatggcaggaccggcagctggaggtgcagtctactctcctgctcttactgacttcttaataatgataaaaggagacgcatattacatgtttgtaaccggaccagagattactaaagtgtcaataggcgaagaagtgagttatcaagacctaggtggtgcaataatacacgcaacaaaatctggagtagttcactttgtggctgtaaatgaacaagatgcaataaacataactaagaggttattgtcttacttgccttcaaacaatatggaagaacctccatatgttgataccggtgatcctgctgatagggcagtgcagagtgctgaatcgattgtacctacagactcagtaaaaccatttgatataagggacttaatatacaatatagttgacaatagcgaattcctggaagttcacaaattgtgggcacaaaatattactgtgggatttggaaggataaatgggaacgttgtgggaattgtcgcaaataattcagcatattatggaggagcaatagatattgacgctgcggataaggctgctagatttatcagattctgcgatgcgttcaatataccggtaataagcttagttgacactccaggatatgttccagggacggaccaagagtataaagggataataaggcatggtgctaaaatgttatacgcattcgctgaggccacagtaccaaaaattacggttattgtaagaaggtcttatggtggtgctcatattgcaatgagcattaaaagtttaggtgctgatttagtttatgcttggccttctgcggagatagctgtaactggtccagaaggtgcagttagaatattatatagaagggaaatacaaagtgctcaaaatccagaggaactcttgaaacaaaaaattacagaatacaagaaattgttcgctaatccttattgggcagctgagaaaggtttaatagatgacgtcattgagcctaaggacaccagaaaggtaatagctagaggattagaaatgttaagaaataagagggaattcagataccccaagaaacatggcaacatacctctctga

SEQ ID NO: 105 - DNA, 1572 bp, Sulfolobus solfataricus
atggctctatatgaaaagccttcaatggataaattaattgaagatttgaaaatattaaaggaaaaggtatataagggtggaggagaagagaagataaacttccagcatggtaaaggtaaactaacggcaagggagagattaaatcagctgttcgatgaaggaaagttcaatgaaatattgacttttgccactactagagcaactgaatttgggttggataagaataggttttacggagatggagtaatagctggatggggaaaaattgatggtagacaagttttcgcctatgctcaagactttacgatactcggaggaagcctaggagaaacacacgcaaataagatagttagagcttatgagctagccctaaaagttggtgctccagtaataggaataaacgattctggtggagcaagaatacaagagggtgcactatcgctagaaggatatggtgcagtgttcaagatgaacgtaatggcttctggagtaattccacaaattaccattatggcaggaccggcagctggaggtgcagtctactctcctgctcttactgacttcttaataatgataaaaggagacgcatattacatgtttgtaaccggaccagagattactaaagtgtcaataggcgaagaagtgagttatcaagacctaggtggtgcaataatacacgcaacaaaatctggagtagttcactttgtggctgaaaatgaacaagatgcaataaacataactaagaggttattgtcttacttgccttcaaacaatatggaagaacctccatatgttgataccggtgatcctgctgatagggcagtgcagagtgctgaatcgattgtacctacagactcagtaaaaccatttgatataagggacttaatatacaatatagttgacaatagcgaattcctggaagttcacaaattgtgggcgcaaaatattactgtgggatttggaaggataaatgggaacgttgtgggaattgtcgcaaataattcagcatattatggaggagcaatagatattgacgctgcggataaggctgctagatttatcagattctgcgatgcgttcaatataccggtaataagcttagttgacactccaggatatgttccagggacggaccaagagtataaagggataataaggcatggtgctaaaatgttatacgcattcgctgaggccacagtaccaaaaattacggttattgtaagaaggtcttatggtggtgctcatattgcaatgagcattaaaagtttaggtgctgatttagtttatgcttggccttctgcggagatagctgtaactggtccagaaggtgcagttagaatattatatagaagggaaatacaaagtgctcaaaatccagaggaactcttgaaacaaaaaattacagaatacaagaaattgttcgctaatccttattgggcagctgagaaaggtttaatagatgacgtcattgagcctaaggacaccagaaatgtaatagctagaggattagaaatgttaagaaataagagggaattcagataccccaagaaacatggcaacatacctctctga

SEQ ID NO: 106 - DNA, 1536 bp, Sulfolobus acidocaldarius
ttggaagaattgaaaaagaagaaggaattggtatacaagggaggaggagaagagagaattaaagcacagcatgataaggggaaactgactgcaagagagagactatcattattgtttgatggtaatacatttcaggaatttatggggtttgcaacaacaaaggctacggaattcggtctagataagaacaaggtctatggggacggtgtggtcacgggatggggaaaagtagagggaagaactgttttcgcctacgcacaagacttcatatcattaggaggtacattgggcgaggtacatgctaacaagattgccagagtttatgagttagcccttaagacaggtgcacctgttgtaggaataaatgactcaggtggagctagaatacaggaaggagctgttgcactggaaggctatggtgcagtgttcaagatgaatgttatggcatcaggtgttataccacagataactattatggctggacctgctgcaggaggtgcagtttattctccagcattaacagatttcattataatgattaaaggagatgcttactatatgtttgtgacaggaccagaaataactaaggtagctctaggtgaagaagtcagttatcaagatctaggaggagcaatagtgcattcaactaaatctggtgtgatacacttcatggctgaaaatgaacaagacgctataaacataactaagaaattgctgtcatacctaccctcaaacaacatggaagaaccaccatacatagatacaggagacgctgcagacagagacgtatcaggagctaatgatataattccaacagaccccgtaaagccatatagtatgagggagttaatatataggactgtcgataacggcgagttcatggaagttcataagtattgggcaaacaatatgatcattggattcgcaagaataggaggtaatgttgttggtatagtagccaataatccggaggagttcggtggtgcaattgatgtagatgcagctgataaagcagctaggtttataaggttctgtgacgcgttcaatatacccttaataagcctagttgacactcctggttacgttccgggcactgagcaggagtataaggggattataagacatggggctaagatgttgtatgccttcgctgaagctaccgtaccaaagattacggtaatactgagaaaatcttacggcggagctcacatagctatgagcataaagagtctaggcgctgacttagtttatgcttggcctaatgcagaaatagcggttacaggacctgaaggggcagttagaatattgtacaaaagagatttgcagaagatgagtaatcctgaggattatataaaacaaaagatagaggaatacaggagattatttgcaaacccatattgggcagcagaaaagggtctaattgatgatgttatagagcctaaagatacaaggagaataatttactctgctttggaaatgttaaagaacaagagagagtataggtatcctaagaagcatgggaacattccactgtaa

SEQ ID NO: 107 - DNA, 1527 bp, Aciduliprofundum boonei
atgcttgaagaaaaaagaaaaaaagcgatggaaggcggtggagaagaaagaataaaaaagcagcatgagaagggaaaactcacagctagagagagaatcgagaaacttttagacgaaggaacatttgtagaactgggtatgtttgcagagtctcgtgctacagaattcggtatggacaaaaagagatttcctggagatggtgttgtaacaggatatggtacgatagatggtcgactggttttcgtttatgcccaagattttacaattcttggcggctctcttggtgagatgcatgcagagaaaataacaagagttttaaatcttgcattgaaaaacggtgctcctgttatagggcttaacgattctggcggagctagaattcaggaaggtgtggattctctaaagggctatggagagatctttttccgcaacactattgcaagtggtgttgtacctcaaattgcagtgataatgggccccagtgcagggggtgcggtgtactcaccagccattatggactttgtggttatggttgacaaaacatcttatatgttcataactggacctcaggttatcaaggccgttacaggtgaggatgtggattttgagagtttgggtggagcgagagtacacaatgaaaaaagcggaaatgcccacatattcgcaaaaaacgaagaggaagctttgcatcttgtcagagccttactaagctacttaccctcaaataatatggaagaccctccatttatagacacaggagatcctccagacagaatagattacgaattagacaaaatcatacccaaggatcctaaaaaatcctacgatgtgaaagatataatcaatatccttgtggatagaggaacattttttgaaattcatccactctatgcacagaatatagtagtgggatttgcaaggatgggaggtaaggttgttggcatcgtcgcaaatcagccaaagttctatgcgggcgtgcttgatataaatgccagtgataaagctgcgagatttgttagattctgcgatgctttcaatatacctattgttactctcgtggatgtccccggctacatgccaggggtggctcaagagcatggaggaataataaggcatggtgcaaaattactatatgcgtatagcgaggcaacggtaccgaaaataacagtgattctccgcaaagcctatggtggagcatacatagccatgggctccaaacatctgcgcgcagatgttgtttacgcttggcccaatacagaaattgcagtaatgggaccagagggagcagtaaatatcgtatttaggagagagattaaggaggcagagaacccagagaagaggagacaggaattaataaatgaatatcgtgacaaatttgcgaatccctatgtagctgcatcccgtttatatgtggatgacatcatttatccccatgaaaccagacccaagataatccaagctttaaatatgttggagaacaagaaagaggaaaggcctgagaagaagcatggaaatataccactatag

SEQ ID NO: 108 - DNA, 849 bp, chloroflexus aggregans
atggaagagactccgcagcaggaaactatcacaccgtgggatcgcgtgcagttggcgcgccacccacaacgtccacatacgctcgattatatcgctgccctctgtgaggattttgtcgagcttcacggtgaccgtcgctttggtgatgacccggcaatcgtgggtggtatggcgacatttgccggccaaacggtgatggtcatcggccatcagaaggggaacgatacccgtgagaatatgcggcgcaattttggtatgccgcatcctgaagggtaccgcaaggcgcagcggctgatgcgtcacgccgagaagtttggtatgccggtgatatgctttatcgacacacccgccgccgatcctaccaagggatcggaagaacgtggtcaagcaaatgctatcgccgaaagcattatgctgatgacaacgctacgggtaccgagtattgcggtcgttatcggcgagggcggtagcggtggagcattagccattagcgttgctgaccgtattctgatgcaggagaattcgatctattccgtggcgccaccggaagcagcggcttctatcttgtggcgtgacgctgcgaaagcaccagaagcggccaaggcgctaaaattgacggcagccgatctctatgagcttcgtattatcgatgaggtgatcccagaaccgcctggtggtgcccataccgaccgcctaacggcaattaccaccgtcggtgagcggctacgtgcccatctcaccgatttgcaacagcgtgacctcgatacccttctgcgtgaacgctaccagaagtatcgttcgatagggcaattccaagaacagcagatggatttcttcgcccggcgatag

SEQ ID NO: 109 - DNA, 837 bp, Oscillochloris trichoides
atggacgagacgaccaaacacgccgagacccagccccacgaactgacgccctgggatcgcgtgcaacttgcccgccacccccagcgcccgcacacgctcgactatatcggtgccctgtgtgaagattttaccgaattgcacggtgatcgccgctttggcgatgacgcggcgattgtgggtggcttggccacctttgccgcccgtaccgtgatggtgatcggccaccagaagggcagcgacacccgcgagaatgtcaaacgcaacttcggcatgccccacccagagggctatcgcaaagcccagcgtctcatgcgtcaggccgaaaagtttgccatcccggtgatctgttttgtggacacccccgccgccgacccgaagaaggagtcggaggagcgcggccaagccaatgcgattgccgagagcatcctggtgatgaccaacctgcgcgtaccgatcatctcggccgtgattggcgagggtggcagcggtggggcactcgccctcagcgtcgccgaccgcctgatcatgcaggagaacacgatctattcggtggcctcacccgaagcgaccgcctcgatcctctggcgcgactcggccaaggcacccgatgcagcacgggcactcaagctcaccgcccaggatctgtataatctccgcatcattgacgaggtggtcgccgagccagagggcggggcgcaccaggaaccccaagaggcgatccgcatactaggcgagcgcattcttgcccacctcgatgccatctcccagatcgagatcaacaccctgctcaaacagcgctaccagaaataccgcaccatcggtacctacctcgaatag

SEQ ID NO: 110 - DNA, 837 bp, Roseiflexus sp
atgacacaaacacttactccctgggacaaagtgcaacttgcccgccatatgcagcgcccgcgcaccctcgattatattcgcgggttgtgcgacgatttcgtcgaactgcacggcgaccggcgtttcggtgacgatgcagcgattgtcggcggcgtggcgacgtttgaagggcgaaccgtcgtgctggtcgggcaccagaaagggcgcgatgcgcgcgaaaacatccggcgcaatttcggcatgccgcaccccgaagggtatcgcaaagcgctgcggctgtttcagcacgccgagaagttcggcttcccggtgatctgtttcattgacacaccaggcgccaaccccaaccgcgaatcggaagagcgcgggcaggcgaacgcaatcgccgagaacatcctgacgatggcaggtttgaagacgccgatcattgcgtgcgtcatcggtgagggcggtagcggcggcgcgctggcgatcggcgtggcagaccgaatcttgatgctggagcacgcgatctactcggtcgcttcaccggaagcagccgcctcgatcatctggcgcgacgcggcgaaagcgcccgatgcagcgcgtgcgatgcggataacggcgcaggacttgctggaactggggattatcgacgagatcgttcctgaaccatccggcggcgcccatgccgatccgaccacgatggtcgcaacgctgggtgacgctattcgtcggcatctgaaccaactgctggcgttcgatatcgagacattgctccaacatcgttatgagcgctatcgggcgattggacggtacgaggaagatgcatccgggatccttcacactgtgaattga

SEQ ID NO: 111 - DNA, 831 bp, Roseiflexus castenholzii
atgacacagacgcttactccctgggataaagtgcaacttgcacgccatatgcagcgcccgcgcacgcttgattatattcgcgggttgtgcgacgattttgtggaactgcatggcgaccgacgctatggcgatgatgccgcgattgttggcggcgtggggacttttgaaggacggaccgttgtgctggtagggcaccagaaggggcgtgatgcgcgggaaaatattcgacgcaactttgggatgccgcatccagaagggtaccgcaaagcgctacggttgtttcagcatgccgagaagttcggcttcccggtgatctgtttcatcgatacgccaggcgccaatccgaaccgcgagtcggaagagcgcgggcaggcgaatgcgattgccgagaatatcctggtgatggcagggttgaagaccccgatcattgcgtgcgtgattggcgaaggcggcagtggcggcgcattggcgatcggcgttggcgaccgcattctgatgctggagcatgcgatttactcggtcgcctcgccagaagcggcggcatcgatcatctggcgtgatgccgcgaaagcgcccgatgcggcgcgcgcgatgcgtattacagcgcaggatctgctggaattggggatcatcgacgagattgttcccgaaccgccaggcggcgcgcataccgatatgggcgccatagttgcgaccctggggaactatctccgccgccatctgaccgaactgctggcgctggatgtcgcaacgctgttagagcgtcgctatgcgcgctatcgggcgattggaaggtacgaagaagacagtcgccaggctgtcttcgcgtag

SEQ ID NO: 112 - DNA, 972 bp, Ammonifex degensii
atgcctacttacctggacttcgagcggccgctggtagaactggaagagaagatagcggaactcacctctttcagccaggaaaaggggctggacctcagtgaggagatagccaagctccaccagcgggccgaggagctaaaaagacaaatctttgcccaccttactccctggcaaaaggtgcagctggcccggcatcccgaccggcctaccacactcgattatatccgcctgctctgtgaggagttcaccgaactgcacggcgaccggctatacggggatgacccggcaatagtaggaggtatagcctggtttgccgggagaccagtcatggtcctggggcatcagaagggcaaggatacgcgggagaacatgcgccgcaacttcggcatgccccatccggaaggcttccgcaaggcgcttcgcctcatgctgcaggcggagaagttcggccgccccatcattacctttatcgatacccccggcgcctactgcggcatcggggcggaggaacgggggcagagtgtagccattgcccagaatctggcgcgcatgagttccttgcgcgtccccataatagtggtagtcatcggtgaagggggaagcgggggagctttggccctggcggtaggggatcggattttgatgcaggagcacgccatcttctcggtcatctccccggaggggtgcgccagcattctctggaaggacgccagccgggcgaaggaagcggccgaggcgttgaagctcacagcgcaggaccttctggccctgggcctcatcgacgaggtgatacctgagcctttagggggagcccaccgcaaccgcgagggggcggccgaattcctgcgggaagctctcttacggcacctagaggaactggagggtattccgccggacgaactctgccgtcaaagatacgccaaataccgccgcgtaggtcctaccttttaccagggggagagggaagcgtga

SEQ ID NO: 113 - DNA, 885 bp, Sphaerobacter thermophilus
atgcctgagaccctctctgcctgggagcgcgtgctcctggcccgtaacccggcacgcccgcatacgcaggactacgtggcggacctgatctccgggttcgtggaactgcacggcgaccggcgcttcggtgatgatccggccctgctcggcggtatcggcaccttccgcggccgggcggtcgtcgtcgtcggccaccgcaagggggccaacacccaggagaacctggcgcacaacttcggcatgccgcggcctgagggctaccgcaaggcgctgcggctgatgcagcacgccgagaagttcggcatgccgctcatcgccttcgtcgacacgccgggcgccgaaccgggaatcggctcggaggagcgcggccaggccgtcgccatcgccgagaacctgctggcgcttgcctccctgcgcgtcccgacgctggccgtcgtgatcggcgaaggggggagcggcggcgcgctcgccatcagcgtggccgaccgcatcttgatgctggagaacgcgatctatgccgtcgcgtctcccgaggcctgcgcgacgatcctctggaaggacgtgagcaaggctcccgaggcggccgcgaccatgcgcgtcaccgccgccgacctgtacggcttcggcatcgtcgatgaggtgatccccgagccggcccccgcccacgagcagccgacccagaccatccagcgcgtgggcgacgctctggaacgccaccttgccgagctggaggcgctggtgcacgacggagacgccggcatcgacgcgctgctcgcggcccgctaccagaagtaccggaggatcggccgctgggtcgaggagaccgacccgacccgccaccccaacggaccgatcccgtcccacgctgccgaccaagcccgctaa

SEQ ID NO: 114 - DNA, 867 bp, Roseiflexus sp
atgaaaacgccagccttgacgccatgggacagggtgcaactggcgcgtcacccgcgccgtccgcatacgctcgattatgtgcgcgccctgtgcgacgattttatcgaactccacggtgatcgtcgctacggcgacgatccggcgatcattggcggaccggcgcgcttcgccgaccgcacggtcatgatcgtggggcatcagaaagggagcgacgcgcgcgagaatgtgcggcgcaactttggtatggcgcgcccggaagggtatcgcaaagcgctgcgcctctttcgccacgccgaaaagtttggctttccgctgatctgcttcatcgatacgccaggcgccgatccgagcatggaatccgaggagcgcggtcaggggaatgctattgcggaaaacattctggcgctcgccgggttgcgcgtcccgatcgttgcatgtgtgatcggcgagggggggagcggcggcgcgctggcgcttggcgtcgccgatcggctcctgatgctcgaacacgcaatctactccgtggcagcgccagaagcggcagcgtcgattctctggcgcgacgcaggcaaagcgccggaagcggcaagcgcaatgcgcattacggcgcaggaccagtacgacctggggatcgcggatgcgatcattcccgagccagagggcggtgcgcacaccgatgccgccgccgcagcggaggcagtgggggcagcgctgcgtgcagcgctcgatgcactggtcgggctgccgatcgatgtcctgttgcagcgccgctacgccagataccgcgctatcggacgctttcaggaaagccaaccaatgccgaacatgcctgccaccgttccgccgcttcccttcaagggatcgtaa

SEQ ID NO: 115 - DNA, 837 bp, Herpetosiphon aurantiacus
gtgacaacgttaactgagttattgccttgggataaggtgcaaatcgcccgtaatccccaacgcccacgcacccttgattatattcgggtgctgtgtgaagatttttttgaactccgtggtgatcgccatcatggcgatgatcaggcgctggtctgtggtataagcaaaatcgatggccgcagcgtggcgatcatcggccatcaaaagggcagcgacaccaaagagaatgttcggcgcaacttcggctcaccacaccccgagggctatcgcaaggccttacgggtgatggagcatgccaaaaaattcaacatgccgattttgacattaattgatactgctggcgcacacccaagcatggccgccgaagaacgcggccaagccgaagctatcgcccgtaatttattggttatggccgatttgccagtgccaattattgcgaccgtgatcggcgaaggcggctctggcggtgctttggcgattggggtcgccgaccgtttgttgatgcttgaacatgctgtctattcggtggcctcgcctgaagccagtgccgcaattctctggcgcgattcgagcaaagcctcagaagctgccaaagctatgaaaatcaccgcccaagatctccacagctttggcattgctgataccattattcccgagccagagggtggggcgcatatggatgtgccagtaattttacaagcagttgctagcgcactgcgtgagcaaattacgaccctgagtaccctcacaatcgatcaattgcttgaacagcgctatgctaaatatcgggcaattgggcgctttcgccaagcccaaaccacactggttgaaggctaa

SEQ ID NO: 116 - DNA, 864 bp, Roseiflexus castenholzii
atgaccacgccaactctctctccatgggataaagtgcaactggcgcggcatccacgccgtccgcacacgctcgattatgtgcgtgcattgtgcgaagatttcgtcgaattgcacggcgaccggcgctacggcgacgatgaggcgattgtcggcggtctggcgcgctttgccaaccgcaccgtcgtgatcgtcgggcatcagaaaggaagtgatgcgcgggagaatgtgcgccgcaattttggtatggcgcacccggagggataccgcaaggcgctgcgtctctttcgcctcgccgaaaaattcggctttcccctgatctgtttcatcgacacgcccggcgccgatccaagcatggaatcagaagagcgtgggcagggcaatgcgattgcggaaaatattctggcgctcgccgggttgcgggtcccgattgtggcatgtatcattggcgaaggtggcagcggcggcgccctggcgctcggcgtcgccgaccggctgctcatgctcgaacacgctatctattccgttgcggcgcccgaggcagctgcctcgatcctgtggcgcgacgcgagcaaagcgcccgaagcggcgagcgcgatgaaaatcactgcgcaggatcagtacaacctcggaattgttgatgagatcgttcccgagccggacggcggggcgcataccgatgccgcaacaacagcagcggcggcaggaaaagcgttgcgcgccgcactcgacgagttgaccgcgctgccaatcgacgaactgctccggcgccgctacacccgctatcgcgccatcgggcgattccaggaacatcaaccacgtctgctcgacctgcgttccccgccgttgccgttcaagggatcgtga

SEQ ID NO: 117 - DNA, 918 bp, Chloroflexus aggregans
gtgaaagagttcttccgcctcagccgcaaaggatttaccgggcgcgacgatgtagatagcgcccaaatcccagacgatttgtgggtgaagtgtagcgcatgtcgtgagctgatctacaagaagcagcttaacgacaacctgaaggtttgtccgaaatgtgggcaccatatgcggatgagtgcccacgagtggattggtctgctcgatgtcggttcgttccgggaaatggattccaacttactaccaaccgacccgctcggtttcgtcgctgccgatgagagctacgccaccaagttagctaaaacgcagcagcgcaccggtatgaccgatgcagtgatctccggtgtcggtgctatcagtggtatccggctctgcatcgctgtggccgatttctcgtttatgggcgcctcgatgggtagcgtctacggtgagaaaatggcccgcgctgccgaacgtgcagccgaactaggcataccgctgttaacgattaatacctcgggtggcgcccgccagcaggaaggtgtgatcggcttgatgcaaatggcaaagattacgatggcacttactcgccttgccgaagccggtcagcctcatattgctctgctggtcgatccatgctacggtggtgtgactgcctcgtatccctcagtagccgatattattatcgccgaaccgggtgccaatatcggttttgccggcaagcggttgatcgagcagattatgcgacaaaagctgccggccggttttcagaccgccgagtttatgctggaacacggtatgatcgatatggtggtgccacggagtgagatgcgagaaacacttgctcgcattctaaagcactatcagcagcgacaagcaccagcagcgaaagccgatcttgcagcacggcgtgcaacgttgccgcaaccgattatgtag

SEQ ID NO: 118 - DNA, 912 bp, Oscillochloris trichoides
gtgaaagagtttttccggcgacgaaagaccagcttcaccacgggcgaacagcctgagggtaatccagttcccgatgacatgtgggtgaagtgcgcagcatgccgcgatctcgtctacaagaaggaactgattgacaacctgaaggtgtgtcccaaatgtagccaccacatgcgcctgagtgcgcgtgagtggctcgatctcctcgacaccgacaccttcagcgagaccgacacccatctacggcccaccgacccactagggtttgtgtcgcttgacgagagctacaccgataagctggttaaatcccaggagcgcaccggcatgcccgatgcggtgattgcggggctaggcatgatcggcggccagcgcttggcattggcggtgggtgactttgccttcatgggggcttccatgggcagcgtctatggcgagaagatgtcacgcgcagcggagcgcgccgccgacctgggcattcccctgctcaccatcaacacctcaggtggcgcacgccagcaagaaggtgtcatcgccctgatgcagatggccaaagtgaccatggcgctcacccgcttggctgaggccggccagccccatattgcgctgctggttgacccctgctatggtggggtgacggcctcctacccctcagtggccgatgtgatcatcgccgagccgggggccaacatcggctttgcaggcaagcgcctgatcgagcagattatgcgccagaagttgccgtccggcttccagaccgccgagttcatgcttgaacatggcatggtagatatggtagtcgaacgcaaccaactgcgggccaccctagcacgcctcttgcggctctatgccgggcgacgcgagccagttgcaccattcatggtatcatcggaaatctacgtcaacggacgcgcataa

SEQ ID NO: 119 - DNA, 987 bp, Roseiflexus castenholzii
atgctgctatccgtttctcatgtggtcaggcgctccgggggttatgcagggcgaccgcacagtctgtggcaaggagaggactcgatgaaagagttgattcaacgatcgcggaagagcttcaccgtcgtgcatccgatcgagtcggacgtgccggacaacgtctgggtcaagtgcccgtcgtgccgggaattgatctaccacaagcaactggctgagcgcatgaaggtgtgtcgctgcggctaccacatgcgcctgacagcgcgtgagtggctggcgctgctcgatgaaggttcgttcgtcgaatacgacgcgcacctgcgccccaccgacccgctcggctttgtgtcgcccaaagaagcatatgccgataaactgcgcgaaacgcagcgtcgcaccggtcttgctgatgtcgtggtgagcggcgttggcagcattgaaggataccgactgtcgattgcagtgtgcgatttcaacttcatcggcggctcgatgggcagcgtcttcggcgagaagatggcgcgcgctgcggagcgcgcggcgacgctcggcattccgctgctcacgatcaacaccagtggcggcgcgcgcatgcaggaaggcgtgatcgcgctgatgcaactggcgaaagtcaatatggcgctcacccgtctggcggcggcgcgtcaaccgcatatcgccgtacttgtcgatccatgctatggcggcgtcactgcctcctacgcctcagtcgccgacattattattgccgagccgggcgccaacatcggctttgccggtcgccgtgtgattgaacagacgattcgccagaaactgccggcggattttcagactgccgaatttatgctccagcacggcatggtcgatatggtcacaccgcgcagcgagttgcacggtgtgctggcaaaactgctgcgcctctacgccgccgaagggcgcagtggagcgtattcctccgaagcggtcgcaccgacgctggcatccatctga

SEQ ID NO: 120 - DNA, 903 bp, Roseiflexus sp
atgaaagagttgatccaacgatcgcgcaagagtttcaccgtcgtacaatccgtcgaggcggatgttccggacaatgtctggatcaagtgtccatcgtgccgcgaactgatctaccacaaacaactggcggaacgcatgaaggtttgccgctgcggctaccatatgcgtctgaaggcgcgcgagtggctggcgctgctggatgaagactcgttcgtcgagcacgacgcccacctgcgtccagccgatccgctgggttttgtgtcgcccaaagagacgtatgccgacaagttgcgcgaggcgcagcgccgcaccggtcttgccgatgtagtggtcagcggcgttgggagtatcgaagggcgccgtctggcagttgcagtatgcgactttgaattcatcggcggctcaatgggcagcgtgttcggcgagaaaatggcgcgtgcagcggaacgcgctgcggcgctcggcattccgctgctcacgatcaataccagtggcggcgcacggatgcaggaaggggttattgcgctgatgcaactggcaaaagtcaacatggcgctgacccgccttgccgctgcccgtcaaccgcacatcgccgtgctggtcgatccctgctatggcggcgtcaccgcttcctacgcttcggtcgccgacatcattattgccgagccgggcgccagcatcgggtttgccggtcgtcgcgtcatcgagcagacgattcgccagaaacttccggcagatttccagactgccgagtttatgcttcagcacggcatggttgatatggtcgttccgcgcagtgagttgcacagcaccctggcgaaactgctgcgcctctacgcggctgaaggtcgtgccacagcgcacaagtccgaaccgatcgtaacagcgttggcttctctctga

SEQ ID NO: 121 - DNA, 903 bp, Roseiflexus sp
atgaaagacctgtttcgtcgcgcgccaaagcgttttacggcagcgcgcatcgagaatcaggcgatccccgacaatatgtgggtcaaatgcccgtcctgcggcgacctgatctatacccggcagttcagtgacaatctgaaagtctgcaaatgcggctatcacatgcgtctgaccgcgcgtgagtggcttggattgctcgatgacgggtcgttcgtcgagttcgatgctgccctggcgtcggtcgatgcgctgggattcgtgtcgccacgtcatgtctacgagcagaaattgatcgaaagtcggcagcagactggcctgaacgatgcgctgatcaccagcagcggagcgatcaacgggatgctgctatgcctggcagtcaccgaattcgagtttatcggtggctcgatgggaagcgcctatggcgagcgcctggcgcgcgtgatcgagcgcgccgccgatgcgcgcctcccgcttttgacgatcaacgccagtggcggtgcacggcaggaagaaggcacgctggcgctcctccagatggcgaaggtcaacatggcgctaacgcgccttgccgccgccggtcaaccccatatcgctctgctggttgatccatgctatggcggcgtgctggcgtcgtacacctcggtcgccgatgtgatcatcgccgaacctggcgcgcgagtcggctttgccgggcgacgggtcatcgagcagacgatccgccagaaactgccggtccactttcagaccgccgaattcctgctggatcacggcatgatcgatatggtgacgccgcgtagtgaactgcggggcgtcctgtcaaccctcctgcgattgtatcgtgatgcgtttgcacacacgtcagcagcgcagcatgttccggcgctgacgcacgtccagggatga

SEQ ID NO: 122 - DNA, 897 bp, Roseiflexus castenholzii
atgaaagacttgtttcggcgcgcgccgaagcgtttcacggcagcgcgcatcgagaatcaggcgatccccgataacatgtgggtcaagtgcccttcgtgcggcgatctgatctacaccagacagttcagcgacaatctgaaggtctgtaagtgcggctatcacatgcggctggcagcgcgtgaatggttggggctgctcgacgatggttcgtttgtcgagttcgacgccgcgctggcgccggtggatgcgctggagttcatatcgccgcgtcacgtctacgcgcacaagttgagcgaaagccaggagcagaccgggctgaatgatgcgcttatcaccggcagcggcgcgattgagggcatgccattatgcgtggcagtcaccgaattcgagttcattggcggttcgatgggcggtgcatttggcgagcgcctggcgcgtatcatcgagcaggctgccgacgcacgcgttccgctgttgacgatcaacgccagcggcggtgcgcgccaggaagaaggaaccctggcgctgctccagatggcgaaagtcaatatggcgctgacccgccttgcagcggtcggtcaaccacacattgccgtgctggtcgatccgtgctacggcggtgtgctggcgtcgtacacatctgtggcagacattatcatcgctgaacccggcgcgcggatcgggttcgccgggcgtcgggtcattgaacaaaccattcggcagaaactgcccgcccattttcagaccgccgaatttctgttgagtcacggtatgatcgatatggtgacgccgcgcggcgaactgcggagcgtcctggcaacgttgctccgcttgttccacaacgcgcccgaacgcgccgcagacgttcagaatgccccggcgctggcgcgcgcctga

SEQ ID NO: 123 - DNA, 897 bp, Herpetosiphon aurantiacus
ttgaaagatttttttcggcgtgcgccgttacccttcacgtcttcgcggcgtgagcaacaaataccagataatgtttgggccaaatgcgccaattgtggcgaattgacctatcaaaaacaattcaatgatgccttaaaagtctgtccaaaatgcagctatcactcgcggattagctcacgcgaatggattgaagttttggctgatgctgattcattcgtcgaatatgatgctgatctacaagggatcgacattttgggctttgttagccccaaagataattacgaagcgaaattagctgccaccagcgaacgcactggcactaacgatgttgtgatgagcggtagcgccagcatcgaaggcttgccatttgaaatcgcagcctgtaattttgaatttatgggcggttcgatgggcagcgtctttggcgaaaaagtggcacgagcagtcgaacgagcagccgatcgtggcgtaccagtgctgacgatcaacgcttcgggtggcgctcgtatgcacgaaggcatttttgccctgatgcaaatggccaaagtttcggttgcgctcacccgtttggctcgggtgcgccagccccatatctcgctcttagttgacccttgctatggcggggtttcagcctcgtatgcctcggttgccgatattattttggccgaaccaggtgcgaatattggctttgctgggcgacgggtgattgagcaaacgattcgccaaaaattgccaccaaacttccaaaccgcagaatttttccttgaacatggcatgatcgatgcagttgtgccacgctcggacatgcgggcaaccatcgggcgtttgttgcgcctgtatcaacgccccacatcatcggctgatcatcgcgaacatgtcgtagcgggccaccaataa

SEQ ID NO: 124 - DNA, 921 bp, Sphaerobacter thermophilus
atgcgggaactgttccgccgacaaccgcgcttcacgtctgaaccacagagcgaggacagcccggtcgtacccgatgacctctgggtcaagtgcccacgctgccgcgagttgacctactcgcgcgagttcgagcgcgaactgcgggtttgcccacgctgcaaccaccacttccgcctcaccgccgcgcagcggatcgcgatgctgaccgacccgggcagcttcgtggagtgggacgccgggctcgaagcggccgacccgctcgggttcgccgccggcggcgagtcctaccccgacaaagtagcgacggccaagcgtaagagcggtacgcgcgaggcgctggtcaccgggtccgcccggctggacggtcgcccgctcgcgctggcggtcgcggaattcggcttcatgggcgccagcatgggctccgtcttcggggagaagctggtgcgggcgatcgagcgggccatcgagcaggagctgccgctggtcacggtctcgtcgtccggcggtgcgcgcatgcaggagagtcccttctcactgatgcagatggcgaagaccacagcggcgctggcgcggctgggcgaagcccggctgccccacatcgcggtgctggtggacccgtgctacggcggggtcacggccagctacacgacggtggccgacgtcatcatcgcggagccgggcgccatgatcggcttcgccggccctcgtgtcatcgagcagatcacccggcagaagctccccgagggcttccagaccgccgagttcctgctggagcacggcatgatcgatctgatcgccccgcggcgcagcctgcgcgcgaagatcgcgaccttgctcgaccactacgcattggcccgccagcggccagcccgccggcctgccgtcgcggccgcctcggcgcaggaggcgcccgatgcctga

SEQ ID NO: 125 - DNA, 1548 bp, Nitrosopumilus maritimus
atgcattctgaaaaactcgaaaattataacaacaaacacaaaacttctcaacaaggcggtggtcaggataggatcaaagcccagcatgataaaggcaaattaactgcccgagaaagaatcgatcttctattagatgagggtagttttaccgaaatcgacccaatggtaactcatcattatcatgaatatgatatgcagaaaaagaagttctttactgatggtgtagttggtggttatggaaatgtcaacggtagacaaatcttcgtatttgcttatgatttcactgttcttggtggaactctaagtcaaatgggtgccaaaaaaattactaaattaatggatcatgcagttagaactggatgtccagttatcggaatcatggattctggtggtgcaagaattcaagaaggaataatgagtcttgatggatttgcagatattttttatcataatcaattagcttcaggagttgttcctcaaattacagcaagtattggtccttctgcaggtggctcagtgtattcaccagctatgacagactttgtcgtaatggtggaaaaggcaggatcaatgtttgttactggtcctgatgttgtaaagacagttttgggtgaagaaatttcaatggatgatcttggtggagctatgacacatggttcaaaaagtggagttgcacattttgttgcacaaaacgaatacgaatgtatggattacatcaaaaaattaatctcttacataccacaaaataattctgaagaaccaccaaaaataaaaaccgatgatgatccaaacagattagataataatctcattaatgttatcccagaaaatccattacaaccttatgatatgaaagaaattatcaattctattgtagataatcatgagttctttgaagttcatgaattatttgcaccaaatattgtcgtaggttatgctagaatggatggtcaagtagttggaataattgcaaacaatccaatgcatttagcaggagcacttgacattgattcatcaaataaatctgcacgtttcattagattctgtgatgcatttaacattcccattatcactttagtagatacaccaggttacatgccaggttctaatcaagaacacaacggtatcattagacatggtagtaaattgctctatgcatactgtgaagcaaccgttccaagaattacacttgtaattggaaaggcatatggtggcgcatacattgcaatgggaagtaagaaccttcgaactgacattaattatgcatggccaactgcacgttgtgctgtactaggtggtgaagctgctgtaaaaatcatgaacagaaaagatttggcagacgcagataatcctgaagaacttaagaagaaattgattgatgagtttacagaaaaattcgaaaacccatacgttgcagcatcacacggaacagttgataatgtaattgatcccgcagaaacaagacctatgttgattaaagccctcaaaatgcttgcaaacaaaagagaaaaacaacttcctagaaaacatggaaatatcaatttgtga

SEQ ID NO: 126 - DNA, 1548 bp, Nitrosoarchaeum limnia
atgcattttgagaaaattgagggatattctaaaagaaataaaacatcatcactgggtggaggtcaagataaaattaaagatcaacatgataagggtaagttaactgctcgtgagagaattgatcttttattggattctggtacttttacagaaattgatcctcttgtaactcatcattactatgagtatgatatgcagaagaaaaaattcttcactgatggtgttgttggtggttatggaactgtaaacggtaggcaaatctttgtctttgcttatgactttactgttcttggtggaaccttaagtcaaatgggtgctaaaaaaattacaaaattaatggatcatgcaatcaagacaggctgtcctataattggaatcatggactcgggtggcgcaagaattcaagaaggaatcatgagtcttgatggatttgcagatattttttatcataatcaactggcttctggagtaatacctcaaattactgcaagtataggtccatcagctggtggttctgtttattctccagcaatgactgattttgttattatggttgataaagtaggaactatgtttgttacaggacctgatgtagtaaagactgttctaggtgaagaaatttcatttgatgatttaggtggagcaatgacacatggaataaaaagtggcgttgcacattttgttgcaaaaaacgaatacgaatgtatggattatattaaaaaattaatttcttttcttccacaaaataatactgaagaaccaccaaaaataaaaactgatgatgatccaaacagacttgatcataatctgatcaatattattcctgataatccattacaaccatatgatatgaaagaaattatttcctccgttgttgacaatcatgaattctttgaaatccatgaattatttgccccaaatgttgttgttggctttggaagattaaatggcaaagttgtcggtatcgttgctaatcaaccgcttcatcttgcaggtgcattagatattgattcatcaaacaaggcagctagattcattagattttgtgattcatttaacattgcaatcattactctagtagatactccaggttacatgccaggttctaatcaagaacacaacggtatcattcgacatggtagtaaacttctatatgcttattgtgaggcaaccgttcctagaattacacttgtaattggaaaagcatatggtggtgcttacattgctatgggaagtaaaaatctgagaactgatgtgaattatgcatggcccactgctcgatgtgctgtacttggtgctgaagctgctgttaaaattatgtatagaaaggaactatcttcctcaaaagatgcagaatctcttaaaaaacaactcatcagcgaatttgcagaaaaatttgaaaatccatacgttgcagcatctcatggaactgttgataatgtgattgatcctgcagaaacaagacccatgttaataaaagcactagaaatgcttgccaataaaagagaaaaacaacttccaagaaaacatggaaatatcaatctgtga

SEQ ID NO: 127 - DNA, 1548 bp, group I marine Crenarchaea
atgcattctgacaaaattaatgaatttttaaagaaaagaaagacggctgaccaagcaggtggtcaggatcgtattgccaaacagcatgagaaaggaaaactcacggcaagggaacgaattaatctattgcttgacgagggaagttttgtcgaacttgatgcactagctacacatcattactatcagtatgatatgcagaaaaagaaattctttggtgatggtgtagttggtggatatggaatgattaacggacgacaggtatacgtttttgcttacgattttacaattttgggaggcactctaagcaaaatgggtgcaagaaaaattaccaaattaatggatcatgctatccgcaacggatgtccaattattggaataatggattcaggcggcgctagaattcaggaaggaatacaaagtcttgacgggtttggtgacattttttatcacaaccaattggcatcaggcgttgttccacaaattactgctagtattggaccgtctgctggtggtgcggtatattctccagcaatgactgattttgttataatggtggataaattaggaaccatgtttgttacgggacctgacgttgtaaagacggttcttggtgaggaggtttcgtttgaggaattgggtggagcaatgacacatggtacaaaaagtggtgtggctcactttgttgtaaaaaatgaatacgaatgtatggattatatcaagacgttactatcatacatcccacaaaacaatactgaagagccttcaactgtacaaaatgatgacgatccaaacaggctggatcataatctaattaaccttcttcctgatgactcgttaaaaccctacgatatgaaagaaattattcattcaatagttgatgatcacaacttttttgaggttcatgaattatttgcacaaaacattattgttggatttgctagaatgcatggaaaaacagttggaatagttgcaagtcaaccattatttcttgctggcgcacttgatattgattcctcaaacaaagctgcgcgctttattagattttgtgattgttataatattccaattgttaccttagttgatactcctggctacatgcctggaacagaccaggaacataacggaattattcgccatggcagcaaacttttgtacgcttattccgaggcaacaattccaaagattactattgtaattggaaaagcatatggtggcgcttacattgcaatgggtagcaagaatctaagaacagacattaactatgcttggccaaccgcacgaattgcagttctgggttctgaaggagctataaatattatgaacaggaaagaacttgccagttcaaaggatcctgaatcattgaaaaaacaattgatagatgagtttactgaaaaatttgccaacccatatgttgccgcatctaatggaacaattgatacagttattgatcctgctgaaacaagacctatgattattaaagctcttgaaatgctttccaacaaacgagacagtcaactgcctcgaaagcatggcaatatgaatctgtga

SEQ ID NO: 128 - DNA, 1548 bp, group I marine Crenarchaea
atgcattctgacaaaattaatgaatttttaaaaaaaagaaagacggctgaccaagcaggtggtcaggatcgtattgccaaacagcatgagaaaggaaaactcacagcaagggaacgaattaatctgttgcttgacgagggaagttttgtcgaaattgatgcactagctacacatcattactatcaatatgatatgcagaaaaagaaattctttggtgacggtgtagttggtggatatggaaatatcaatggacgacaagtctacgtttttgcttatgattttacgattttgggaggcactctaagcaaaatgggtgcaaaaaaaattaccaagttaatggatcatgctatccgcaacggatgtccaattattggaataatggactcgggcggcgcaagaatccaggaaggaatacaaagtcttgacgggtttggtgacattttttatcacaatcaattggcatcaggtgttgttccacaaattactgccagcattggaccgtctgctggtggtgcggtatattctccagcaatgactgattttgttataatggtagataaattaggaaccatgtttgttacgggacctgacgttgtaaagactgttcttggtgaggatgtttcgtttgacgaattgggtggagcaatgacacatgctaccaaaagcggtgtggctcacttcgttgtaaaaaatgaatacgaatgtatggattatatcaagacgttactatcatacatcccacaaaacaatactgaagagccctcaactgtacaaaatgatgacgatccaaacaggctggatcataatctaatcaacattcttcctgaagactcgctaaaaccctatgacatgaaagaaattattcattcaatggttgataatcacaactttttcgaggtccatgaattatttgcacaaagcattattgttggatttgccagaatgcatggaaaaacagttggaatagttgcaagtcaaccattatttcttgccggtgcacttgatattgattcctcaaacaaagctgcgcgcttcattagattttgtgattgttataatattccaattgttaccttagttgatactcctggttacatgcctggaacagaccaggaacataacggaattattcgtcatggcagcaaacttttgtacgcttattccgaggcaacaattccaaagattactattgtaattggaaaagcatatggtggcgcttacattgcaatgggtagcaagaatctaagaacagacattaactatgcttggccaactgcacgaattgctgttctaggttctgaaggagctgtgaatattatgaacaggaaagaacttgccagttcaaaggatcctgaatcattgaaaaaacaattgatagatgagtttactgaaaaatttgccaatccatatgttgccgcatctaatggaacaattgatacagttattgatcctgctgaaacaagacctatgattattaaagctcttgaaatgctttccaacaagcgagacagtcaactgcctcgaaagcatggcaatatgaatctgtga

SEQ ID NO: 129 - protein, 201 aa, Artificial Sequence
  amino acid sequence of the Biotin Carboxyl Carrier Protein from Cenarchaeum symbiosum with N-terminal linked chloroplast protein from Ricinus communis stearoyl-ACP desaturase.
MALKLNPFLSQTQKLPSFALPPMASTRSPKFYMKYEIEDAGSFEGRMAANPGNGEYTLEINGKEVRLKVISMGPRGMEFLLDQKYHSARYLERSTSGIDMIIDGTPVRAGMHADLDKIVYKNSGGGGGGGPGIALRSQIPGKVVSLEVSEGDEIKKGDPVAVLESMKMQVAVKAHKDGTVKSVSIKEGGSVAKNDVIAEIE

SEQ ID NO: 130 - DNA, 606 bp, Artificial Sequence
  nucleotide sequence of the Biotin Carboxyl Carrier Protein from Cenarchaeum symbiosum with N-terminal linked chloroplast protein from Ricinus communis stearoyl-ACP desaturase (codon optimized for expression in Arabidopsis thaliana).
atggccctcaagttaaatccattcctttcacagactcaaaaattaccttctttcgcactccctcctatggcatcaaccagatcacctaagttttatatgaagtacgaaatagaggatgctggttcttttgaaggaagaatggctgcaaatcctggtaacggagagtatactcttgaaattaacggaaaggaggttagactcaaagtgatatctatgggacctagaggtatggaatttcttttggatcaaaagtaccattcagctagatatcttgaaaggtctacttcaggaattgatatgattatcgatggtacacctgttagagctggaatgcacgcagatttggataagatcgtgtacaaaaactcaggaggtggaggtggaggtggacctggtatagctcttaggagtcaaattccaggaaaggttgtgagtttggaagtttctgagggagatgaaatcaagaaaggagatccagttgcagtgcttgagagtatgaagatgcaggttgctgtgaaggcacataaagatggtacagttaaatcagttagtattaaggagggaggtagtgtggcaaagaacgatgtgatagcagagattgaatga

SEQ ID NO: 131 - protein, 508 aa, Artificial Sequence
  amino acid sequence of the Biotin Carboxylase from Cenarchaeum symbiosum with N-terminal linked chloroplast protein from Ricinus communis stearoyl-ACP desaturase.
MALKLNPFLSQTQKLPSFALPPMASTRSPKFYMIRTCRALGLGSVAVYSDEDYNALHVKKASESYHIGGAAPAESYLNQQRIIEAALSSGADAIHPGYGFLSENGEFAALCEKNRINFIGPSAKSMNLCGDKMECKAAMLKADVPTVPGSPGLVGSADEAAGIASKIGYPVLLKSVFGGGGRGIRLAEDEGGLRGGYDSATAESIAAVGKSAILVEKFLKRTRHIEYQMARDKHGNAVHIFERECSIQRRNQKLIEQTPSPVMDEDTRKRIGDLVVKAAEAVDYTNLGTAEFLRADSGEFYFIEINARLQVEHPITELVSGLDLVKLQIDIANGEPLPFKQNDLRMNGYAIECRINAEDTFLDFAPSVGPVPDVKLPSGPGVRCDTYLYPGCTVSPFYDSLMAKLCTWGATFEESRLRMLGALGDFYVEGVETSIPLYKTIMASDEYKNGELSTDFLSRYNIIDRLDKDIKKERAANGEAAAAAAIMHSEFLSSRAGGNSGTAWKGGA

SEQ ID NO: 132 - DNA, 1527 bp, Artificial Sequence
  nucleotide sequence of the Biotin Carboxylase from Cenarchaeum symbiosum with N-terminal linked chloroplast protein from Ricinus communis stearoyl-ACP desaturase (codon optimized for expression in Arabidopsis thaliana).
atggccctcaaactcaaccctttcttatctcaaacccaaaaactcccttcattcgctcttcctcctatggcatctaccaggtctcctaaattctatatgattagaacctgtagggctctcggattaggttctgttgcagtgtattcagatgaagattacaatgctcttcatgttaagaaagcaagtgaatcttatcacattggaggtgctgcaccagctgaatcttacctcaaccaacagagaattatcgaggctgcattatcttcaggtgctgatgcaatccatcctggatacggatttctttcagaaaacggagagttcgctgcactctgcgaaaagaatagaattaatttcattggtccatcagctaaaagtatgaacttgtgtggagataagatggagtgcaaagctgcaatgctcaaggctgatgttcctacagtgccaggaagtcctggtttggttggatctgctgatgaagctgctggaatcgcatcaaagataggatatcctgtgcttttgaaaagtgttttcggaggtggaggtagaggtattaggttggctgaagatgagggaggtctcagaggaggttacgatagtgctacagcagagtctattgctgctgttggaaaatctgctatcctcgttgaaaagttccttaagagaaccaggcatatcgagtatcaaatggctagagataagcatggtaatgcagttcacatcttcgaaagggagtgttcaatacaaagaaggaaccagaaactcatagaacagacccctagtccagtgatggatgaggatactagaaagaggattggagatttggttgtgaaagctgctgaagctgttgattatactaatcttggtacagctgagtttcttagagcagattctggagaattttacttcatcgagataaacgctaggttacaagttgaacacccaatcactgagcttgtgtcaggtcttgatttggttaagttgcaaatagatatcgctaacggagaacctttaccattcaaacagaatgatcttaggatgaacggatatgctattgaatgtaggatcaacgcagaggatacatttcttgatttcgctcctagtgttggacctgtgccagatgttaaacttccatctggacctggtgtgagatgtgatacatatttgtacccaggttgcaccgtttcacctttttatgatagtttgatggctaaactctgcacctggggagcaacttttgaagagtcaagacttaggatgttaggtgctcttggagatttctacgtggaaggtgttgagacttctattccactttataagacaattatggcttcagatgaatacaaaaacggagagttgtctactgatttcctctcaagatacaacataattgataggttggataaggatatcaagaaagaaagagctgcaaatggtgaagctgctgctgctgctgctattatgcactctgagtttctctcaagtagggcaggaggtaatagtggaacagcatggaagggtggagcataa

SEQ ID NO: 133 - protein, 547 aa, Artificial Sequence
  amino acid sequence of the Carboxyltransferase from Cenarchaeum symbiosum with N-terminal linked chloroplast protein from Ricinus communis stearoyl-ACP desaturase.
MALKLNPFLSQTQKLPSFALPPMASTRSPKFYMHSEKLDKRSANNRSALMGGGEARIEAQHGKGKLTARERIAIMLDEGSFTEVDSLATHHYHEFDMQKKKFFGDGVVGGYGRIDGRKVFVFAYDFTVMGGTLSQMGAKKITKLMDHAVRTGCPVIGVMDSGGARIQEGIMSLDGFADIFYHNQLASGVVPQITASIGPSAGGSVYSPAMTDFVIMVEKSATMFVTGPDVVQTVLGESISFEDLGGAMTHGSKSGVAHFVAKNEYDCMDYIRKLLSFIPQNNREEPPVVKTADDPDRLDHGLIGMIPENPLQTYDMKNVIHSIVDDRTFLEVHENFATNIIVGFGRFNGRAAGIVANQPASLAGALDIDASSKAARFIRFCDAFNIPVITLVDTPGYMPGSDQEHGGIIRHGSKLLFAYCEATIPKITLVIGKAYGGAYIAMASKNLGTDINYAWPTARCAVLGAEAAVKIMNRKDLAAASDPEGLKKELIGNFAEKFDNPYVAASHGTVDAVIDPAETRPMLIKALEMLSSKREGRISRKHGNINL

SEQ ID NO: 134 - protein, 1644 aa, Artificial Sequence
  nucleotide sequence of the Carboxyltransferase from Cenarchaeum symbiosum with N-terminal linked chloroplast protein from Ricinus communis stearoyl-ACP desaturase (codon optimized for expression in Arabidopsis thaliana).
ATGGCCCTCAAACTCAACCCTTTCCTCTCTCAAACCCAGAAACTCCCTTCATTCGCTCTCCCTCCTATGGCTTCAACCAGGTCACCAAAATTCTACATGCATTCTGAAAAACTTGATAAGAGATCAGCAAATAACAGGAGTGCTTTGATGGGAGGTGGAGAGGCAAGAATCGAGGCTCAACACGGTAAAGGAAAGCTCACTGCAAGAGAAAGGATCGCTATAATGTTAGATGAAGGATCTTTTACCGAGGTTGATTCACTTGCTACTCATCACTACCATGAGTTCGATATGCAGAAGAAAAAGTTTTTCGGAGATGGAGTTGTGGGTGGATATGGTAGAATTGATGGAAGGAAGGTTTTTGTGTTCGCTTACGATTTCACAGTTATGGGTGGAACCCTCTCTCAAATGGGTGCAAAAAAGATAACTAAACTTATGGATCATGCTGTTAGAACAGGTTGTCCTGTTATCGGAGTGATGGATAGTGGTGGAGCTAGGATACAGGAAGGTATTATGTCTCTTGATGGATTCGCAGATATCTTCTATCACAACCAATTGGCTAGTGGTGTTGTGCCTCAGATCACTGCAAGTATAGGACCATCTGCTGGTGGATCTGTTTACTCACCTGCAATGACAGATTTTGTTATCATGGTGGAGAAATCAGCTACTATGTTCGTTACAGGTCCAGATGTTGTGCAAACTGTGCTTGGAGAAAGTATTTCTTTTGAGGATTTGGGTGGAGCTATGACACATGGTTCAAAAAGTGGAGTTGCACACTTCGTGGCTAAGAACGAATACGATTGCATGGATTACATAAGAAAACTTTTGTCTTTTATCCCTCAAAATAACAGGGAAGAGCCTCCAGTTGTGAAGACTGCTGATGATCCAGATAGACTTGATCATGGTTTGATTGGAATGATCCCTGAGAATCCACTCCAGACTTATGATATGAAGAACGTTATACATTCTATAGTGGATGATAGAACCTTTTTGGAAGTTCACGAGAATTTCGCTACTAACATTATCGTGGGTTTTGGAAGATTCAACGGAAGAGCTGCTGGAATTGTTGCAAACCAACCTGCTTCACTCGCAGGAGCTTTAGATATCGATGCTTCTTCAAAGGCTGCAAGATTCATTAGGTTCTGTGATGCTTTCAATATCCCAGTTATAACCTTGGTGGATACTCCTGGTTATATGCCAGGATCAGATCAGGAACATGGTGGAATTATTAGACACGGTAGTAAACTCCTCTTTGCATACTGCGAGGCTACTATACCTAAGATCACACTTGTTATAGGAAAGGCTTATGGAGGAGCTTACATTGCAATGGCTTCTAAGAATTTGGGAACAGATATTAACTATGCATGGCCAACCGCAAGATGTGCTGTTCTCGGTGCTGAAGCTGCTGTTAAGATTATGAATAGGAAGGATCTTGCTGCTGCTTCTGATCCTGAAGGTCTTAAAAAGGAGTTGATTGGAAATTTTGCTGAGAAATTCGATAACCCATACGTTGCAGCTTCACATGGAACAGTTGATGCAGTGATTGATCCTGCTGAAACCAGACCAATGCTCATCAAGGCTTTAGAGATGCTCTCATCAAAAAGGGAAGGAAGAATAAGTAGGAAACACGGTAACATCAACCTCTGA